(19) United States
(12) Patent Application Publication
(10) Pub. No.: US 2012/0207753 A1
(43) Pub. Date: Aug. 16, 2012

(54) METHODS OF USING CD44 FUSION PROTEINS TO TREAT CANCER

(75) Inventors: Qin Yu, New York, NY (US); Ivan Stamenkovic, Lausanne (CH)

(73) Assignees: CENTRE HOSPITALIER UNIVERSITAIRE VAUDOIS, Lausanne (CH); MOUNT SINAI SCHOOL OF MEDICINE OF NEW YORK, New York, NY (US)

(21) Appl. No.: 13/391,144

(22) PCT Filed: Aug. 16, 2010
(86) PCT No.: PCT/US10/45635

§ 371 Date: May 7, 2012

Related U.S. Application Data
(60) Provisional application No. 61/274,813, filed on Aug. 21, 2009.

(51) Int. Cl. A61K 39/395; A61M 37/00; A61P 35/00

(52) U.S. Cl. 424/134.1; 600/1

(57) ABSTRACT

Pharmaceutical compositions and methods for treating cancer using CD44 antagonists are disclosed. In certain aspects, these pharmaceutical compositions and methods include treating a mammal having a cancer, such as glioma, colon cancer, breast cancer, prostate cancer, ovarian cancer, lung cancer, renal cell carcinoma, gastric cancer, esophageal cancer, head cancer, neck cancer, pancreatic cancer, or melanoma, with a CD44 fusion protein. These CD44 fusion proteins include CD44-Fc fusions and can be used to detect hyaluronan.

### RELATED APPLICATIONS

This application claims priority to U.S. provisional application Ser. No. 61/274,813, filed Aug. 21, 2009, which is herein incorporated by reference in its entirety.

### GOVERNMENT FUNDING

The United States Government has certain rights to this invention by virtue of funding received from the Department of Defense, Army Medical Research, Grant No. W81XWH-06-1-0246 and National Institute of Health, National Cancer Institute Research, Grant No. R01CA135158-01A1.

### FIELD OF THE INVENTION

The present invention is related to pharmaceutical compositions and methods for the treatment of cancers with CD44 fusion proteins and the derivatives of these fusion proteins. In certain aspects, these pharmaceutical compositions and methods include the use of CD44 fusion proteins as single agents and in combinations with other anti-cancer therapeutics to treat cancers, including glioma, and to prevent recurrence of cancers, including that of glioma, after a variety of therapeutic interventions including surgical removal of cancers. In other aspects, these pharmaceutical compositions and methods include the use of CD44 fusion proteins along with, prior to, or after other anti-cancer therapies to treat glioma and other cancer types. CD44 fusion proteins can be used after other therapeutic interventions as a maintenance therapy to block expansion of cancer stem cells and to delay or stop cancer recurrence and metastasis. In another aspect, the combination of pharmaceutical compositions or methods administered along with other anti-cancer therapies provides a synergistic effect on the treatment of glioma and other cancer types. In other aspects, these pharmaceutical compositions and methods include the use of CD44 fusion proteins to detect CD44 ligands, including HA, for early cancer diagnosis and prognosis, and for assessment of patient responses to anti-cancer treatments.

### BACKGROUND OF THE INVENTION

Conventional anti-cancer therapy is primarily directed at tumor cell properties that distinguish them from normal cells, including a generally higher proliferation rate and distinct metabolic requirements. Although beneficial in a selected group of malignancies, conventional chemotherapy has a limited effect on the majority of solid tumors while imposing serious toxicity. More recent targeted therapeutic strategies are designed to target specific hyperactivated oncogenes and kinases in cancer cells. They are generally less toxic than chemotherapy but their efficacies are limited by tumor cell heterogeneity, ability to switch their dependence from one aberrant signaling pathway to an alternative one, and emerging of resistant clones that have acquired new mutations. It is thus becoming increasingly apparent that merely targeting cancer cells is unlikely to cure most solid malignancies (Araujo et al., 2007; Zhang et al., 2009). Accumulating data from numerous recent observations indicate the host microenvironment provides an essential contribution to cancer progression, helps maintain cancer stem cell niches, and modulates the response of cancer cells to treatment, implying that elements within the tumor microenvironment may constitute important targets for anti-cancer therapy (Gilbertson and Rich, 2007; Hideshima et al., 2007; Hoelzinger et al., 2007; Mantovani et al., 2008; Mishra et al., 2009; Podar et al., 2009).

The tumor microenvironment consists of the infiltrating host cells, including endothelial cells, pericytes, leukocytes, and fibroblasts, as well as the components of the extracellular matrix (ECM). Key interactions and cross-talk between tumor cells and their microenvironment are mediated by their surface receptors including cell-cell adhesion and ECM receptors that are potentially attractive therapeutic targets. It has been elegantly shown, for example, that adhesion of multiple myeloma (MM) cells to the ECM confers cell adhesion-mediated drug resistance (CAMDR) (Hideshima et al., 2007). While the molecular basis underlying CAMDR in MM is still being investigated, interactions between the host microenvironment and numerous other cancer types along with the downstream signaling pathways activated by the interactions remain largely under-explored. Identifying key mediators of tumor cell-ECM interactions and the corresponding downstream signaling pathway(s) that promote(s) cancer progression and resistance to therapy are likely to lead to the development of novel and more efficacious therapeutic strategies that target cancer cells and their microenvironment simultaneously.

Gliomas are the most common type of primary brain cancer and constitute a spectrum of tumors of variable degrees of differentiation and malignancy that may arise from the transformation of neural progenitor cells (Giese et al., 2003; Maher et al., 2001). The most malignant of these tumors is grade IV astrocytoma, also known as glioblastoma multiforme (GBM), which displays highly invasive properties and extremely elevated chemoresistance. Despite aggressive multimodal therapy, GBM remains incurable, with an estimated median survival of less than 1 year and with less than 5% of patients surviving longer than 5 years (Davis et al., 1998). Identification of novel therapeutic targets, development of new agents and novel strategies of combinational treatments to reduce the resistance of GBM to chemo- and established targeted therapies are therefore urgently needed.

The central nervous system contains elevated levels of the broadly distributed glycosaminoglycan hyaluronan (HA); also known as hyaluronic acid or hyaluronan (Park et al., 2008). Gliomas express high levels of a major cell surface HA receptor, CD44, which mediates cell-cell and cell-matrix adhesion and promotes cell migration and signaling (Stamenkovic and Yu, 2009). CD44 is a polymorphic cell surface receptor implicated in diverse cellular functions ((Sherman et al., 1994; Stamenkovic, 2000; Stamenkovic I, 2009; Toole, 2004). It is upregulated in a variety of malignant tumors and its elevated expression correlates with poor prognosis of several cancer types (Lim et al., 2008; Matsumura and Tarin, 1992; Pals et al., 1989; Yang et al., 2008). CD44 is believed to play an important role in the growth and progression of melanoma (Ahrens et al., 2001; Guo et al., 1994) and breast cancer (Yu and Stamenkovic, 1999, 2000; Yu et al., 1997) but little is known about its contribution to the progression of malignant glioma and the responses of GBM cells and other types of cancer cells to chemotherapy and targeted therapies.

CD44 has been shown to be associated with several signaling components and to serve as a co-receptor with several receptor tyrosine kinases (RTKs) (Sherman et al., 1994; Stamenkovic, 2000; Toole, 2004) but no single intact signaling pathway regulated by CD44 has been defined to date. The cytoplasmic domain of CD44 interacts with members of the Band 4.1 superfamily, including ezrin-radixin-moesin (ERM) family proteins (Tsukita and Yonemura, 1997) and merlin (Morrison et al., 2001; Sainio et al., 1997), which serve as linkers between cortical actin filaments and the plasma membrane and regulate actin cytoskeleton organization and cell motility (McClatchey and Giovannini, 2005; Okada et al., 2007). In Drosophila, merlin functions upstream of the Hippo (Hpo) signaling pathway, which plays an important role in restraining cell proliferation and promoting apoptosis in differentiating epithelial cells (Hamaratoglu et al., 2006; Huang et al., 2005; Pellock et al., 2007). The Drosophila hpo gene encodes a serine/threonine kinase that phosphorylates and activates the serine/threonine kinase Warts (Wts). Warts phosphorylates and inactivates a co-transcription factor Yorkie (Yki), which results in repression of a common set of downstream target genes, including dIAP and cyclin E (Hamaratoglu et al., 2006; Huang et al., 2005; Matallanas et al., 2008; Pellock et al., 2007). Although still incompletely characterized, the Hippo pathway is believed to be conserved in mammals where several of its components appear to be tumor suppressors (Lau et al., 2008; Zeng and Hong, 2008). Mammalian homologs of Hpo, Wts, Yki, and dIAP are, respectively, Mammalian Sterile Twenty-like (MST) kinase1 and 2 (MST1/2) (Lehtinen et al., 2006; Ling et al., 2008; Matallanas et al., 2008), Large tumor suppressor homolog 1 and 2 (Lats1 and 2) (Hao et al., 2008; Takahashi et al., 2005), Yes-Associated Protein (YAP) (Overholtzer et al., 2006), and cellular Inhibitor of Apoptosis (cIAP1/2) (Srinivasula and Ashwell, 2008). The upstream components of the mammalian Hippo signaling pathway have not been identified.

Efficacies of current available therapies for many malignant cancers, including glioma, are relative low and render patients with these diseases poor prognosis with short life expectancy after the diagnosis. New targets, agents, and combinational therapeutic approaches for treatment are therefore necessary. In addition, it would be particularly helpful to be able to that target the bulk of tumor cells, including glioma, their stem cells, and their microenvironment simultaneously. The present invention provides such methods.

### SUMMARY OF THE INVENTION

In certain embodiments of the present invention, a method for therapeutic intervention or inhibition of cancer recurrence of a cancer in a mammal is provided, which involves administering to the mammal in need of such treatment an effective amount of a CD44 fusion protein, which includes the constant region of human IgG1 fused to an extracellular domain of CD44, and wherein the cancer is glioma, colon cancer, breast cancer, prostate cancer, ovarian cancer, lung cancer, melanoma, renal cell carcinoma, gastric cancer, esophageal cancer, pancreatic cancer, liver cancer, or head-neck cancer.

In certain aspects of the present invention, a method for treating a cancer in a mammal is provided, which involves administering to the mammal in need of such treatment an effective amount of a CD44 fusion protein, which includes the constant region of human IgG1 fused to an extracellular domain of CD44, and wherein the CD44 fusion protein is administered via a virus carrying an expression vector encoding the CD44 fusion protein and, optionally, a pharmaceutically acceptable carrier or diluent.

In certain aspects of the present invention, a method for treating a cancer in a mammal is provided, which involves administering to the mammal in need of such treatment an effective amount of a CD44 fusion protein, which includes the constant region of human IgG1 fused to an extracellular domain of CD44, and wherein the CD44 fusion protein is administered as purified protein and b) a pharmaceutically acceptable carrier or diluent.

In certain aspects of the present invention, a method for treating a cancer in a mammal is provided, which involves administering to the mammal in need of such treatment an effective amount of a CD44 fusion protein, which includes the constant region of human IgG1 fused to an extracellular domain of CD44, and wherein the extracellular domain of CD44 is CD44s, CD44v3-v10, CD44v8-v10, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, CD44sR41A, CD44v3-v10R41A, CD44v8-v10R41A, CD44v4-v10R41A, CD44v5-v10R41A, CD44v6-v10R41A, CD44v7-v10R41A, CD44v9-v10R41A, CD44v10R41A, CD44v9R41A, CD44v8R41A, CD44v7R41A, CD44v6R41A, CD44v5R41A, CD44v4R41A, or CD44v3R41A.

In other embodiments of the present invention, a pharmaceutical composition is provided, which includes: a) a CD44 fusion protein comprising the constant region of human IgG1 fused to an extracellular domain of CD44, wherein the extracellular domain of CD44 is CD44v3-v10, CD44v8-v10, CD44s, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, CD44sR41A, CD44v3-v10R41A, CD44v8-v10R41A, CD44v4-v10R41A, CD44v5-v10R41A, CD44v6-v10R41A, CD44v7-v10R41A, CD44v9-v10R41A, CD44v10R41A, CD44v9R41A, CD44v8R41A, CD44v7R41A, CD44v6R41A, CD44v5R41A, CD44v4R41A, or CD44v3R41A; and b) a pharmaceutically acceptable carrier or diluent.

In other aspects of the present invention, a method for treating a cancer in a mammal is provided, which involves administering to the mammal in need of such treatment an effective amount of a CD44 fusion protein comprising the constant region of human IgG1 fused to an extracellular domain of CD44 along with one or more additional anti-cancer therapies.

In certain aspects of the present invention, the additional anti-cancer therapies are surgery, chemotherapy, radiation therapy, targeted therapy, and immunotherapy. In certain aspects of the present invention, the additional anti-cancer therapy is radiation therapy. In other aspects of the present invention, the additional anti-cancer therapy is surgery.

In certain aspects of the present invention, the additional anti-cancer therapy is chemotherapy. In certain aspects of the present invention, the chemotherapy is temozolomide, carmustine, docetaxel, carboplatin, cisplatin, epirubicin, oxaliplatin, cyclophosphamide, methotrexate, fluorouracil, vinblastine, vincristine, mitoxantrone, satraplatin, ixabepilone, pacitaxel, gemcitabine, capecitabine, doxorubicin, etoposide, melphalan, hexamethylamine, irinotecan, or topotecan. In another aspect of the present invention, the chemotherapy is temozolomide or carmustine.

In certain aspects of the present invention, the additional anti-cancer therapy is targeted therapy. In certain embodiment of the present invention, the targeted therapy is a receptor tyrosine inhibitor. In certain embodiment of the present invention, the targeted therapy is an inhibitor of erbB receptor. In certain embodiment of the present invention, the targeted therapy is an inhibitor of c-Met. In certain embodiment of the present invention, the targeted therapy is an inhibitor of VEGFR. In certain embodiment of the present invention, the targeted therapy is an agent that promotes apoptosis and stress responses. In certain embodiment of the present invention, the targeted therapy is an inhibitor of the Wnt signaling pathway. In certain embodiments of the present invention, the targeted therapy is Trastuzumab, cetuximab, panitumumab, gefitinib, erlotinib, lapatinib, BIBW2992, CI-1033, PF-2341066, PF-04217903, AMG 208, JNJ-38877605, MGCD-265, SGX-523, GSK1363089, sunitinib, sorafenib, vandetanib, BIBF1120, pazopanib, bevacizumab, vatalanib, axitinib, E7080, perifosine, MK-2206, temsirolimus, rapamycin, BEZ235, GDC-0941, PLX-4032, imatinib, AZD0530, bortezomib, XAV-939, advexin (Ad5CMV-p53), Genentech—Compound 8/cIAP-XIAP inhibitor, or Abbott Laboratories—Compound 11.

In other embodiments of the present invention, a pharmaceutical composition is provided, which includes: a) a CD44 fusion protein comprising the constant region of human IgG1 fused to an extracellular domain of CD44, wherein the extracellular domain of CD44 is a CD44v3-v10, CD44v8-v10, CD44s, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, CD44sR41A, CD44v3-v10R41A, CD44v8-v10R41A, CD44v4-v10R41A, CD44v5-v10R41A, CD44v6-v10R41A, CD44v7-v10R41A, CD44v9-v10R41A, CD44v10R41A, CD44v9R41A, CD44v8R41A, CD44v7R41A, CD44v6R41A, CD44v5R41A, CD44v4R41A, or CD44v3R41A; b) at least one therapeutic agent that causes cytotoxic or cytostatic stress in cancer cells; and c) a pharmaceutically acceptable carrier or diluent.

In another embodiments of the present invention, a pharmaceutical composition is provided, which includes: a) a CD44 fusion protein comprising the constant region of human IgG1 fused to an extracellular domain of CD44, wherein the extracellular domain of CD44 is a CD44v3-v10, CD44v8-v10, CD44s, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, CD44sR41A, CD44v3-v10R41A, CD44v8-v10R41A, CD44v4-v10R41A, CD44v5-v10R41A, CD44v6-v10R41A, CD44v7-v10R41A, CD44v9-v10R41A, CD44v10R41A, CD44v9R41A, CD44v8R41A, CD44v7R41A, CD44v6R41A, CD44v5R41A, CD44v4R41A, or CD44v3R41A; b) at least one therapeutic agent that inhibits EGFR/erbB-2/erbB-3/erbB-4/c-Met/VEGFR RTK in cancer cells; and c) a pharmaceutically acceptable carrier or diluent. In certain aspects of the present invention, the inhibitor of EGFR/erbB-2/erbB-4/c-Met/VEGFR RTK includes Trastuzumab, cetuximab, panitumumab, gefitinib, erlotinib, lapatinib, BIBW2992, CI-1033, PF-2341066, PF-04217903, AMG 208, JNJ-38877605, MGCD-265, SGX-523, GSK1363089, sunitinib, sorafenib, vandetanib, BIBF1120, pazopanib, bevacizumab, vatalanib, axitinib, and E7080.

In another embodiments of the present invention, a pharmaceutical composition is provided, which includes: a) a CD44 fusion protein comprising the constant region of human IgG1 fused to an extracellular domain of CD44, wherein the extracellular domain of CD44 is a CD44v3-v10, CD44v8-v10, CD44s, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, CD44sR41A, CD44v3-v10R41A, CD44v8-v10R41A, CD44v4-v10R41A, CD44v5-v10R41A, CD44v6-v10R41A, CD44v7-v10R41A, CD44v9-v10R41A, CD44v10R41A, CD44v9R41A, CD44v8R41A, CD44v7R41A, CD44v6R41A, CD44v5R41A, CD44v4R41A, or CD44v3R41A; b) at least one therapeutic agent that inhibits IAPs or promotes stresses in cancer cells; and c) a pharmaceutically acceptable carrier or diluent. In certain aspects of the present invention, the inhibitor of IAPB or promotes stresses includes advexin (Ad5CMV-p53), Genentech—Compound 8/cIAP-XIAP inhibitor, Abbott Laboratories—Compound 11, perifosine, MK-2206, temsirolimus, rapamycin, BEZ235, GDC-0941, PLX-4032, imatinib, AZD0530, bortezomib, or XAV-939.

In other embodiments of the present invention, a pharmaceutical composition is provided, which includes: a) a virus carrying a expression vector encoding a CD44 fusion protein comprising the constant region of human IgG1 fused to an extracellular domain of CD44, wherein the extracellular domain of CD44 is CD44v3-v10, CD44v8-v10, CD44s, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, CD44sR41A, CD44v3-v10R41A, CD44v8-v10R41A, CD44v4-v10R41A, CD44v5-v10R41A, CD44v6-v10R41A, CD44v7-v10R41A, CD44v9-v10R41A, CD44v10R41A, CD44v9R41A, CD44v8R41A, CD44v7R41A, CD44v6R41A, CD44v5R41A, CD44v4R41A, or CD44v3R41A; and b) a pharmaceutically acceptable carrier or diluent.

In certain aspects of the above embodiments, the cancer is glioma, colon cancer, breast cancer, prostate cancer, ovarian cancer, lung cancer, melanoma, renal cell carcinoma, gastric cancer, esophageal cancer, pancreatic cancer, liver cancer or head-neck cancer. In certain embodiments of the present invention the glioma is an astrocytoma. In other embodiments of the present invention the glioma is a glioblastoma multiforme. In certain embodiments of the present invention the mammal is a human.

In certain aspects of the above embodiments, the extracellular domain of the CD44 is CD44v3-v10. In other aspects of the above embodiments, the extracellular domain of the CD44 is CD44v8-v10. In another aspect of the above embodiments, the extracellular domain of the CD44 is CD44s. In another aspect of the above embodiments, the extracellular domain of the CD44 is CD44v6-v10.

In other embodiments of the present invention, methods of detecting hyaluronan in a sample are provided, comprising contacting the sample with a labeled CD44 fusion protein comprising the constant region of human IgG1 fused to an extracellular domain of CD44. In certain embodiments, the sample is a cancer biopsy or cancer section. In other embodiments, the sample is a patient fluid sample is blood, serum, plasma, or urine. In other embodiments, the label is biotin, fluorescent labels, alkaline phosphatase, horseradish peroxidase, magnetic beads, or radioactive labels. In yet another embodiment, the methods further comprise incubating the labeled CD44 fusion protein in the sample and quantifying the label bound to hyaluronan. In yet another embodiment, the extracellular domain of CD44 is CD44v3-v10, CD44v8-v10, CD44s, CD44v4-v10, CD44v5-v10, CD44v6-v10, CD44v7-v10, CD44v9-v10, CD44v10, CD44v9, CD44v8, CD44v7, CD44v6, CD44v5, CD44v4, or CD44v3.

In other embodiments of the present invention, methods of diagnosing a cancer in a mammal are provided, comprising detecting hyaluronan in a sample from the mammal, wherein the detecting is done according to any of the described methods of detecting hyaluronan, and wherein an increase in the amount of hyaluronan in the sample compared to a normal control sample indicates the presence of cancer. In certain embodiments, the cancer is a glioma, colon cancer, breast cancer, prostate cancer, ovarian cancer, lung cancer, renal cell carcinoma, gastric cancer, esophageal cancer, head-neck cancer, pancreatic cancer, or melanoma. In certain other embodiments, the methods further comprise detecting CD44 in the sample, and wherein an increase in the amount of hyaluronan and CD44 in the sample compared to a normal control sample indicates the presence of cancer.

In other embodiments of the present invention, methods of determining a change in the cancerous state of a mammal are provided, comprising collecting a first sample from the mammal, detecting hyaluronan in the first sample from the mammal, wherein the detecting is done according to any of the described methods of detecting hyaluronan, collecting a second sample from the mammal, detecting hyaluronan in a second sample from the mammal, wherein the detecting is done according to any of the described methods of detecting hyaluronan, wherein a difference in the amount of hyaluronan in the second sample compared to the amount in the first sample indicates a change in the cancerous state of the mammal.

These and others aspects of the present invention will be apparent to those of ordinary skill in the art in light of the present specification, claims, and drawings.

### BRIEF DESCRIPTION OF THE DRAWINGS

FIG. 1A: Bar graph showing the up-regulation of expression of CD44 transcripts in microarray data sets (derived from http://www.oncomine.org/) of multiple glioma tissues compared to normal human brain tissues (study 1, 2, and 4) or to normal white matter (study 3). CD44 expression in brains or white matter from epilepsy patients (bars on the left of each study) or in GBMs (bars on the right of each study) are shown.

FIG. 1B: Representative pictures of CD44 immunohistochemistry performed on 14 GBM tissues (B-a) and 8 normal human brain samples (B-b) using anti-CD44 antibody (Santa Cruz). Bar, 50 μm.

FIG. 1C: Western Blot of endogenous CD44 expression in a panel of human glioma cells using anti-CD44 antibody (Santa Cruz). Proteins from normal human astrocytes (NHAs, ALLCELLS, Inc.) were loaded in lanes 1 and 20. Actin was included as an internal control for loading (lower panels). The molecular weight bars correspond to 197 kDa, 110 kDa, and 72 kDa.

FIG. 2A: Western blot of CD44 expression knockdown by lentiviral based shRNAs in U251 (A-a) or U87MG (A-b) cells using anti-CD44 mAb (Santa Cruz).

FIG. 2B: Immunocytochemistry shows reduced endogenous CD44 levels in U87MG cells infected with different CD44 knockdown shRNA lentiviral vectors (b-c) comparing to U87MG cells infected with non-targeting (TRC-NT) control shRNA (a) using anti-CD44 mAb (Santa Cruz).

FIG. 2C: Fluorescent-HA (FL-HA) binding assay with wild type U87MG cells (C-a), U87MG cells infected with TRC-NT control shRNA (C-c), and U87MG cells infected with different CD44 knockdown shRNAs lentiviral vectors (C-b, -d) as indicated in the panels.

FIG. 2D: Immunocytochemistry of endogenous CD44 levels in U251 cells infected with different CD44 knockdown shRNA lentiviruses (a-d) or TRC-NT control shRNA lentiviruses (e) using anti-CD44 mAb (Santa Cruz).

FIG. 3A: Bar graph showing that knockdown of CD44 expression inhibits subcutaneous growth of U87MG glioma cells in vivo.

FIG. 3B: Bar graph showing that knockdown of CD44 expression inhibits subcutaneous growth of U251 glioma cells in vivo.

FIG. 3C: Line graph showing the in vivo growth rates of the subcutaneous tumors derived from U87MG cells infected with different shRNA constructs.

FIG. 3D: Line graph showing the in vivo growth rates of the subcutaneous tumors derived from U251 cells infected with different shRNA constructs.

FIG. 3E: Morphology (H&E), proliferation (Brdu and Ki67) and apoptosis (Apoptag) status of subcutaneously explanted glioma tumors.

FIG. 4A: Bioluminescence imaging analysis of mice 3, 6, 9, and 13 days following the intracranial injection of U87MG-TRC-NT, U87MGshRNAmir-NT (non-targeting shRNA controls, upper panels), and U87MG-TRC-CD44#3 and U87MGshRNAmir-CD44#1 (shRNAs against human CD44, bottom panels).

FIG. 4B: Line graph showing the survival rates of mice following intracranial injections of the transduced U87MG (B-a) and U251 (B-b) cells as detailed in the panels.

FIG. 4C: Bioluminescence imaging analysis of mice 6, 9, 13, and 17 days following intracranial injection of U87MG-NT (U87MG cells infected with a mixture of lentiviruses carrying non-targeting TRC-NT and shRNAmir-NT constructs), U87MGshRNA-CD44 (U87MG cells infected with a mixture of lentiviruses carrying TRC-CD44#3 and shRNAmir-CD44#1 constructs, which effectively knock down CD44 expression). These mice were treated with or without chemotherapeutic agents (BCNU or TMZ) as detailed in the panels.

FIG. 4D: Line graph showing the survival rates of mice following intracranial injections of the transduced U87MG (D-a) and U251 (D-b) cells with or without CD44 knockdown. These mice were treated with or without chemotherapeutic agents (BCNU or TMZ) as detailed in the panels.

FIG. 5A: Western blot analysis of the levels of phosphorylated and/or total merlin, MST1/2, Lats1/2, YAP, cIAP1/2, and cleaved caspase 3 induced by oxidative stresses (H₂O₂) in U87MG cells infected with a mixture of lentiviruses carrying non-targeting TRC-NT and shRNAmir-NT constructs.

FIG. 5B: Western blot analysis of the levels of phosphorylated and/or total merlin, MST1/2, Lats1/2, YAP, cIAP1/2, and cleaved caspase 3 induced by oxidative stresses in U87MG cells infected with a mixture of lentiviruses carrying shRNAs against human CD44, TRC-CD44#3 and shRNAmir-CD44#1, which resulted in effective knockdown of CD44 in these cells.

FIG. 5C: Western blot analysis of the levels of phosphorylated and/or total JNK and p38 stress kinases, p53, p21, and puma induced by oxidative stresses in U87MG cells infected with a mixture of lentiviruses carrying non-targeting TRC-NT and shRNAmir-NT constructs.

FIG. 5D: Western blot analysis of the levels of phosphorylated and/or total JNK and p38 stress kinases, p53, p21, and puma in U87MG cells infected with a mixture of lentiviruses carrying shRNAs against human CD44, TRC-CD44#3 and shRNAmir-CD44#1.

FIG. 6A: Western blot analysis of the levels of phosphorylated and/or total MST1/2, YAP, cIAP1/2, JNK and p38 stress kinases, p53, and p21 induced by a chemotherapeutic agent, TMZ, in U87MG cells infected with a mixture of lentiviruses carrying non-targeting TRC-NT and shRNAmir-NT constructs.

FIG. 6B: Western blot analysis of the levels of phosphorylated and/or total MST1/2, YAP, cIAP1/2, JNK and p38 stress kinases, p53, and p21 induced by a chemotherapeutic agent, TMZ, in U87MG cells infected with a mixture of lentiviruses carrying shRNAs against human CD44, TRC-CD44#3 and shRNAmir-CD44#1.

FIG. 7A: Western blot of the activation of Erk1/2 kinases induced by the ligands of erbB and c-Met receptor tyrosine kinase (RTK) in U87MG cells infected with a mixture of lentiviruses carrying non-targeting TRC-NT and shRNAmir-NT constructs.

FIG. 7B: Western blot of the activation of Erk1/2 kinases induced by the ligands of erbB and c-Met receptor tyrosine kinase (RTK) in U87MG cells infected with a mixture lentiviruses carrying shRNAs against human CD44, TRC-CD44#3 and shRNAmir-CD44#1.

FIG. 8. The signaling pathways that are significantly affected by increased expression of merlin, the downstream CD44 effector that is negatively regulated by CD44, in U87MG human glioma and WM793 human melanoma cells. Functional analysis of the data sets from microarray experiments is shown. The data indicates that increased expression of merlin activates Hippo and inhibits Wnt and c-Met signaling pathways.

FIG. 9. Merlin inhibits canonical Wnt signaling in human glioma cells. A-B, Luciferase activity was measured in U87MGwt, U87MGmerlin, U87MGmerlinS518D, and U87MGmerlinS518A cells 24 hours after transfection of cells with TopFlash (A) or FopFlash (B) in triplicates.

FIG. 10. A model of merlin-mediated signaling events and their potential cross-talk. The components of Drosophila Hippo signaling pathway are underlined. Merlin functions upstream of the mammalian Hippo (merlin-MST1/2-LATS1/2-YAP) and JNK/p38 signaling pathways and plays an essential role in regulating the cell response to the stresses and stress-induced apoptosis as well as to proliferation/survival signals. CD44 functions upstream of merlin and Hippo signaling pathway. Merlin and CD44 antagonize each other's function. Merlin inhibits activities of RTKs and the RTK-derived growth and survival signals. CD44 function upstream of mammalian Hippo signaling pathway and enhances activities of RTKs and Wnt signaling.

FIG. 11. Biochemical and functional properties of hsCD44-Fc and hsCD44R41A-Fc fusion proteins. A, Western blot analysis of serum-free cell culture supernatants derived from U251 cells transduced with retroviruses carrying the expression constructs of hsCD44s-Fc, hsCD44v8-v10-Fc, hsCD44v3-v10-Fc, hsCD44sR41A-Fc, hsCD44v8-v10R41A-Fc, hsCD44v3-v10R41A-Fc, or the empty expression vector. Anti-CD44 antibody (Santa Cruz) was used to detect the fusion proteins. The molecular weight bars correspond to 199 kDa and 116 kDa. B, FL-HA binding assays were performed. U251 cells expressing hsCD44s-Fc, hsCD44sR41A-Fc, or infected with retroviruses carrying the empty expression vectors were cultured for two days. FL-HA (20 μg/ml) was added into culture media and the cells were cultured additional 12 h before fixing the cells. Bar, 40 μm. C, hsCD44v3-v10-Fc proteins are modified by heparan sulfate (HS). Purified hsCD44s-Fc, hsCD44v8-v10-Fc, and hsCD44v3-v10-Fc fusion proteins were treated with or without heparinase VIII before eluting from protein A columns. These proteins were bound onto Elias plates in triplicate. After blocking with BSA, the coated proteins reacted with anti-HS antibody (Calbiochem). The intensity of the reaction color was measure by an Elisa reader and normalized by the reactivity to anti-CD44 antibody, which provides relative quantity of the coated fusion proteins on the plates.

FIG. 12. Antagonists of CD44 are effective therapeutic agents against human GBM in mouse models. A, U87MG and U251 cells were transduced with the retroviruses carrying empty vector and the expression constructs of hsCD44s-Fc, hsCD44v8-v10-Fc, and hsCD44v3-v10 and their serum free cell culture supernatants were collected and analyzed on Western blots using anti-CD44 antibody (Santa Cruz) or anti-human IgG antibody. The molecular weight bars correspond to 199 kDa and 116 kDa. B, 2×10⁶ of the transduced U87MG and U251 cells were injected subcutaneously per mouse. Growth rates of the subcutaneous tumors were determined and expressed as the mean of tumor volume (mm³)+/−SD. Six mice were used for each construct. C, Survival rates of mice following the intracranial injections of transduced U87MG (C-a) and U251 (C-b-c) cells infected with the retroviruses carrying the empty expression vectors, hsCD44s-Fc, hsCD44v8-v10-Fc, hsCD44v3-v10, hsCD44sR41A-Fc, or hsCD44v3-v10R41A-Fc constructs as indicated in the panels. 15 mice were used for each type of transduced glioma cells in a-b and ten mice were used in panel c.

FIG. 13. Purified hsCD44s-Fc fusion proteins inhibit intracranial glioma growth in Rag-1 mice and display an intra-tumor distribution pattern without apparent toxicity. A-B, Treatment of pre-established intracranial U87MG (A) and U251 (B) gliomas with intravenous delivery of 5 mg/kg purified hsCD44s-Fc fusion proteins or human IgG every third day. The results show that hsCD44s-Fc but not human IgG significantly extended the survival of the experimental mice (p<0.001). Six Rag-1 mice were used for each treatment. C, Distribution of hsCD44s-Fc fusion proteins in intracranial gliomas (C-b and C-d) and normal adjacent brain tissues (C-a, and C-c). The fusion proteins were detected by anti-human IgG antibody. Bar in a and b, 100 μm and in c and d, 50 μm. D, Purified CD44s-Fc fusion protein displayed no apparent toxicity towards normal host tissues. H&E staining of normal tissues derived from the Rag-1 mice received iv injection of 5 mg/kg of hsCD44s-Fc fusion proteins (right side panels) or human IgG (left side panels) every third day. Bar, 50 μm.

FIG. 14. A. Glioma cell viability assays: knockdown of human CD44 sensitizes the response of GBM cells to a dual EGFR/erbB-2 inhibitor, BIBW2992. B. Glioma cell viability assays: knockdown of human CD44 sensitizes the response of GBM cells to a pan EGFR/erbB-2/erbB-4 inhibitor, CI-1033. C. Glioma cell viability assays: knockdown of human CD44 sensitizes the response of GBM cells to a c-Met inhibitor, SU11274.

FIG. 15. hsCD44-Fc fusion proteins sensitize the responses of GBM cells to chemotherapeutic and targeted agents. Glioma cell viability assays were performed using the Cell Titer-Glo Luminescent Cell Viability Assay kit (Promega) following manufacturer's instruction. U87MG cells were plated in triplicate at 1×10⁵ cells per well treated different concentrations of TMZ (A), gefitinib (B), BIBW2992 (C), CI-1033 (D), or PF-2341066 (E) as detailed in the panels in the presence or absence of hsCD44s-Fc fusion proteins or human IgG (10 μg/ml) for 48 hours before the cell viability was measured.

FIG. 16. hsCD44s-Fc fusion protein displayed low cytotoxicity toward a panel of normal cells. Cell viability assays were performed as described in FIG. 15. NHAs, normal human Schwann cells, HUVECs, normal fibroblasts, and U251 GBM cells were plated in triplicate at 1×10⁵ cells/well in 96 well plates and treated different concentrations of purified hsCD44s-Fc fusion proteins for 48 hours before the cell viability was measured.

FIG. 17. Establishment and characterization of primary human glioma spheres (GBM stem cells, GBMCSCs) from fresh GBM tissues. A, Self-renewal capacity of glioma spheres. A-a, single primary GBMCSCs were maintained in serum free stem cell medium (SCCM). Small (b), intermediate (c), and large (d) GBM spheres were formed after culturing for 2-3, 4-5, and 6-7 days in SCCM. B, The glioma spheres were disaggregated and cultured in SCCM for 12 hours and stained positive for the glioma stem cell markers, nestin (B-a) or Sox2 (B-b). Another set of the cells were cultured in astrocyte medium (ScienCell) for 6 day before stained positive for a differentiated astrocyte specific marker, glial fibrillary acidic protein (GFAP, B-c). In panel B-d, only second antibody was used as a control showing the absence of non-specific staining. Bar, 150 μm. C, human glioma spheres (HGSs), MSSM-GBMCSC-1, were disaggregated and seeded on the BD BioCoat™ Matrigel™ Matrix 6-well plates, which were designed to maintain and propagate embryonic stem cells in the absence of feeder layers. These cells were transduced with retroviruses carrying GFP. After selection with puromycin, the pooled populations of drug-resistant cells were suspended into single cells and culture in SCCM in ultra-low attachment plates to re-form spheres. GFP expression by the re-formed spheres (C-a), morphology (C-b) and merged pictures (C-c) of these spheres are shown. Bar: 300 μm. D, MSSM-GBMCSC-1 cells form invasive intracranial tumors ˜25 days after injection of 5×10⁴ of the cells (D-a) and overexpression of hsCD44s-Fc fusion proteins inhibits intracranial growth of MSSM-GBMCSC-1 cells (D-b).

FIG. 18. Morphology of glioma sphere cells, MSSM-GBMCSC-1, derived from a GBM patient showing that knockdown of CD44 expression by a mixture of lentiviruses carrying shRNAs against human CD44, TRC-CD44#3 and shRNAmir-CD44#1, inhibits the formation of glioma spheres.

FIG. 19. CD44 expression. A: Bar graph showing that CD44 mRNA expression level is up-regulated in human colon cancer (right side of each study) comparing to normal colon (left side of each study) (data derived from http://www.oncomine.org/). B: Representative pictures of CD44 immunohistochemistry performed on 6 normal colon tissue samples (B-a), 6 malignant colon cancer samples (B-b), and 6 liver metastasis of colon cancers (B-c) using anti-CD44 antibody (Santa Cruz). C: Additional bar graphs showing that CD44 mRNA expression level is up-regulated in human colon cancer (right side of each study) comparing to normal colon (left side of each study) (data derived from http://www.oncomine.org/).

FIG. 20. A: Western blot of CD44 expression knockdown in HCT116 human colon cancer cells transduced with shRNAs against human CD44 or non-targeting (NT) shRNAs using anti-CD44 antibody (Santa Cruz). B: Bar graph showing that knockdown of CD44 expression inhibits subcutaneous growth of HCT116 human colon cancer cells in vivo. HCT116 cells were transduced with shRNAs against human CD44 or non-targeting (NT) shRNAs. (n=6)

FIG. 21A. Western blot of CD44 expression knockdown in KM20L2 human colon cancer cells transduced with shRNAs against human CD44 or non-targeting (NT) shRNAs using anti-CD44 antibody (Santa Cruz). 21B: Bar graph showing that knockdown of CD44 expression inhibits subcutaneous growth of KM20L2 human colon cancer cells in vivo. KM20L2 cells were transduced with shRNAs against human CD44 or non-targeting (NT) shRNAs. (n=6)

FIG. 22. Representative pictures of CD44 immunohistochemistry performed on 6 normal prostate tissue samples (A) and 6 malignant prostate cancer samples (B) using anti-CD44 antibody (Santa Cruz), showing that CD44 is up-regulated in malignant prostate cancer.

FIG. 23. A: Western blot of CD44 expression knockdown in PC3/M human prostate carcinoma cells transduced with shRNAs against human CD44 or non-targeting (NT) shRNAs using anti-CD44 antibody (Santa Cruz). 23B: Bar graph showing that knockdown of CD44 expression inhibits subcutaneous growth of PC3/M human prostate carcinoma cells in vivo. PC3/M cells were transduced with shRNAs against human CD44 or non-targeting (NT) shRNAs (n=6).

FIG. 24. CD44-Fc fusion proteins are effective therapeutic agents against human prostate cancer cells in vivo. A, expression of CD44 by human prostate cancer cells were assessed by Western blotting using anti-CD44 antibody (Santa Cruz). B, 5×10⁶ PC3/M cells were injected subcutaneously into each Rag-1 mice. The tumors were allowed to grow for ˜two weeks when tumor volumes reach ˜150 mm³. The mice bearing similar size tumors were separated into 6 groups (6mice/group) and treated every other days with 4 intratumoral injections of 5 μl/injection of 10 mg/ml of hsCD44s-Fc, hsCD44v8-v10-Fc, hsCD44v6-v10-Fc, hsCD44v3-v10-Fc, or human IgG, or 0.9% NaCl. The experiments were stopped when tumors of control groups (treatment of human IgG or 0.9% NaCl) reached ˜1 cm in their longest diameters. All the tumors were dissected out and weighted. Data is presented as the mean of tumor weight+/−SD.

FIG. 25. Human malignant breast cancer cells that infiltrated host stroma expresses a high level of CD44 and breast cancer stroma accumulates a high level of hyaluronan (HA). Expression of CD44 protein (A-C) is up-regulated in malignant breast cancer cells that infiltrated stroma (B-C) compared to normal human breast epithelia (A) as assessed by immunohistochemistry using anti-CD44 antibody (Santa Cruz). In addition, a much higher level of HA is accumulated in breast cancer stroma (E) compared to normal breast stroma (D). HA was detected by biotinylated hsCD44-Fc fusion proteins. Representative images from 6 normal and 6 malignant breast cancer tissues are shown. Bar in A, C-E, 50 μm and in B, 200 μm.

FIG. 26A: Western blot of CD44 expression knockdown in MX-2 human breast carcinoma cells transduced with shRNAs against human CD44 or non-targeting (NT) shRNAs using anti-CD44 antibody (Santa Cruz). 26B: Bar graph showing that knockdown of CD44 expression inhibits subcutaneous growth of MX-2 human breast carcinoma cells in vivo. MX-2 cells were transduced with shRNAs against human CD44 or non-targeting (NT) shRNAs (n=6).

FIG. 27. A: Western blot of CD44 expression knockdown in SW613 human breast carcinoma cells transduced with shRNAs against human CD44 or non-targeting (NT) shRNAs using anti-CD44 antibody (Santa Cruz). B: Bar graph showing that knockdown of CD44 expression inhibits subcutaneous growth of SW613 human breast carcinoma cells in vivo. SW613 cells were transduced with shRNAs against human CD44 or non-targeting (NT) shRNAs (n=6).

FIG. 28. Establishment and characterization of human breast cancer stem cells (BCSCs) and in vivo xenograft breast cancer models. A, CD44 expression by normal human mammary epithelial cells (line 1), MSSM-BCSC-1, -2, and -3 (lane 2-4) was determined by western blots using anti-CD44 mAb. Actin levels were used as loading controls (the bottom panel). B, The BCSCs express high levels of the stem cells markers as assessed by immunocytochemistry using antibodies against CD44, Sox-2, Oct3/4, and SSEA1 and they express a low level of CD24. Bar, 100 μm. C-a-c, Self-renewal capacity of the mammospheres. C-a, single primary BCSCs were maintained in serum free stem cell medium (SCCM). Intermediate (C-b) and large (C-c) mammospheres were formed after culturing for 4-5, and 6-7 days in SCCM. C-d-f, Morphology of mammospheres showing that knockdown of CD44 expression in BCSCs inhibits the sphere formation (f) whereas non-targeting shRNAs have no effect (e) when compared to the parental BCSCs (d). Bar, 200 μm. D, Quantitative analysis revealed the inhibitory effect of CD44 knockdown on the sphere formation. The numbers of spheres were counted in ten randomly selected 100× microscopic field, averaged, and presented as the means+/−SD. E, Bioluminescence images of subcutaneous tumors derived from MSSM-BCSCs.

FIG. 29. Antagonists of CD44, hsCD44v3-v10-Fc, hsCD44v6-v10-Fc, hsCD44v8-v10-Fc, and hsCD44s-Fc fusion proteins, are effective therapeutic agents against human breast cancer stem cells in vivo. A, analysis of purified hsCD44s-Fc, hsCD44v8-v10-Fc, hsCD44v6-v10, and hsCD44v3-v10 by Western blotting using anti-CD44 antibody (Santa Cruz). B, 1×10⁶ MSSM-BCSC-1 cells were injected subcutaneously into each Rag-1 mice. The tumors were allowed to growth for ˜three weeks when the tumor volumes reach ˜200 mm³. The mice bearing similar size tumors were separated into 6 groups (6mice/group) and were treated every other days with 4 intratumoral injections of 5 μl/injection of 10 mg/ml of hsCD44s-Fc, hsCD44v8-v10-Fc, hsCD44v6-v10-Fc, hsCD44v3-v10-Fc, or human IgG, or 0.9% NaCl. The experiments were stopped when tumors of the control groups (treatment of human IgG or 0.9% NaCl) reached ˜1 cm in their longest diameters. All the tumors were dissected out and weighted. Data is presented as the mean of tumor weight+/−SD.

FIG. 30. A: Western blot of CD44 expression knockdown in NCI-H125 human lung cancer cells transduced with shRNAs against human CD44 or non-targeting (NT) shRNAs using anti-CD44 antibody (Santa Cruz). B: Bar graph showing that knockdown of CD44 expression inhibits subcutaneous growth of NCI-H125 human lung cancer cells in vivo. NCI-H125 cells were transduced with shRNAs against human CD44 or non-targeting (NT) shRNAs (n=6).

FIG. 31. A: Western blot of CD44 expression knockdown in NCI-H460 human lung cancer cells transduced with shRNAs against human CD44 or non-targeting (NT) shRNAs using anti-CD44 antibody (Santa Cruz). B: Bar graph showing that knockdown of CD44 expression inhibits subcutaneous growth of NCI-H460 human lung cancer cells in vivo. NCI-H460 cells were transduced with shRNAs against human CD44 or non-targeting (NT) shRNAs. (n=6)

FIG. 32. A: Bar graph showing that CD44 mRNA expression level is up-regulated in human ovarian cancer (right side of each study) comparing to normal ovary (left side of each study) (data derived from http://www.oncomine.org/). B-D. Stroma of stage III/IV ovarian cancer tissues express high levels of CD44 (C) and/or hyaluronan (HA, D) compared to normal ovary (B). Levels of CD44 protein and HA were assessed by immunohistochemistry using anti-CD44 antibody (Santa Cruz, B and C) and biotinylated hsCD44-Fc (D), respectively. OSE: ovary surface epithelial cells. Bar, 50 μm in C and 100 μm in A-B.

FIG. 33. A: Western blot of CD44 expression knockdown in OVCAR-3 human ovarian cancer cells transduced with shRNAs against human CD44 or non-targeting (NT) shRNAs using anti-CD44 antibody (Santa Cruz). B: Bar graph showing that knockdown of CD44 expression inhibits subcutaneous growth of OVCAR-3 human ovarian cancer cells in vivo. OVCAR-3 cells were transduced with shRNAs against human CD44 or non-targeting (NT) shRNAs (n=6).

FIG. 34. Establishment of ovarian cancer stem cells (OCSCs, B-C, E) and in vivo ascites tumor models (A). B, Positive expression of stem cell markers are shown as assessed by immunocytochemistry using antibodies against CD44 (B-a), Sox-2 (B-b), Oct3/4 (B-c), and Nanog (B-d). Bar, 50 μm. C, Formation of ascites tumors in Rag-1 mice by MSSM-OCSC1 cells: MSSM-OCSC1 cells formed tumors that attached to peritoneal wall (C-a), liver (C-b), and mesentery (C-c). Bar, 150 μm. D, Western blot analysis of CD44 expression in MSSM-OCSC1 cells transduced with shRNAs against CD44 (lane 3) or non-targeting shRNAs (lane 2). CD44 level in parental cells is shown in lane 1. MSSM-OCSCs express several CD44 variants and the standard form of CD44, CD44s (the lower band). E-a-c, Self-renewal capacity of MSSM-OCSC-1 spheres. The suspended OCSCs were maintained in serum free stem cell medium for 2-3 (a), 4-5 (b), and 6-7 (c) days. E-d-f, CD44 is required for self-renewal of OCSCs: Knockdown of CD44 (D-f) but not parental (D-d) or non-targeting shRNA (D-e) inhibits the formation of OCSC spheres. F, Quantitative analyses of D-d-f are shown. The numbers of spheres in twenty randomly selected 100× microscopic fields were counted, averaged, and presented as the means+/−SD.

FIG. 35. CD44 expression is up-regulated in human melanomas (A-B) and melanoma cells (C). A-B, CD44 mRNA levels in Talantov Melanoma data set (www.oncomine.org). C, CD44 expression in human melanocytes and melanoma cells was assessed by Western blotting using anti-CD44 antibody.

FIG. 36. hsCD44-Fc fusion proteins inhibit human melanoma growth in vivo. A, Overexpression of hsCD44s-Fc, hsCD44v8-v10-Fc, and hsCD44v3-v10-Fc fusion proteins by human M14 melanoma cells (lane 1-3). B, 5×10⁶ of M14 cells expressing different CD44-Fc fusion proteins or transduced with empty expression vectors were injected subcutaneously into each Rag-1 mice. Tumors were allowed to grow for ˜four weeks. At the end of experiments, all the tumors were dissected out and weighted. Data is presented as the mean of tumor weight (gram)+/−SD.

FIG. 37. CD44 mRNA level is up-regulated in human clear cell sarcoma and renal cell carcinoma (right side of the studies) comparing to normal fetal kidney (left side of the studies). Data derived from oncomine (www.oncomine.org).

FIG. 38. CD44 mRNA level is up-regulated in human Head and Neck Squamous carcinoma (A, right side of the study) and renal cell carcinoma (B, right side of the study comparing to normal oral mucosa (left side of A) or normal kidney tissues (left side of B). Data derived from oncomine (www.oncomine.org).

FIG. 39. CD44 mRNA level is up-regulated in human oral cavity carcinoma (left panel), head and neck squamous cell carcinoma (the middle panel), and tongue squamous cell carcinoma when compared to their normal counterparts. Data derived from oncomine (www.oncomine.org).

FIG. 40. hsCD44v3-v10-Fc, hsCD44v8-v10-Fc, and hsCD44s-Fc inhibits subcutaneous growth of human head and neck cancer cells in vivo. A, expression of CD44 by human head and neck carcinoma cells were assessed by Western blotting using anti-CD44 antibody (Santa Cruz). Expression level of CD44 by these carcinoma cells correlates with their tumorigenicity in vivo. B, overexpression of hsCD44s-Fc, hsCD44v8-v10-Fc, and hsCD44v3-v10-Fc fusion proteins by SCC-4 head-neck carcinoma cells. C, 5×10⁶ SCC-4 cells expressing different CD44-Fc fusion proteins or transduced with empty expression vectors were injected subcutaneously into each Rag-1 mice. Tumors were allowed to grow for ˜two months. At the end of experiments, all the tumors were dissected out and weighted. Data is presented as the mean of tumor weight+/−SD.

FIG. 41. A, Expression of CD44 by human pancreatic cancer cells (BXPC-3, PAN-08-13, PAN-08-27, and PAN-10-05) and a human hepatocellular carcinoma cell line (SK-Hep-1). B, overexpression of hsCD44s-Fc, hsCD44v8-v10-Fc, and hsCD44v3-v10-Fc fusion proteins by human BXPC-3 pancreatic cells (lane 1-3) or human SK-Hep-1 hepatocellular carcinoma cells (lane 4-6). C, 5×10⁶ BXPC-3 (C-a) or SH-Hep-1 (C-b) cells expressing different CD44-Fc fusion proteins or transduced with empty expression vectors were injected subcutaneously into each Rag-1 mice. Tumors were allowed to grow for ˜five-six weeks. At the end of experiments, all the tumors were dissected out and weighted. Data is presented as the mean of tumor weight (gram)+/−SD.

FIG. 42. CD44 mRNA is up-regulated in diffuse gastric adenocarcinoma (left panel), gastric mixed adenocarcinoma (middle panel), and gastric intestinal type adenocarcinoma (right panel) when compared to normal gastric mucosa. Data derived from oncomine (www.oncomine.org).

FIG. 43. CD44 mRNA is up-regulated in esophageal adenocarcinoma when compared to esophagus. Data derived from oncomine (www.oncomine.org)

FIG. 44. Higher levels of hyaluronan (HA) is detected in mouse plasma samples derived from the mice bearing breast cancer (MMTV-PyVT-634Mul/J and FVB-Tg-MMTV-Erbb2-NK1Mul/J mice) or implanted with gliomas (MSSM-GBMCSC-1 and G1261 cells) when compared to control mice that have no tumors. Plasma level of HA is measured using ELISA and biotinylated hsCD44-Fc fusion proteins.

FIG. 45. Western Blot of the expression of v-5 epitope tagged soluble CD44s. Cos-7 (lanes 1, 2, 3) and 293 cells (lanes 4, 5) were transduced with the retroviruses carrying empty expression vector (lanes 3) and the sCD44sv5 expression construct (lanes 1, 2, 4, 5). Puromycin-resistant pooled populations of Cos-7 and 293 cells were cultured for 48 hours and serum free cell culture supernatants were collected and analyzed by Western blots using anti-v5 mAb (Invitrogen).

FIG. 46. CD44 exon organization. 20 CD44 exons and 10 CD44 variant exons are shown. CD44s consists of exon 1-5, 16-18, and 20. TM stands for transmembrane and LCT stands for long cytoplasmic tail. Exon 19 encodes a short cytoplasmic tail. The NH₂-terminal common extracellular domain of CD44 (SEQ ID#7) is encoded by exon 1-5. Most of CD44 isoforms contain exon 1-5, exons 16-18, and exon 20 with or without different variant exons (v1-v10).

### DETAILED DESCRIPTION OF THE INVENTION

The present invention provides pharmaceutical compositions and methods for treating, preventing, or diagnosing cancers in a mammal. The present invention further provides pharmaceutical compositions and methods for treating or preventing gliomas in a mammal. The present invention provides pharmaceutical compositions and methods for treating or preventing of glioblastoma multiforme and other cancer types in a mammal. The present invention is further directed to pharmaceutical compositions and methods for sensitizing glioma cells and other types of cancer cells to oxidative, cytotoxic, and targeted therapeutic stresses for the treatment of gliomas and other cancer types. Oxidative stresses can be induced by, but not limited to, chemotherapy or radiation therapy. In one aspect, CD44 fusion proteins, acting as CD44 antagonists, are administered to a mammal for the treatment, prevention, or diagnosis of a glioma or other cancer types including colon cancer, breast cancer, prostate cancer, ovarian cancer, lung cancer, melanoma, renal cell carcinoma, gastric cancer, esophageal cancer, pancreatic cancer, liver cancer, and head-neck cancer. In another aspect, CD44 fusion proteins are administered alone and/or in combination with other therapeutic interventions to eliminate and/or suppress cancer stem cells. Targeted therapies can be inhibitors of EGFR, erbB-2, erbB-3, erbB-4 and c-Met receptor kinases or other receptor tyrosine kinases. In another aspect, targeted therapies are inhibitors of IAPs including cIAPs, XIAP, and survivin. In yet another aspect, targeted therapies are enhancers/stimulators/stabilizer of p53, p21, puma, and p38/JNK kinases. In another aspect, targeted therapies are the agents that promote or induce apoptotic stresses to cancer cells including inhibitors of PI3K, mTOR, proteasome inhibitor, and angiogenesis inhibitors. In yet another aspect, targeted therapies are inhibitors of Wnt signaling pathway.

### Gliomas

Gliomas are the most common type of primary brain cancer and constitute a spectrum of tumors of variable degrees of differentiation and malignancy that may arise from the transformation of neural progenitor cells (Giese et al., 2003; Maher et al., 2001). The most aggressive of these tumors is grade IV astrocytoma, also known as glioblastoma multiforme (GBM), that by virtue of its resistance to chemotherapy, radiotherapy, and established targeted therapies, is incurable (Davis et al., 1998). As demonstrated in the present Examples, gliomas express elevated levels of a major cell surface HA receptor, CD44.

Resistance to cytotoxic agents, radiation, and targeted therapies constitutes the major obstacle to successful treatment of GBM and other malignant cancers. Increasing evidence suggests the existence of cancer stem cells (CSC), including glioma CSCs, that are highly resistant to chemo- and radiation therapy and are likely to be responsible for the recurrence of malignant cancer, including GBM, following therapeutic intervention (Hambardzumyan et al., 2008; Reya et al., 2001). Although the implication of CD44 in the formation and maintenance glioblastoma CSC is just started to be uncovered, CD44 has been established as a major cell surface CSC marker in numerous tumors including leukemia and cancers of the breast, colon, ovary, prostate, pancreas, and head-neck (Croker and Allan, 2008; Reya et al. 2001; Stamenkovic and Yu, 2009). CD44 has been shown to be required for engraftment of leukemia CSC in the bone marrow (Jin et al., 2006; Krause et al., 2006) and to be functionally relevant for colorectal cancer CSC (Du et al., 2008). These observations suggest a potentially important role of CD44 in CSC maintenance and/or function. The present Examples demonstrate that CD44 attenuates the activation of the Hippo stress/apoptotic signaling pathway in GBM cells and protects GBM cells from temozolomide (TMZ) and oxidative stress in vitro and provides a chemoprotective function in vivo. Furthermore, knockdown of CD44 expression inhibits self-renewal capacity of glioma spheres and expression of CD44 antagonism, hsCD44s-Fc fusion protein inhibits in vivo growth of GBMCSCs, suggesting an important role of CD44 in cancer stem cell maintenance.

### Breast Cancer

Breast cancer is the most common cancer among women in the United States and the second leading cause of cancer related death in women. Due to improved early detection and treatment, breast cancer death rates are going down. However, there are still estimated 40,170 breast cancer related deaths in year 2009 (http://www.cancer.gov/cancertopics/types/breast), which is largely caused by the abilities of breast cancer cells to metastasize and develop resistance to current therapies. This reality urgently begs for more effective and targeted novel therapies that battle these deadly abilities of malignant breast cancer. Recent advances in cancer stem cell (CSC) field have indicated that therapeutic resistance and recurrence of malignant cancers including breast cancer are likely due to existence of a small subset of CSCs including breast CSCs (BCSCs) that are highly resistant to therapeutic interventions (Al-Hajj et al., 2003; Dean et al., 2005; Reya et al., 2001). CSCs are characterized by their ability to self-renew, differentiate into various lineages, and reconstitute the cellular hierarchy of the tumor (Al-Hajj et al., 2003; Reya et al., 2001).

Breast cancers consist of heterogeneous cell populations including tumor cells and host stroma. Much of cancer research has been focus on cancer cells. Increasing evidence has indicated that the host micro-environment plays essential roles in breast cancer progression and regulating their response to therapies (Al-Hajj et al., 2003; Liu et al., 2007). Furthermore, maintenance of BCSCs requires adequate host microenvironment niche. Therefore, it is essential to develop new therapeutic agents that target BCSCs and their microenvironment niche in order to eradicate this deadly disease. Physical interactions and functional cross-talk between tumor cells and their micro-environment are mediated primarily by cell surface receptors that are responsible for the cell-cell and cell-ECM (extracellular matrix) adhesion. CD44 is a major cell surface receptor for hyaluronan (HA), an abundant component of ECM, as well as a key marker for CSCs including BCSCs (Collins et al., 2005; Patrawala et al., 2006; Ponti et al., 2005; Reya et al., 2001). CD44+/CD24− BCSCs display increased tumorigenicity, metastatic potential, and chemoresistance (Collins et al., 2005; Reim et al., 2009; Shipitsin et al., 2007). Accumulation of the CD44 ligand, HA, in breast cancer stroma is correlated with an unfavorable prognosis (Tammi et al., 2008).

### Prostate Cancer

Prostate cancer is the second leading cause of cancer-related death in American men. Prognosis for hormone-independent/refractory metastatic prostate cancer (HRPC) is very poor and treatment options for the late stage disease are limited. Therefore, there is an urgent need to develop more effective and targeted novel therapies to combat this deadly disease. To achieve that, it is essential to first identify novel targets that play key roles in prostate cancer progression, metastasis, and resistance to chemotherapy. Recent advances in CSC research demonstrated that CSCs are highly resistant to chemo- and radio-therapy and are believed to be responsible for tumor recurrence following therapeutic intervention (Dean et al., 2005; Reya et al., 2003). CD44 is a predominant cell surface marker for a variety of human cancer stem or initiating cells including that of prostate cancers (Collins et al., 2005; Hurt et al., 2008; Maitland and Collins, 2008).

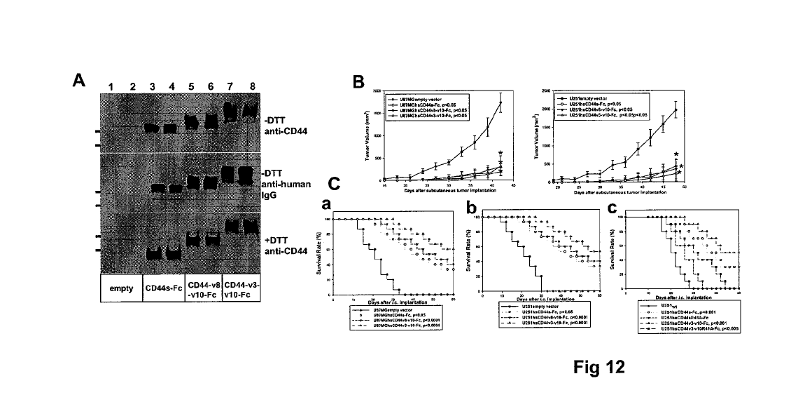

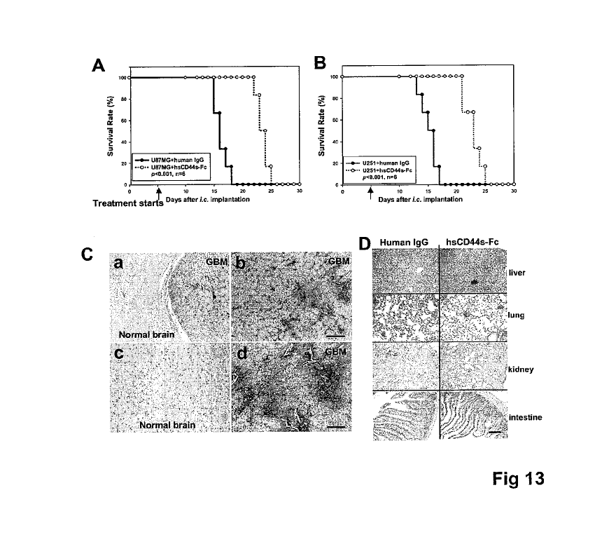

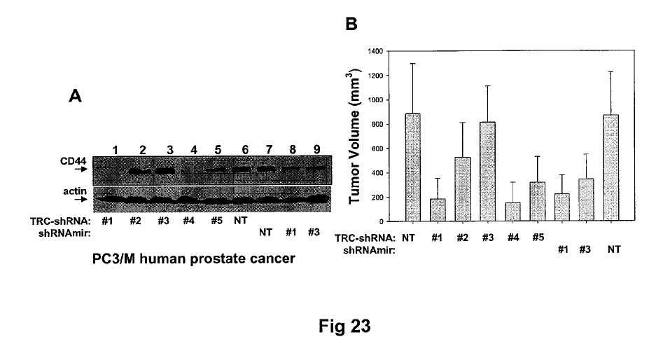

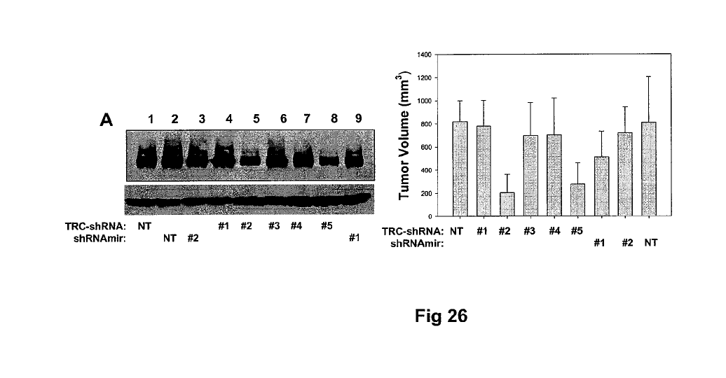

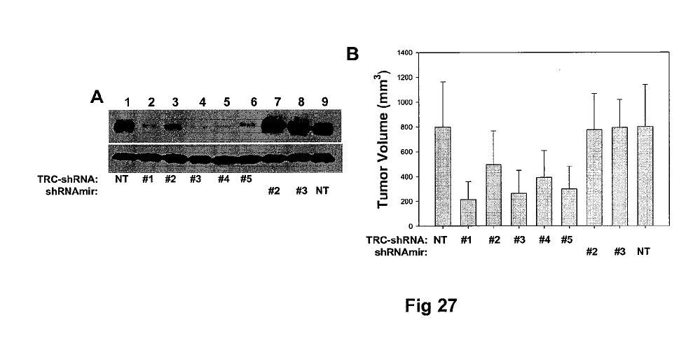

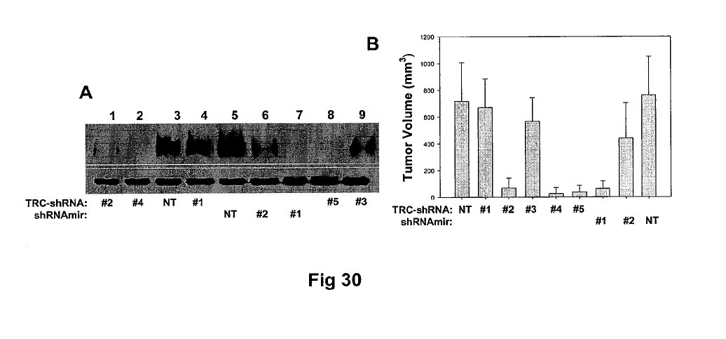

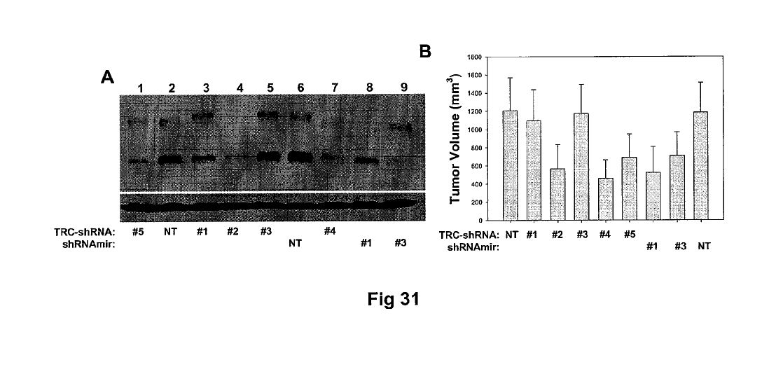

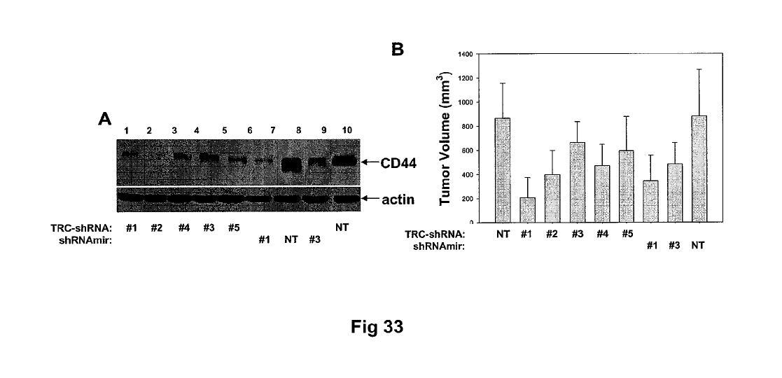

Current anti-cancer therapeutic strategies and target selection are heavily concentrated on frequently mutated kinases whose activity cancer cells appear to become addicted (Sharma et al., 2007). Although these approaches are conceptually sound and supported by notable successes, they are hampered by the emergence of resistant tumor cells capable of bypassing the targeted signaling pathways through mechanisms that may be related to the mutated nature of the target itself. It is now well accepted that therapeutic interventions targeting only a single signaling pathway, no matter how seemingly important, are relatively easily evaded by cancer cells as they acquire new genetic and epigenetic alterations. An alternative strategy may therefore be to identify versatile molecules that, unlike the key drivers of oncogenesis, are not central to any single functional tumor cell property but participate in multiple functions, including the modulation of diverse signaling pathways as co-receptors, interactions between tumor cells and the host tissue microenvironment, and responses of tumor cells to various forms of stresses. Based on their obvious usefulness for tumor growth and progression, such molecules are likely to be upregulated in malignant tumors but unlikely to be frequently mutated. Selective inhibition of these types of broad-spectrum targets that play essential roles in mediating tumor-host interaction and in modulating activities of several important signaling pathways, especially in combination with chemo- and radiation therapy, and targeted therapies against these essential signaling pathways and/or promote/induce stresses to cancer cells, may therefore overcome the drug resistance obstacle of current cancer treatment and achieve more efficacious and/or longer lasting clinical benefits. The present invention indicates that CD44 is one such target for multiple cancers, and antagonists of CD44, which includes soluble human CD44 fusion proteins such as CD44-Fc fusion proteins, are effective anti-cancer agents. Our preclinical results provide strong support for the therapeutic potential of targeting CD44 in malignant glioma, breast cancer, prostate cancer, melanoma, pancreatic cancer, liver cancer, and head-neck cancer, colon cancer, ovarian cancer, and lung cancer. For example, FIGS. 3-4, 23, 26-27, 30-31, AND 33 show that shRNAs against human CD44 inhibit in vivo growth of human glioblastoma (Xu et al., 2010), colon cancer, breast cancer, prostate cancer, lung cancer, and ovarian cancer in animal models. FIGS. 12, 17, 36, 40, and 41 show that expression CD44-Fc fusion proteins inhibits in vivo growth of human glioblastoma, melanoma, head-neck carcinoma, pancreatic cancer, and liver cancer in animal models. FIGS. 13, 24, and 29 show that purified CD44-Fc fusion proteins inhibits in vivo growth of human glioblastoma, breast cancer, and prostate cancer in animal models. Together, these comprehensive data establish that CD44 is a prime therapy target in these cancer types and that CD44 antagonists, including CD44 fusion proteins and shRNAs against CD44, are effective agents against human glioblastoma, colon cancer, breast cancer, prostate cancer, lung cancer, ovarian cancer, melanoma, head-neck carcinoma, pancreatic cancer, and liver cancer.

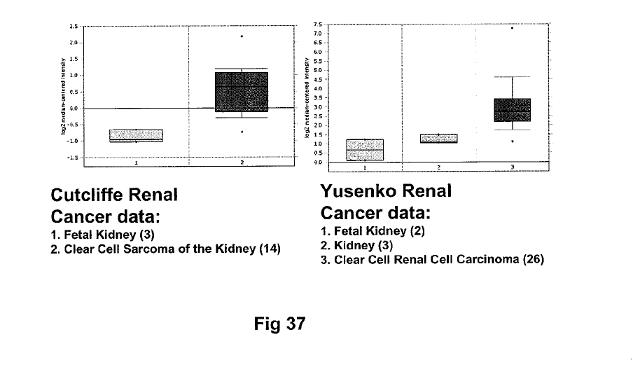

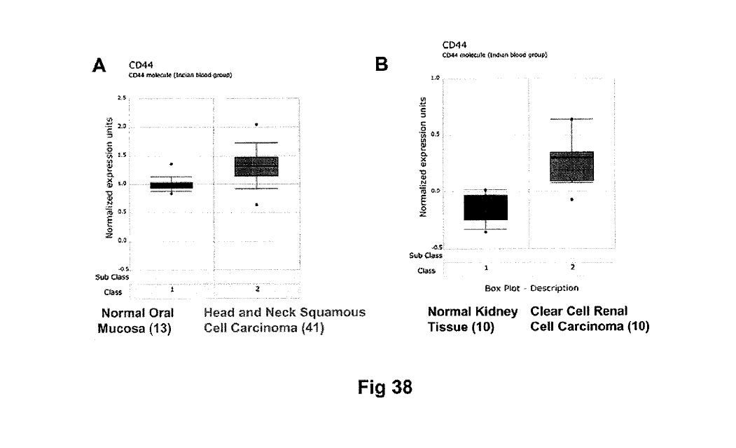

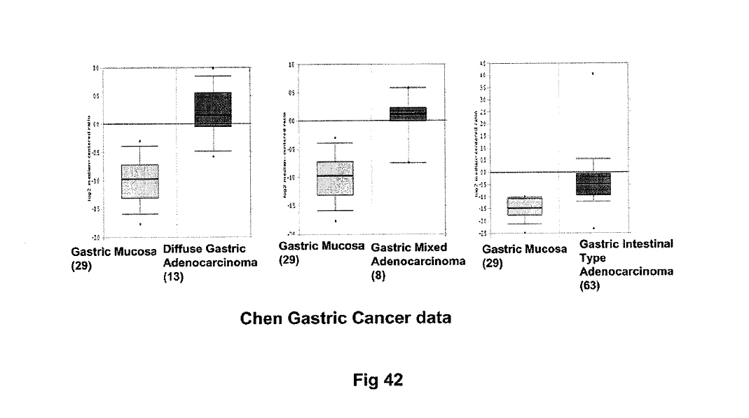

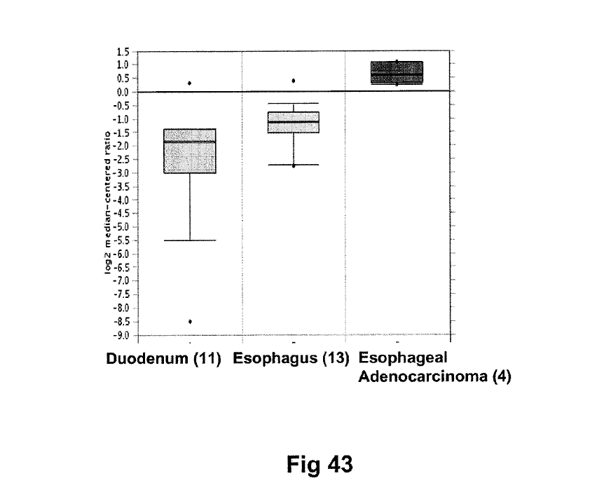

Based on the fact that CD44 is up-regulated and/or plays an important role in various cancer types, CD44 may serve as therapeutic target for the following cancers in addition to human gliomas, colon cancer, breast cancer, prostate cancer, lung cancer, ovarian cancer, melanoma (Stamenkovic and Yu, 2009), head-neck carcinoma (Aillles and Prince, 2009; Nelson and Grandis, 2007), pancreatic cancer (Hong et al., 2009; Klingbeil et al., 2009; Lee et al., 2008), and liver cancer (Barbour et al., 2003; Yang et al., 2008), malignant mesothelioma (Ramos-Nino et al., 2007; Tajima et al., 2010), sarcomas (Yoshida et al., 2008), renal-cell carcinoma (FIG. 37-38,) (Lim et al., 2008; Lucin et al., 2004; Yildiz et al., 2004), cancer of the esophagus (FIG. 43) (Li et al., 2005; Nozoe et al., 2004), Wilms' tumor (Ghanem et al., 2002), bladder carcinoma (Stavropoulos et al., 2001), multiple myeloma (Mitsiades, 2005; Ohwada et al., 2008), Gastric Cancer (FIG. 42), and schwannomas (Bai et al., 2007).

### CD44 and Cell Signaling

CD44 has been implicated in the modulation of several signaling pathways. It serves as a co-receptor of c-Met (Matzke et al., 2007) and modulates signals from the ErbB family of RTKs (Turley et al., 2002). CD44 activates c-Src and focal adhesion kinase (FAK) (Turley et al., 2002) and promotes cell motility through activation of Rac1 (Murai et al., 2004). However, no single core intact pathway that mediates CD44 derived signal has been established thus far. CD44 interacts with the ERM family proteins (Tsukita and Yonemura, 1997) and merlin (Morrison et al., 2001; Sainio et al., 1997), the product of the neurofibromatosis type 2 (NF2) gene. Merlin mutations or loss of merlin expression cause NF2 disease, characterized by the development of schwannomas, meningiomas, and ependymomas (Gutmann et al., 1997; Kluwe et al., 1996). In Drosophila, merlin functions upstream of the Hippo signaling pathway, but a definitive link between merlin and the mammalian Hippo pathway orthologs has not been fully established. We have shown recently that merlin is a potent inhibitor of human GBM growth and that it functions upstream of MST1/2 by activating MST1/2-Lats2 signaling in glioma cells (Lau et al., 2008). These observations suggest that the mammalian Hippo signaling pathway may play an important role in GBM progression. The present invention demonstrates that cancer cells with depleted endogenous CD44 responded to oxidative and cytotoxic stresses with robust and sustained phosphorylation/activation of MST1/2 and Lats1/2, phosphorylation/inactivation of YAP, and reduced expression of cIAP1/2. These effects correlate with reduced phosphorylation/inactivation of merlin and increased levels of cleaved caspase-3. By contrast, a higher level of endogenous CD44 promotes phosphorylation/inactivation of merlin, inhibits the stress induced activation of the mammalian equivalent of Hippo signaling pathway, and up-regulates cIAP1/2, leading to the inhibition of caspase-3 cleavage which is an indicator of apoptosis. Together, these results place CD44 upstream of the mammalian Hippo signaling pathway (merlin-MST1/2-Lats1/2-YAP-cIAP1/2) and suggest a functional role for CD44 in attenuating tumor cell responses to stress and stress-induced apoptosis.

Furthermore, the present invention demonstrates that knockdown of CD44 results in elevated and sustained activation of p38/JNK stress kinases, known effectors of MST1/2 kinases, in glioma cells exposed to oxidative and cytotoxic stress. In addition, oxidative stress induced a sustained up-regulation of p53, a known downstream effector of JNK/p38, and its target genes p21 and puma in CD44-deficient glioma cells, whereas the GBM cells with high levels of endogenous CD44 attenuated activation of JNK/p38, and inhibited induction of p53, p21, and puma. These mechanistic results suggest that CD44 antagonists, including CD44 fusion proteins, can be used in synergy with pharmacological enhancers/stimulators/stabilizers of p53, p21, puma, and p38/JNK kinases and with inhibitors of IAPs, including cIAPs and XIAP, to achieve a better clinical outcome.

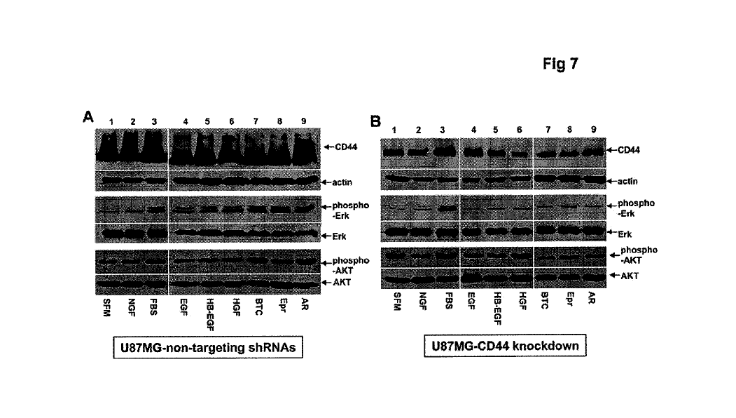

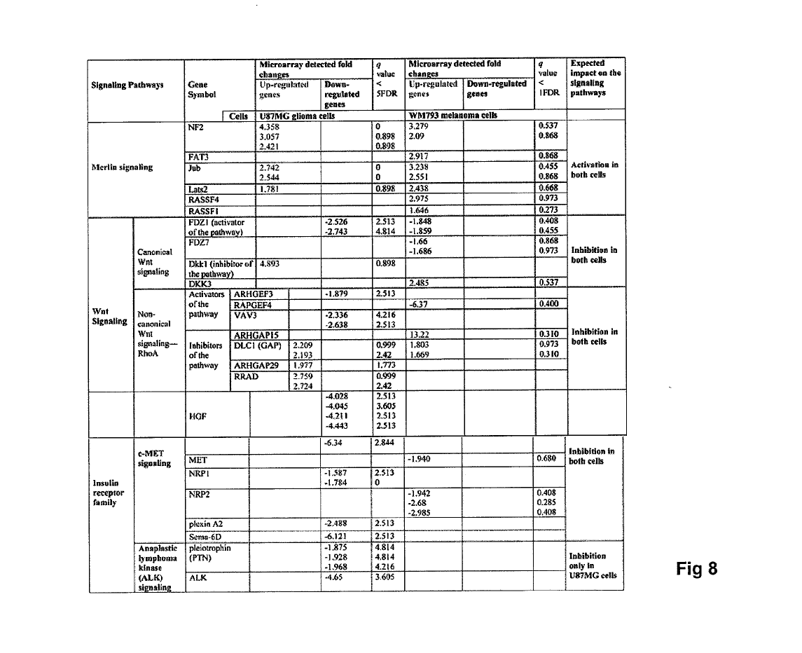

Receptor tyrosine kinases (RTKs) play a central role in a variety of normal cellular functions, transformation, and tumor progression. Hepatocyte growth factor (HGF) and its receptor c-Met are known to promote brain tumor growth and progression (Abounader and Laterra, 2005). Increased expression of HGF and c-Met frequently correlates with glioma grade, blood vessel density, and poor prognosis. Moreover, over expression of HGF and/or c-Met enhances whereas their inhibition blocks gliomagenesis (Abounader and Laterra, 2005). In addition, amplification of the EGFR gene occurs in approximately 40% of GBM cases and constitutes as a predictor of poor prognosis (Voelzke et al., 2008). The present invention demonstrates that depletion of CD44 inhibits Erk1/2 activation induced by EGFR ligands and HGF but not by NGF or fetal bovine serum (FBS; FIG. 7), suggesting that CD44 serves as a co-receptor/stimulator for these RTKs and enhances their signaling activity in malignant glioma cells and other cancer types. Although the precise mechanism whereby CD44 regulates RTK signaling requires further investigation, its function as an HA receptor provides a possible explanation. CD44 forms large aggregates on the cell surface upon engagement by its multivalent ligand, HA. These aggregates often reside in lipid rafts or other specialized membrane domains where initiation of multiple signaling events occurs. In addition, CD44 can be expressed as a cell surface proteoglycan that binds numerous heparin binding growth factors including HB-EGF and basic FGF. As an RTK co-receptor, CD44 can therefore enhance signaling by facilitating RTK oligomerization and/or presenting the appropriate ligands to the corresponding RTKs. These mechanistic results suggest that CD44 antagonists, including CD44 fusion proteins, can be used in synergy with the pharmacological inhibitors of EGFR, erbB-2, erbB-4 and c-Met receptor kinases to achieve a better clinical outcome.

### CD44 Fusion Proteins

Because CD44 is a receptor for multiple ligands, the strategy of using fusion proteins of the extracellular domain of CD44 with non-CD44 molecules, such as the constant region of human IgG1 (Fc), is superior to the functional blocking antibodies against CD44. Each blocking antibody of CD44 can only block the interaction of one or a few ligands, whereas CD44-Fc fusion proteins block all the interaction between CD44 and its ligands mediated by the extracellular domain of CD44. In addition, CD44 is shed from the cell surface by proteases, which is thought to be a functionally important process that triggers signaling pathways and regulates CD44-mediated functions (Stamenkovic and Yu, 2009). Soluble CD44 fusion proteins contain the domain that interacts with CD44 sheddase(s); therefore CD44 fusion proteins are capable of blocking shedding as well as sequestering all the CD44 ligands. These characteristics of CD44 fusion proteins provide advantages for antagonizing CD44 function.

CD44-Fc proteins, which are fusion proteins between the different segments of the extracellular domain of CD44 with the constant region of human IgG1 (Fc), act as “trap” type fusion proteins of a multifunctional transmembrane receptor, which not only target bulk of tumors, cancer stem cells, but also tumor microenvironment (such as infiltrating host cells including but not limited to endothelial cells, pericytes, leukocytes, inflammatory cells, and fibroblasts, tumor-host cell interaction, and tumor-host ECM interaction). Key interactions and cross-talk between tumor cells and their microenvironment are mediated by surface receptors including cell-cell adhesion and ECM receptors, which provide potentially attractive therapeutic targets (Marastoni et al., 2008). The expression of CD44 is often higher in tumor cells, cancer stem cells, and tumor microenvironment, but lower in normal tissues; therefore, CD44 serves as an ideal target for cancer therapy.

CD44 protein consists of an extracellular domain with an NH₂-terminal HA-binding region and a membrane-proximal region, a transmembrane domain (TM), and a COOH-terminal cytoplasmic tail (CT) (Peach et al., 1993; Stamenkovic et al., 1989). There is a single CD44 gene containing 20 exons. At least 10 of these exons, exons 6-15 or variant exons v1-v10, can be alternatively spliced to give rise to numerous CD44 variants (Screaton et al., 1993; Screaton et al., 1992). The standard form of CD44 (CD44s) is a product of alternative splicing of transcript and consists of all the common exons 1-5, 16-18 and 20.

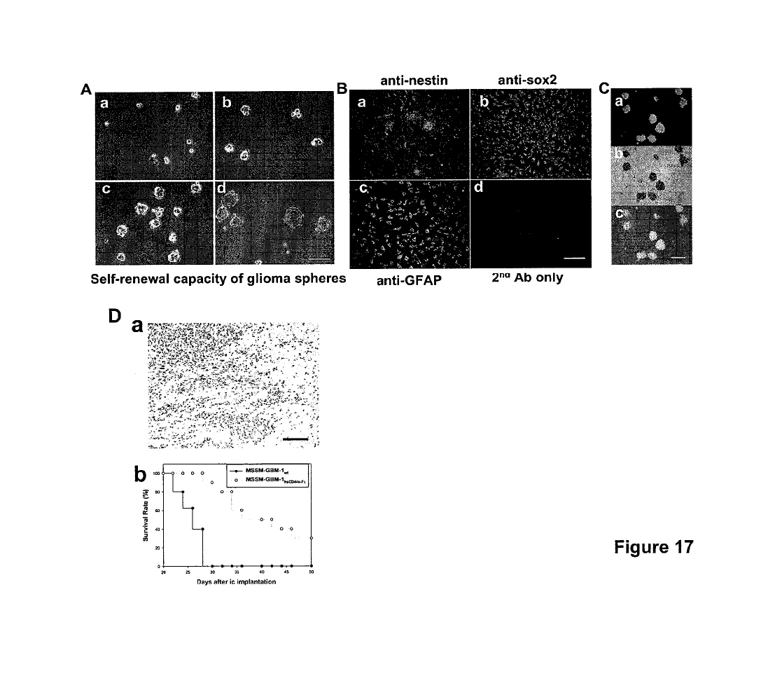

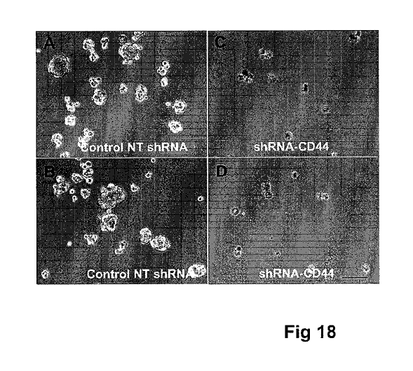

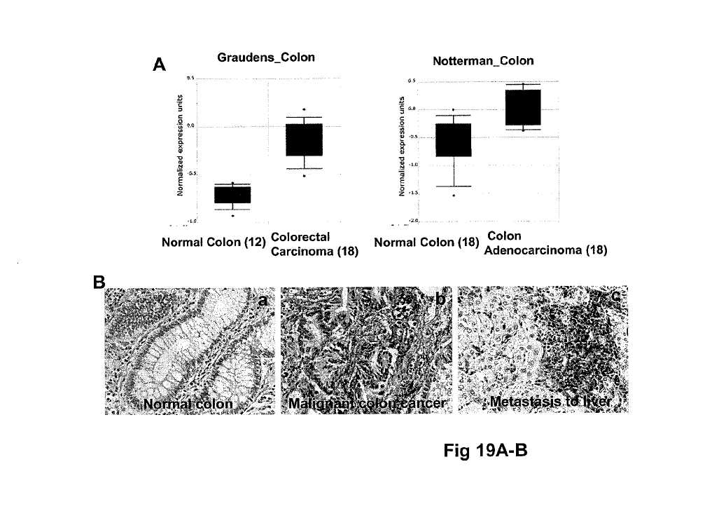

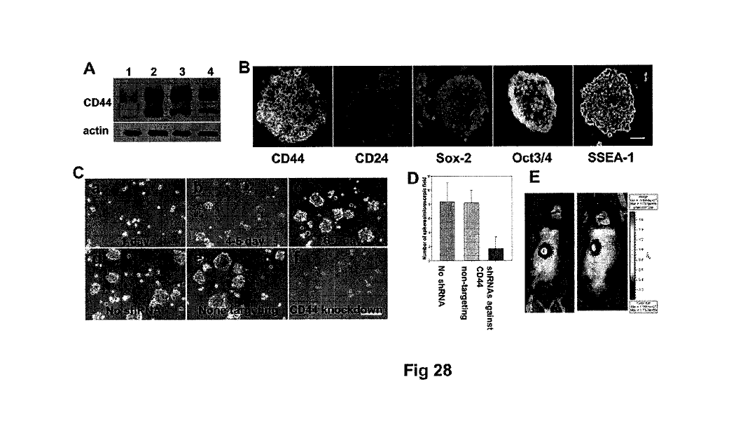

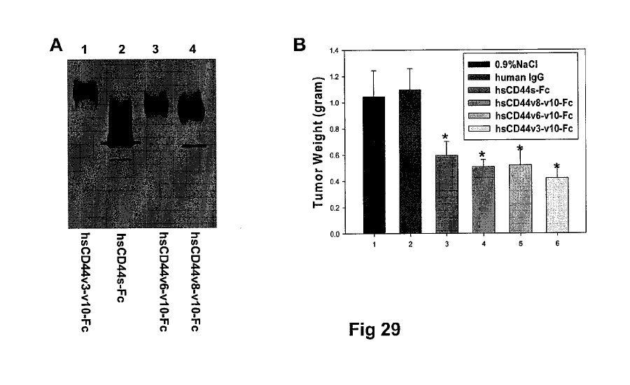

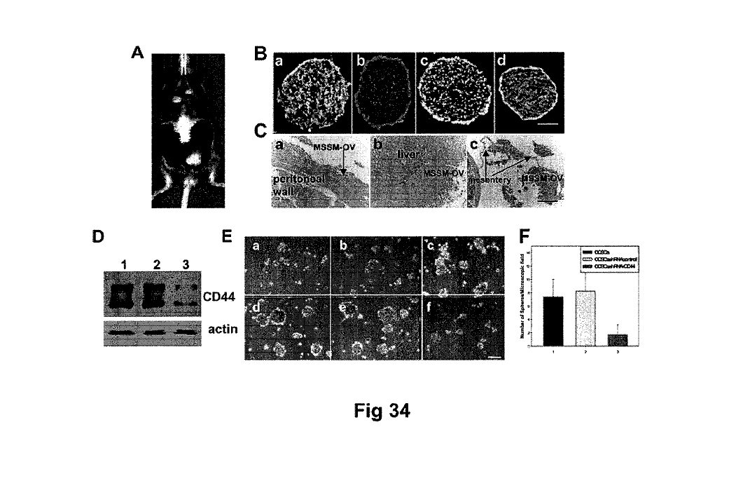

In one embodiment of the present invention, CD44 fusion proteins, acting as CD44 antagonists, are administered to a mammal for the treatment or prevention of a glioma or other cancer types. In one aspect, CD44 fusion proteins are administered prior to, simultaneously with, or after an additional anti-cancer therapy or surgical removal of tumors including glioma. CD44 is important for cancer stem cells as we have shown that CD44 depletion inhibits the formation of glioma spheres (FIG. 18), mammospheres (FIG. 28C), and ovarian CSC spheres (FIG. 34E) and that overexpression of CD44 inhibits GBMCSC growth in vivo (FIG. 17D) and purified CD44-Fc fusion proteins inhibited growth of BCSCs in vivo (FIG. 29). Because CSCs often survive cancer therapies, treatment with CD44 fusion proteins following these therapies is important to prevent the recurrence and metastasis of cancer, even in the absence of visible disease. In another aspect, the CD44 fusion proteins are administered prior to, simultaneously with, or after a treatment which causes cytotoxic stresses or other forms of stresses. In yet another aspect, CD44 fusion proteins are administered as single agents or along with other anti-cancer therapies prior to or after surgery to treat glioma and other cancer types, wherein the administration of CD44 fusion proteins and the additional therapeutic agents provides a synergistic effect. In one aspect, CD44 fusion protein is administrated as purified protein. In another aspect, CD44 fusion protein is administrated in the form of viral expression vector with or without being packaged into viral particles or using nanoparticles as carriers. In one aspect, the viral particle is retrovirus, lentivirus, adenovirus, or adeno-associated virus (AAV). In one aspect, the adenovirus is a replication-impaired, non-integrating, serotype 2, 5, 6, 7, or 8 adenoviral vector. In one aspect, the CD44 fusion protein is a CD44-Fc fusion protein, which comprises the constant region of human IgG1 fused to different segments of the extracellular domain of CD44. In another aspect, the CD44 extracellular domain is derived from CD44s, CD44v3-v10, CD44v6-v10, CD44v8-v10, CD44sR41A, CD44v3-v10R41A, CD44v6-v10R41A, and CD44v8-v10R41A. In yet another aspect, the CD44 extracellular domain is derived from CD44v4-v10, CD44v5-v10, CD44v7-v10, CD44v9-v10, CD44v3, CD44v4, CD44v5, CD44v6, CD44v7, CD44v9, CD44v10, or the above CD44 isoforms containing the R41A mutation. In yet another aspect, the CD44 extracellular domain is derived from different combinations of exons 1-17, different deletions, mutations, duplication, or multiplication of the different segments of the extracellular domain of CD44.

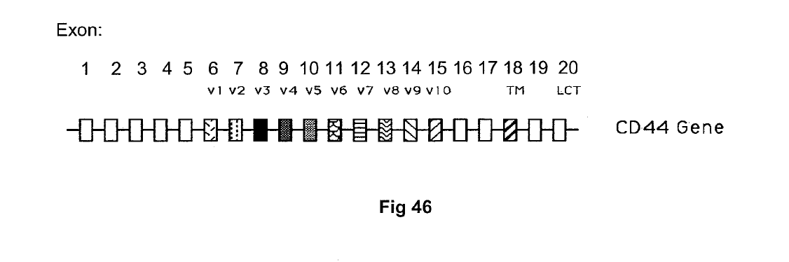

The extracellular domain of CD44 is encoded by exon 1-5, v1-v10 (or exon 6-10), exon 16, and exon 17. The extracellular domain of CD44s consists of exon 1-5, 16, and 17. CD44 variants (CD44v2-v10, CD44v3-v10, CD44v8-v10, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, and CD44v2) consist of exon 1-5, different combinations of the variant exons (v2-v10), exon 16, and exon 17 (FIG. 46).

TABLE 1
Nucleotide and Amino Acid Sequences for the constant region (Fc) of Homo sapien
Immunoglobulin Heavy Constant Gamma 1, Human CD44 Wild Type Extracellular
Domains, Human CD44 Extracellular Domains containing R41A mutation, and CD44-Fc
Fusion Proteins

| | | SEQ |
|---|---|---|
| | | ID |
| Protein | Sequence | No. |
| Constant | gacaaaactc | 1 |
| region | acacatgccc accgtgccca gcacctgaac tcctgggggg accgtcagtc ttectettcc | |
| (Fc) of Homo | ccccaaaacc caaggacacc ctcatgatct cccggacccc tgaggtcaca tgcgtggtgg | |
| sapien | tggacgtgag ccacgaagac cctgaggtca agttcaactg gtacgtggac ggcgtggagg | |
| immuno- | tgcataatgc caagacaaag ccgcgggagg agcagtacaa cagcacgtac cgtgtggtca | |
| globulin | gcgtcctcac cgtcctgcac caggactggc tgaatggcaa ggagtacaag tgcaaggtct | |
| heavy | ccaacaaagc cctcccagcc cccatcgaga aaaccatctc caaagccaaa gggcagcccc | |
| constant | gagaaccaca ggtgtacacc ctgcccccat cccgggatga gctgaccaag aaccaggtca | |
| gamma 1 | gcctgacctg cctggtcaaa ggcttctatc ccagcgacat cgccgtggag tgggagagca | |
| | atgggcagcc ggagaacaac tacaagacca cgcctcccgt gctggactcc gacggctcct | |
| | tcttcctcta cagcaagctc accgtggaca agagcaggtg gcagcagggg aacgtcttct | |
| | catgctccgt gatgcatgag gctctgcaca accactacac gcagaagagc ctctccctgt | |
| | ctccgggtaa atga | |
| | DKTHTCPP CPAPELLGGP SVFLFPPKPK DTLMISRTPE VTCVVVDVSH | 41 |
| | EDPEVKFNWY VDGVEVHNAK TKPREEQYNS TYRVVSVLTV | |
| | LHQDWLNGKE YKCKVSNKAL | |
| | PAPIEKTISK AKGQPREPQV YTLPPSRDEL TKNQVSLTCL VKGFYPSDIA | |
| | VEWESNGQPE | |
| | NNYKTTPPVL DSDGSFFLYS KLTVDKSRWQ QGNVFSCSVM | |
| | HEALHNHYTQ KSLSLSPGK | |
| Human CD44 | atggac aagttttggt ggcacgcagc ctggggactc tgcctcgtgc cgctgagcct ggcg | 2 |
| signal peptide | MDKFWWHAAWGLCLVPLSLA | 42 |
| Constant | atggac aagttttggt ggcacgcagc ctggggactc tgcctcgtgc cgctgagcct ggcg gacaaaactc | 3 |
| region(Fc) of | acacatgccc accgtgccca gcacctgaac tcctgggggg accgtcagtc ttcctcttcc | |
| immunoglobulin | ccccaaaacc caaggacacc ctcatgatct cccggacccc tgaggtcaca tgcgtggtgg | |
| heavy constant | tggacgtgag ccacgaagac cctgaggtca agttcaactg gtacgtggac ggcgtggagg | |
| gamma 1 with | tgcataatgc caagacaaag ccgcgggagg agcagtacaa cagcacgtac cgtgtggtca | |
| CD44 signal | gcgtcctcac cgtcctgcac caggactggc tgaatggcaa ggagtacaag tgcaaggtct | |
| peptide | ccaacaaagc cctcccagcc cccatcgaga aaaccatctc caaagccaaa gggcagcccc | |
| | gagaaccaca ggtgtacacc ctgcccccat cccgggatga gctgaccaag aaccaggtca | |
| | gcctgacctg cctggtcaaa ggcttctatc ccagcgacat cgccgtggag tgggagagca | |
| | atgggcagcc ggagaacaac tacaagacca cgcctcccgt gctggactcc gacggctcct | |
| | tcttcctcta cagcaagctc accgtggaca agagcaggtg gcagcagggg aacgtcttct | |
| | catgctccgt gatgcatgag gctctgcaca accactacac gcagaagagc ctctccctgt | |
| | ctccgggtaa atga | |
| | MDKFWWHAAWGLCLVPLSLA DKTHTCPP CPAPELLGGP SVFLFPPKPK | 43 |
| | DTLMISRTPE VTCVVVDVSH | |
| | EDPEVKFNWY VDGVEVHNAK TKPREEQYNS TYRVVSVLTV | |
| | LHQDWLNGKE YKCKVSNKAL | |
| | PAPIEKTISK AKGQPREPQV YTLPPSRDEL TKNQVSLTCL VKGFYPSDIA | |
| | VEWESNGQPE | |
| | NNYKTTPPVL DSDGSFFLYS KLTVDKSRWQ QGNVFSCSVM | |
| | HEALHNHYTQ KSLSLSPGK | |
| Wild type | atggac aagttttggt ggcacgcagc ctggggactc tgcctcgtgc | 4 |
| extracellular | cgctgagcct ggcg cagatc gatttgaata taacctgccg ctttgcaggt gtattccacg | |
| domain of | tggagaaaaa tggtcgctac agcatctctc ggacggaggc cgctgacctc tgcaaggctt | |
| CD44s | tcaatagcac cttgcccaca atggcccaga tggagaaagc tctgagcatc ggatttgaga | |
| (exon 1-5, 16 | cctgcaggta tgggttcata gaagggcacg tggtgattcc ccggatccac cccaactcca | |
| and17) | tctgtgcagc aaacaacaca g,gggtgtaca tcctcacatc caacacctcc cagtatgaca | |
| | catattgctt caatgcttca gctccacctg aagaagattg tacatcagtc acagacctgc | |
| | ccaatgcctt tgatggacca attaccataa ctattgttaa ccgtgatggc acccgctatg | |
| | tccagaaagg agaatacaga acgaatcctg aagacatcta ccccagcaac cctactgatg | |
| | atgacgtgag cagcggctcc tccagtgaaa ggagcagcac ncaggaggt tacatctta | |
| | acaccattc tactgtacac cccatcccag acgaagacag tccctggatc accgacagca | |
| | cagacagaat ccctgctacc aga gac caagacacat tccaccccag tggggggtcc cataccactc | |
| | atggatctga atcagatgga cactcacatg ggagtcaaga aggtggagca aacacaacct ctggtcctat | |
| | aaggacaccc caaattccag aa | |
| | MDKFWWHAAWGLCLVPLSLAQIDLNITCRFAGVFHVEKNGRYSI | 44 |
| | SRTEAADLCKAFNSTLPTMAQMEKALSIGFETCRYGFIEGHVVIPRIHPNSI | |
| | CAANNT | |
| | GVYILTSNTSQYDTYCFNASAPPEEDCTSVTDLPNAFDGPITITIVNRDGTRYVQK | |
| | GE | |
| | YRTNPEDIYPSNPTDDDVSSGSSSERSSTSGGYIFYTFSTVHPIPDEDSPWITDSTD | |
| | R IPAT R DQDTF HPSGGSHTTHGSESDGHSHGSQEGGANTTSGPIRTPQIPE | |
| extracellular | atggac aagttttggt ggcacgcagc ctggggactc tgcctcgtgc | 5 |
| domain of CD44s | cgctgagcct ggcg cagatc gatttgaata taacctgccg ctttgcaggt gtattccacg | |
| with a R41A | tggagaaaaa t ggt gcc tac agc atc tctc ggacggaggc cgctgacctc tgcaaggctt | |
| mutation (exon | tcaatagcac cttgcccaca atggcccaga tggagaaagc tctgagcatc ggatttgaga | |
| 1-5, 16, and 17) | cctgcaggta tgggttcata gaagggcacg tggtgattcc ccggatccac cccaactcca | |
| | tctgtgcagc aaacaacaca ggggtgtaca tcctcacatc caacacctcc cagtatgaca | |
| | catattgctt caatgcttca gctccacctg aagaagattg tacatcagtc acagacctgc | |
| | ccaatgcctt tgatggacca attaccataa ctattgttaa ccgtgatggc acccgetatg | |
| | tccagaaagg agaatacaga acgaatcctg aagacatcta ccccagcaac cctactgatg | |
| | atgacgtgag cagcggctcc tccagtgaaa ggagcagcac ttcaggaggt tacatctttt | |
| | acaccttttc tactgtacac cccatcccag acgaagacag tccctggatc accgacagca | |
| | cagacagaat ccctgctacc aga gac caagacacat tccaccccag tggggggtcc cataccactc | |
| | atggatctga atcagatgga cactcacatg ggagtcaaga aggtggagca aacacaacet | |
| | ctggtcetat aaggacaccc caaattccag aa | |
| | MDKFWWHAAWGLCLVPLSLAQIDLNITCRFAGVFHVEKNGAYSI | 45 |
| | SRTEAADLCKAENSTLPTMAQMEKALSIGFETCRYGFIEGHVVIPRIHPNSI | |
| | CAANNT | |
| | GVYILTSNTSQYDTYCFNASAPPEEDCTSVTDLPNAFDGPITITIVNRDGTRYVQK | |
| | GE | |
| | YRTNPEDIYPSNPTDDDVSSGSSSERSSTSGGYIFYTFSTVHPIPDEDSPWITDSTD | |
| | R IPAT R DQDTF HPSGGSHTTHGSESDGHSHGSQEGGANTTSGPIRTPQIPE | |
| CD44 NH₂- | atggac aagttttggt ggcacgcagc ctggggactc tgcctcgtgc | 6 |
| terminal | cgctgagcct ggcg cagatc gatttgaata taacctgccg ctttgcaggt gtattccacg | |
| common | tggagaaaaa t ggt gcc tac agc atc tct c gg acggaggc cgctgacctc tgcaaggctt | |
| extracellular | tcaatagcac cttgcccaca atggcccaga tggagaaagc tctgagcatc ggatttgaga | |
| domain with a | cctgcaggta tgggttcata gaagggcacg tggtgattcc ccggatccac cccaactcca | |
| R41A mutation | tctgtgcagc aaacaacaca ggggtgtaca tcctcacatc caacacctcc cagtatgaca | |
| (exon 1-5) | catattgctt caatgcttca gctccacctg aagaagattg tacatcagtc acagacctgc | |
| | ccaatgcctt tgatggacca attaccataa ctattgttaa ccgtgatggc acccgctatg | |
| | tccagaaagg agaatacaga acgaatcctg aagacatcta ccccagcaac cctactgatg | |
| | atgacgtgag cagcggctcc tccagtgaaa ggagcagcac ttcaggaggt tacatctttt | |
| | acaccttttc tactgtacac cccatcccag acgaagacag tccctggatc accgacagca | |
| | cagacagaat ccctgctacc agt | |
| | MDKFWWHAAWGLCLVPLSLAQIDLNITCRFAGVFHVEKNGAYSI | 46 |
| | SRTEAADLCKAFNSTLPTMAQMEKALSIGFETCRYGFIEGHVVIPRIHPNSICAAN | |
| | NT | |
| | GVYILTSNTSQYDTYCFNASAPPEEDCTSVTDLPNAFDGPITITIVNRDGTRYVQK | |
| | GE | |
| | YRTNPEDIYPSNPTDDDVSSGSSSERSSTSGGYIFYTFSTVHPIPDEDSPWITDSTD | |
| | R | |
| | IPATS | |
| Wild type | atggac aagttttggt ggcacgcagc ctggggactc tgcctcgtgc | 7 |
| CD44 NH₂- | cgctgagcct ggcg cagatc gatttgaata taacctgccg ctttgcaggt gtattccacg | |
| terminal | tggagaaaaa tggtcgctac agcatctctc ggacggaggc cgctgacctc tgcaaggctt | |
| common | tcaatagcac cttgcccaca atggcccaga tggagaaagc tctgagcatc ggatttgaga | |
| extracellular | cctgcaggta tgggttcata gaagggcacg tggtgattcc ccggatccac cccaactcca | |
| domain | tctgtgcagc aaacaacaca ggggtgtaca tcctcacatc caacacctcc cagtatgaca | |
| (exon 1-5) | catattgctt caatgcttca gctccacctg aagaagattg tacatcagtc acagacctgc | |
| | ccaatgcctt tgatggacca attaccataa ctattgttaa ccgtgatggc acccgctatg | |
| | tccagaaagg agaatacaga acgaatcctg aagacatcta ccccagcaac cctactgatg | |
| | atgacgtgag cagcggctcc tccagtgaaa ggagcagcac ttcaggaggt tacatctat | |
| | acaccttttc tactgtacac cccatcccag acgaagacag tccctggatc accgacagca | |
| | cagacagaat ccctgctacc agt | |
| | MDKFWWHAAWGLCLVPLSLAQIDLNITCRFAGVFHVEKNGRYSI | 47 |
| | SRTEAADLCKAFNSTLPTMAQMEKALSIGFETCRYGFIEGHVVIPRIHPNSICAAN | |
| | NT | |
| | GVYILTSNTSQYDTYCFNASAPPEEDCTSVTDLPNAFDGPITITIVNRDGTRYVQK | |
| | GE | |
| | YRTNPEDIYPSNPTDDDVSSGSSSERSSTSGGYIFYTFSTVHPIPDEDSPWITDSTD | |
| | R | |
| | IPATS | |
| Partial | acgtctt caaataccat ctcagcaggc tgggagccaa | |
| CD44v3-v10 | atgaagaaaa tgaagatgaa agagacagac acctcagttt ttctggatca ggcattgatg | 8 |
| extracellular | atgatgaaga ttttatctcc agcacc attt caaccacacc acgggctttt gaccacacaa | |
| domain | aacagaacca ggactggacc cagtggaacc caagccattc aaatccggaa gtgctacttc | |
| (a part of the | agacaaccac aaggatgact gatgtagaca gaaatggcac cactgcttat gaaggaaact | |
| extracellular | ggaacccaga agcacaccct cccctcattc accatgagca tcatgaggaa gaagagaccc | |
| domain | cacattctac aagcacaatc caggcaactc ctagtagtac aacggaagaa acagctaccc | |
| containing | agaaggaaca gtggtttggc aacagatggc atgagggata tcgccaaaca cccaaagaag | |
| exons v3-v10, | actcccattc gacaacaggg acagctgcag cctcagctca taccagccat ccaatgcaag | |
| 16,and 17) | gaaggacaac accaagccca gaggacagtt cctggactga tttcttcaac ccaatctcac | |
| | accccatggg acgaggtcat caagcaggaa gaaggatgga tatggactcc agtcatagta | |
| | taacgcttca gcctactgca aatccaaaca caggtttggt ggaagatttg gacaggacag | |
| | gacctctttc aatgacaacg cagcagagta attctcagag cttctctaca tcacatgaag | |
| | gcttggaaga agataaagac catccaacaa cttctactct gacatcaagc aataggaatg | |
| | atgtcacagg tggaagaaga gacccaaatc attctgaagg ctcaactact ttactggaag | |
| | gttatacctc tcattaccca cacacgaagg aaagcaggac cttcatccca gtgacctcag | |
| | ctaagactgg gtcctttgga gttactgcag ttactgttgg agattccaac tctaatgtca | |
| | atcgttcctt atca ggagac caagacacat tccaccccag tggggggtcc cataccactc | |
| | atggatctga atcagatgga cactcacatg ggagtcaaga aggtggagca aacacaacct | |
| | ctggtcctat aaggacaccc caaattccag aa | |
| | TSSNTISAGWEPNEENEDERDRHLSFSGSGIDDDEDFISSTISTTPRAFDHTK | 48 |
| | QNQDWTQWNPSHSNPEVLLQTTTRMTDVDRNGTTAYEGNWNPEAHPPLIHHE | |
| | HHEEEE | |
| | TPHSTSTIQATPSSTTEETATQICEQWFGNRWHEGYRQTPICEDSHSTTGTAAASA | |
| | HTSH | |
| | PMQGRTTPSPEDSSWTDFFNPISHPMGRGHQAGRRMDMDSSHSITLQPTANPNT | |
| | GLVE | |
| | DLDRTGPLSMTTQQSNSQSFSTSHEGLEEDKDHPTTSTLTSSNRNDVTGGRRDPN | |
| | HSE | |
| | GSTILLEGYTSHYPHTKESRTFIPVTSAKTGSFGVTAVTVGDSNSNVNRSLSGDQ | |
| | DTF | |
| | HPSGGSHTTHGSESDGHSHGSQEGGANTTSGPIRTPQIPE | |
| Partial | ga tatggactcc agtcatagta | 9 |
| CD44v8-v10 | | |
| extracellular | taacgcttca gcctactgca aatccaaaca caggtttggt ggaagatttg gacaggacag | |
| domain | gacctctttc aatgacaacg cagcagagta attctcagag cttctctaca tcacatgaag | |
| (a part of the | gcttggaaga agataaagac catccaacaa cttctactct gacatcaagc aataggaatg | |
| extracellular | atgtcacagg tggaagaaga gacccaaatc attctgaagg ctcaactact ttactggaag | |
| domain | gttatacctc tcattaccca cacacgaagg aaagcaggac cttcatccca gtgacctcag | |
| containing | ctaagactgg gtcctttgga gttactgcag ttactgttgg agattccaac tctaatgtca | |
| exon v8-v10, | atcgttcctt atca ggagac caagacacat tccaccccag tggggggtcc cataccactc | |
| 16 and 17) | atggatctga atcagatgga cactcacatg ggagtcaaga aggtggagca aacacaacct | |
| | ctggtcctat aaggacaccc caaattccag aa | |
| | DMDSSHSITLQPTANPNTGLVE | |
| | DLDRTGPLSMTTQQSNSQSFSTSHEGLEEDKDHPITSTLTSSNRNDVTGGRRDPN | 49 |
| | HSE | |
| | GSTTLLEGYTSHYPHTKESRTFIPVTSAKTGSFGVTAVTVGDSNSNVNRSLSGDQ | |
| | DTF | |
| | HPSGGSHTTHGSESDGHSHGSQEGGANTTSGPIRTPQIPE | |
| CD44s-Fc | atggac aagttttggt ggcacgcagc ctggggactc tgcctcgtgc | 10 |
| (exon 1-5, 16, | cgctgagcct ggcg cagatc gatttgaata taacctgccg ctttgcaggt gtattccacg | |
| and 17 of | tggagaaaaa tggtcgctac agcatctctc ggacggaggc cgctgacctc tgcaaggctt | |
| CD44- | tcaatagcac cttgcccaca atggcccaga tggagaaagc tctgagcatc ggatttgaga | |
| CAATTG | cctgcaggta tgggttcata gaagggcacg tggtgattcc ccggatccac cccaactcca | |
| -Fc) | tctgtgcagc aaacaacaca ggggtgtaca tcctcacatc caacacctcc cagtatgaca | |
| | catattgctt caatgcttca gctccacctg aagaagattg tacatcagtc acagacctgc | |
| | ccaatgcctt tgatggacca attaccataa ctattgttaa ccgtgatggc acccgctatg | |
| | tccagaaagg agaatacaga acgaatcctg aagacatcta ccccagcaac cctactgatg | |
| | atgacgtgag cagcggctcc tccagtgaaa ggagcagcac ttcaggaggt tacatctttt | |
| | acaccttttc tactgtacac cccatcccag acgaagacag tccctggatc accgacagca | |
| | cagacagaat ccctgctacc aga gac caagacacat tccaccccag tggggggtcc cataccactc | |
| | atggatctga atcagatgga cactcacatg ggagtcaaga aggtggagca aacacaacct | |
| | ctggtcctat aaggacaccc caaattccag aa CAATTG | |
| | gacaaaactc acacatgccc accgtgccca gcacctgaac tcctgggggg accgtcagtc ttcctcttcc | |
| | ccccaaaacc caaggacacc ctcatgatct cccggacccc | |
| | tgaggtcaca tgcgtggtgg | |
| | tggacgtgag ccacgaagac cctgaggtca agttcaactg | |
| | gtacgtggac ggcgtggagg | |
| | tgcataatgc caagacaaag ccgcgggagg agcagtacaa cagcacgtac cgtgtggtca | |
| | gcgtcctcac cgtcctgcac caggactggc tgaatggcaa ggagtacaag tgcaaggtct | |
| | ccaacaaagc cctcccagcc cccatcgaga aaaccatctc caaagccaaa gggcagcccc | |
| | gagaaccaca ggtgtacacc ctgcccccat cccgggatga gctgaccaag aaccaggtca | |
| | gcctgacctg cctggtcaaa ggcttctatc ccagcgacat cgccgtggag tgggagagca | |
| | atgggcagcc ggagaacaac tacaagacca cgcctcccgt gctggactcc gacggctcct | |
| | tcttcctcta cagcaagctc accgtggaca agagcaggtg gcagcagggg aacgtcttct | |
| | catgctccgt gatgcatgag gctctgcaca accactacac gcagaagagc ctctccctgt | |
| | ctccgggtaa atga | |
| | MDKFWWHAAWGLCLVPLSLAQIDLNITCRFAGVFHVEKNGRYSI | 50 |
| | SRTEAADLCKAFNSTLPTMAQMEKALSIGFETCRYGFIEGHVVIPRIHPNSICAAN | |
| | NT | |
| | GVYILTSNTSQYDTYCFNASAPPEEDCTSVTDLPNAFDGPITITIVNRDGTRYVQK | |
| | GE | |
| | YRTNPEDIYPSNPTDDDVSSGSSSERSSTSGGYIFYTFSTVHPIPDEDSPWITDSTD | |
| | RIPAT R DQDTF HPSGGSHTTHGSESDGHSHGSQEGGANTTSGPIRTPQIPE | |
| | QL DKTHTCPP CPAPELLGGP SVFLFPPKPK DTLMISRTPE VTCVVVDVSH | |
| | EDPEVKFNWY VDGVEVHNAK TKPREEQYNS TYRVVSVLTV LHQDWLNGKE | |
| | YKCKVSNKAL PAPIEKTISK AKGQPREPQV YTLPPSRDEL TKNQVSLTCL | |
| | VKGFYPSDIA VEWESNGQPE NNYKTTPPVL DSDGSFFLYS KLTVDKSRWQ | |
| | QGNVFSCSVM HEALHNHYTQ KSLSLSPGK | |
| CD44v3-v10-Fc | atggac aagttttggt ggcacgcagc ctggggactc tgcctcgtgc | |
| (exon 1-5, | cgctgagcct ggcgcagatc gatttgaata taacctgccg ctttgcaggt gtattccacg tggagaaaaa tggtcgctac | 11 |
| v3-v10, 16, | agcatctctc ggacggaggc cgctgacctc tgcaaggctt tcaatagcac cttgcccaca atggcccaga | |
| and 17 of | tggagaaagc tctgagcatc ggatttgagacctgcaggta tgggttcata gaagggcacg tggtgattcc ccggatccac | |
| CD44-CAATTG- | cccaactcca tctgtgcagc aaacaacaca ggggtgtaca tcctcacatc caacacctcc cagtatgaca | |
| Fc) | catattgctt caatgatca gctccacctg aagaagattg tacatcagtc acagacctgc ccaatgcctt tgatggacca | |
| CD44v3-v10-Fc | attaccataa ctattgttaa ccgtgatggc acccgctatg tccagaaagg agaatacaga acgaatcctg aagacatcta | |
| | ccccagcaac cctactgatg atgacgtgag cagcggctcc tccagtgaaa ggagcagcac ttcaggaggt tacatctttt | |
| | acaccttttc tactgtacac cccatcccag acgaagacag tccctggatc accgacagca cagacagaat ccctgctacc | |
| | agtacgtctt caaataccat ctcagcaggc tgggagccaa atgaagaaaa tgaagatgaa agagacagac acctcagttt | |
| | ttctggatca ggcattgatg atgatgaaga ttttatctcc agcaccattt caaccacacc acgggctttt gaccacacaa | |
| | aacagaacca ggactggacc cagtggaacc caagccattc aaatccggaa gtgctacttc agacaaccac | |
| | aaggatgact gatgtagaca gaaatggcac cactgcttat gaaggaaact | |
| | ggaacccaga agcacaccct cccctcattc accatgagca tcatgaggaa gaagagaccc cacattctac | |
| | aagcacaatc caggcaactc ctagtagtac aacggaagaa acagctaccc agaaggaaca gtggtttggc | |
| | aacagatggc atgagggata tcgccaaaca cccaaagaag actcccattc gacaacaggg acagctgcag | |
| | cctcagctca taccagccat ccaatgcaag gaaggacaac accaagccca gaggacagtt cctggactga tttcttcaac | |
| | ccaatctcac | |
| | accccatggg acgaggtcat caagcaggaa gaaggatgga tatggactcc agtcatagta taacgcttca | |
| | gcctactgca aatccaaaca caggtttggt ggaagatttg gacaggacag gacctctttc aatgacaacg cagcagagta | |
| | attctcagag cttctctaca tcacatgaag gcttggaaga agataaagac catccaacaa cttctactct gacatcaagc | |
| | aataggaatg atgtcacagg tggaagaaga gacccaaatc attctgaagg ctcaactact ttactggaag | |
| | gttatacctc tcattaccca cacacgaagg aaagcaggac cttcatccca gtgacctcag ctaagactgg gtcctttgga | |
| | gttactgcag ttactgttgg agattccaac tctaatgtca atcgttcctt atca ggagac caagacacat tccaccccag | |
| | tggggggtcc cataccactc atggatctga atcagatgga cactcacatg ggagtcaaga aggtggagca | |
| | aacacaacct ctggtcctat aaggacaccc caaattccag aa CAATTG gacaaaactc | |
| | acacatgccc accgtgccca gcacctgaac tcctgggggg accgtcagtc | |
| | ttcctcttcc ccccaaaacc caaggacacc ctcatgatct cccggacccc | |
| | tgaggtcaca tgcgtggtgg tggacgtgag ccacgaagac cctgaggtca | |
| | agttcaactg gtacgtggac ggcgtggagg tgcataatgc caagacaaag | |
| | ccgcgggagg agcagtacaa cagcacgtac cgtgtggtca gcgtcctcac | |
| | cgtcctgcac caggactggc tgaatggcaa ggagtacaag tgcaaggtct | |
| | ccaacaaagc cctcccagcc cccatcgaga aaaccatctc caaagccaaa | |
| | gggcagcccc gagaaccaca ggtgtacacc ctgcccccat cccgggatga | |
| | gctgaccaag aaccaggtca gcctgacctg cctggtcaaa ggcttctatc | |
| | ccagcgacat cgccgtggag tgggagagca atgggcagcc ggagaacaac | |
| | tacaagacca cgcctcccgt gctggactcc gacggctcct tcttcctcta | |
| | cagcaagctc accgtggaca agagcaggtg gcagcagggg aacgtcttct | |
| | catgctccgt gatgcatgag gctctgcaca accactacac gcagaagagc | |
| | ctctccctgt ctccgggtaa atga | |
| | MDKFWWHAAWGLCLVPLSLAQIDLNITCRFAGVFHVEKNGRYSI | 51 |
| | SRTEAADLCKAFNSTLPTMAQMEICALSIGFETCRYGFIEGHVVIPRIHPNSICAAN | |
| | NT | |
| | GVYILTSNTSQVDTYCFNASAPPEEDCTSVTDLPNAFDGPITITIVNRDGTRYVQK | |
| | GE | |
| | YRTNPEDIYPSNPTDDDVSSGSSSERSSTSGGYIFYTFSTVHPIPDEDSPWITDSTD | |
| | R | |
| | IPATS TSSNTISAGWEPNEENEDERDRHLSFSGSGIDDDEDFISST | |
| | ISTTPRAFDHTK QNQDWTQWNPSHSNPEVLLQTTTRMT | |
| | DVDRNGTTAYEGNWNPEAHPPLIHHEHHEEEE TPHSTSTI | |
| | QATPSSTTEETATQKEQWFGNRWHEGYRQTPICEDSHSTTGTAA | |
| | ASAHTSH PMQGRTTPSPEDSSWTDFFNPISHPMGRGHQAGRRM | |
| | DMDSSHSITLQPTANPNTGLVE DLDRTGPLSMTT | |
| | QQSNSQSFSTSHEGLEEDKDHPTTSTLTSS | |
| | NRNDVTGGRRDPNHSE | |
| | GSTTLLEGYTSHYPHTKESRTFIPVTSAKTGSFGVTAVTVGDSNSNVNRSLS | |
| | GDQDTF HPSGGSHTTHGSESDGHSHGSQEGGANTTSGPIRTPQIPE | |
| | QL DKTHTCPP CPAPELLGGP SVFLFPPKPK DTLMISRTPE VTCVVVDVSH | |
| | EDPEVKFNWY VDGVEVHNAK TKPREEQYNS TYRVVSVLTV LHQDWLNGKE | |
| | YKCKVSNKAL PAPIEKTISK AKGQPREPQV YTLPPSRDEL TKNQVSLTCL | |
| | VKGFYPSDIA VEWESNGQPE NNYKTTPPVL DSDGSFFLYS KLTVDKSRWQ | |
| | QGNVFSCSVM HEALHNHYTQ KSLSLSPGK | |
| CD44v8-v10-FC | Atg gac aagttttggt ggcacgcagc ctggggactc tgcctcgtgc | 12 |
| (exon 1-5, | cgctgagcct ggcg cagatc gatttgaata taacctgccg ctttgcaggt gtattccacg | |
| v8-v10, 16, | tggagaaaaa tggtcgctac agcatctctc ggacggaggc cgctgacctc tgcaaggctt | |
| and 17 of | tcaatagcac cttgcccaca atggcccaga tggagaaagc tctgagcatc ggatttgaga | |
| CD44-CAATTG-Fc | cctgcaggta tgggttcata gaagggcacg tggtgattcc ccggatccac cccaactcca | |
| CD44v8-v10-FC | tctgtgcagc aaacaacaca ggggtgtaca tcctcacatc caacacctcc cagtatgaca | |
| | catattgctt caatgcttca gciccacctg aagaagattg tacatcagtc acagacctgc | |
| | ccaatgcctt tgatggacca attaccataa ctattgttaa ccgtgatggc acccgctatg | |
| | tccagaaagg agaatacaga acgaaicctg aagacatcta ccccagcaac cctactgatg | |
| | atgacgtgag cageggctec tccagtgaaa ggagcagcac ttcaggaggt tacatctttt | |
| | acaccttttc tactgtacac cccatcccag acgaagacag tccctggatc accgacagca | |
| | cagacagaat ccctgctacc agt ga tatggactcc agtcatagta | |
| | taaegcttea gcctactgca aatccaaaca caggittggt ggaagatttg gacaggacag gacctcmc aatgacaacg | |
| | cagcagagia aitctcagag cttctciaca icacatgaag gcriggaaga agataaagac catccaacaa cttctactct | |
| | gacatcaagc aafaggaatg atgtcacagg tggaagaaga gacccaaatc attctgaagg ctcaactact ttactggaag | |
| | gttatacctc tcattaccca cacacgaagg aaagcaggac cttcatccca gtgacctcag | |
| | ctaagactgg gtcctttgga gttactgcag ttactgttgg agattccaac tctaatgtca ategttcett atcaggagac | |
| | caagacacat tccaccccag tggggggtcc cataccactc atggatctga atcagatgga cactcacatg | |
| | ggagtcaaga aggtggagca aacacaacct ctggtcctat aaggacaccc caaattccag aa CAATTG | |
| | gacaaaactc | |
| | acacatgccc accgtgccca gcacctgaac tcctgggggg accgtcagtc | |
| | ttcctcttcc ccccaaaacc caaggacacc ctcatgatct cccggacccc | |
| | tgaggtcaca tgcgtggtgg tggacgtgag ccacgaagac cctgaggtca | |
| | agttcaactg gtacgtggac ggcgtggagg tgcataatgc caagacaaag | |
| | ccgegggagg agcagtacaa cagcacgtac cgtgtggtca gcgtcctcac | |
| | cgtcctgcac caggactggc tgaatggcaa ggagtacaag tgcaaggtct | |
| | ccaacaaagc cctcccagcc cccatcgaga aaaccatctc caaagecaaa | |
| | gggcagcccc gagaaccaca ggtgtacacc ctgcccccat cccgggatga | |
| | gctgaccaag aaccaggtca gcctgacctg cctggtcaaa ggcttcratc | |
| | ccagcgacat cgccgtggag tgggagagca atgggcagcc ggagaacaac | |
| | tacaagacca cgcctcccgt gctggactcc gacggctcct tcttcctcia | |
| | cagcaagctc accgtggaca agagcaggtg gcagcagggg aacgtcttct | |
| | catgctccgt gatgeatgag gctcigcaca accaciacae gcagaagagc | |
| | ctctccctgt ctccgggtaa atga | |
| | MDKFWWHAAWGLCLVPLSLAQIDLNITCRFAGVFHVEKNGRYSI | 52 |
| | SRTEAADLCKAFNSILR1 MAQMEKAli5IGFETCRYGFIEGHVVIPRIHPNSICAAN | |
| | NT | |
| | GVYILTSNTSQYDTYCFNASAPPEEDCTSVTDLPNAFDGPITITIVNRDGTRYVQK | |
| | GE | |
| | YRTNPEDIYPSNPTDDDVSSGSSSERSSTSGGYIFYTFSTVHPIPDEDSPWITDSTD | |
| | R | |
| | IPATS DMDSSHSITLQPTANPNTGLVE DLDRTGPLSMTT | |
| | QQSNSQSFSTSHEGLEEDKDHPTTSTLTSSNRNDVTGGRRDPNMSE | |
| | GSTTLLEGYTSHYPIITKESRTFIPVTSAKTGSFGVTAVTVGDSNSNVNRSLS | |
| | GDQDTF HPSGGSHTTHGSESDGHSHGSQEGGANTTSGPIRTPQIPE | |
| | QL DKTHTCPP CPAPELLGGP SVFLFPPKPK DTLMISRTPE VTCVVVDVSH | |
| | EDPEVKFNWY VDGVEVHNAK IXPREEQYNS TYRVVSVLTV LHQDWLNGKE | |
| | YKCKVSNKAL PAPIFKTISK AKGQPREPQV YTLPPSRDEL TKNQVSLTCL | |
| | VKGFYPSDIA VEWESNGQPE NNYKTTPPVL DSDGSFFLYS KLTVDKSRWQ | |
| | QGNVFSCSVM HEALHNHYTQ KSLSLSPGK | |
| CD44sR41A-Fc | atggac aagttttggt ggcacgcagc ctggggactc tgcctcgtgc cgctgagcct ggcg cagatc gatttgaata | 13 |
| | taacctgccg ctttgcaggt gtattccacg tggagaaaaa t ggt gcc tac agcatctctc ggacggaggc | |
| | cgctgacctc tgcaaggctt tcaatagcac cttgcccaca atggcccaga tggagaaagc tctgagcatc ggatttgaga | |
| | cctgcaggta tgggttcata gaagggcacg tggtgattcc ccggatccac cccaactcca tctgtgcagc | |
| | aaacaacaca ggggtgtaca tcctcacatc caacacctcc cagtatgaca | |
| | catattgctt caatgcttca gctccacctg aagaagattg tacatcagtc acagacctgc ccaatgcctt tgatggacca | |
| | attaccataa ctattgttaa ccgtgatggc acccgctatg tccagaaagg agaatacaga acgaatcctg aagacatcta | |
| | ccccagcaac cctactgatg | |
| | atgacgtgag cagcggctcc tccagtgaaa ggagcagcac ttcaggaggt tacatctttt | |
| | acaccttttc tactgtacac cccatcccag acgaagacag tccctggatc accgacagca | |
| | cagacagaat ccctgctacc aga gac caagacacat tccaccccag tggggggtcc cataccactc | |
| | atggatctga atcagatgga cactcacatg ggagtcaaga aggtggagca aacacaacct | |
| | ctggtcctat aaggacaccc caaattccag aa CAATTG gacaaaactc | |
| | acacatgccc accgtgccca gcacctgaac tcctgggggg | |
| | accgtcagtc ttcctcttcc | |
| | ccccaaaacc caaggacacc ctcatgatct cccggacccc | |
| | tgaggtcaca tgcgtggtgg | |
| | tggacgtgag ccacgaagac cctgaggtca agttcaactg | |
| | gtacgtggac ggcgtggagg | |
| | tgcataatgc caagacaaag ccgcgggagg agcagtacaa cagcacgtac cgtgtggtca | |
| | gcgtcctcac cgtcctgcac caggactggc tgaatggcaa ggagtacaag tgcaaggtct | |
| | ccaacaaagc cctcccagcc cccatcgaga aaaccatctc caaagccaaa gggcagcccc | |
| | gagaaccaca ggtgtacacc ctgcccccat cccgggatga gctgaccaag aaccaggtca | |
| | gcctgacctg cctggtcaaa ggcttctatc ccagcgacat cgccgtggag tgggagagca | |
| | atgggcagcc ggagaacaac tacaagacca cgcctcccgt gctggactcc gacggctcct | |
| | tcttcctcta cagcaagctc accgtggaca agagcaggtg gcagcagggg aacgtcttct | |
| | catgctccgt gatgcatgag gctctgcaca accactacac gcagaagagc ctctccctgt | |
| | ctccgggtaa atga | |
| | MDKFWWHAAWGLCLVPLSLAQIDLNITCR_FAGVFHVEKNGAYSI | 53 |
| | SRTEAADLCKAFNSTLPTMAQMEKALSIGFETCRYGFIEGHVVIPPdHPNSICAAN | |
| | NT | |
| | GVYILTSNTSQYDTYCFNASAPPEEDCTSVTDLPNAFDGPITITIVNRDGTRYVQK | |
| | GE | |
| | YRTNPEDIYPSNPTDDDVSSGSSSERSSTSGGYIFYTFSTVHPIPDEDSPWITDSTD | |
| | RIPAT R DQDTF HPSGGSHTTHGSESDGHSHGSQEGGANTTSGPIRTPQIPE | |
| | QL DKTHTCPP CPAPELLGGP SVFLFPPKPK DTLMISRTPE VTCVVVDVSH | |
| | EDPEVKFNWY VDGVEVHNAK TKPREEQYNS TYRVVSVLTV LHQDWLNGKE | |
| | YKCKVSNKAL PAPIEKTISK AKGQPREPQV YTLPPSRDEL TKNQVSLTCL | |
| | VKGFYPSDIA VEWESNGQPE NNYKTTPPVL DSDGSFFLYS KLTVDKSRWQ | |
| | QGNVFSCSVM HEALHNHYTQ KSLSLSPGK | |
| CD44-Fc with a | Wild type CD44 NH2-terminal common extracellular domain (SEQ ID No. 7, exon 1-5) | 14 |
| MfeI | +/− various CD44 extracellular domains (SEQ ID 8, 9, or 18-30) + CAATTG (GlnLeu) + | |
| restriction | SEQID NO. 1 | |
| enzyme cutting | | |
| site | | |
| CD44-Fc without | Wild type CD44 NH2-terminal common extracellular domain (SEQ ID No. 7) | 15 |
| a MfeI | CD44 extracellular domain (SEQ ID 8, 9, or 18-30) + SEQID NO. 1 | |
| restriction | | |
| enzyme cutting | | |
| site | | |
| CD44R41A-Fc | CD44 NH2-terminal common extracellular domain with R41A mutation (SEQ ID +1906) +/− | 16 |
| with a MfeI | various CD44 extracellular domain (SEQ ID 8, 9, or 18- 30) + CAATTG (GlnLeu) + | |
| restriction | SEQID No. 1 | |
| enxyme cutting | | |
| site | | |
| CD44R41A-Fc | CD44 NH2-terminal common extracellular domain with R41A mutation (SEQ ID NO | 17 |
| without | 6). | |
| restriction | | |
| enzyme cutting | | |
| site | | |
| Partial | attt caaccacacc acgggctttt gaccacacaa | 18 |
| CD44v4-v10 | aacagaacca ggactggacc cagtggaacc caagccattc aaatccggaa gtgctacttc | |
| extrcellular | agacaaccac aaggatgact gatgtagaca gaaatggcac cactgcttat gaaggaaact | |
| domain | ggaacccaga agcacaccct cccctcattc accatgagca tcatgaggaa gaagagaccc | |
| (a part | cacattctac aagcacaatc caggcaactc ctagtagtac aacggaagaa acagctaccc | |
| of the | agaaggaaca gtggtUggc aacagatggc atgagggata tcgccaaaca cccaaagaag | |
| extracellular | actcccattc gacaacaggg acagctgcag cctcagctca taccagccat ccaatgcaag | |
| domain | gaaggacaac accaagccca gaggacagtt cctggactga tttcttcaac ccaatctcac | |
| containing | accccatggg acgaggtcat caagcaggaa gaaggatgga tatggactcc agtcatagta | |
| exon v4-v10,16, | taacgcttca gcctactgca aatccaaaca caggtttggt ggaagatttg gacaggacag | |
| and 17) | gacctctttc aatgacaacg cagcagagta attctcagag cttctctaca tcacatgaag | |
| | gcttggaaga agataaagac catccaacaa cttctactct gacatcaagc aataggaatg | |
| | atgtcacagg tggaagaaga gacccaaatc attctgaagg ctcaactact ttactggaag | |
| | gttatacctc tcattaccca cacacgaagg aaagcaggac cttcatccca gtgacctcag | |
| | ctaagactgg gtcctttgga gttactgcag ttactgttgg agattccaac tctaatgtca | |
| | atcgttcctt atca ggagac caagacacat tccaccccag tggggggtcc cataccactc | |
| | atggatctga atcagatgga cactcacatg ggagtcaaga aggtggagca aacacaacct | |
| | ctggtcctat aaggacaccc caaattccag aa | |
| | ISTTPRAFDHTK | 58 |
| | QNQDWTQWNPSHSNPEVLLQTTTRMTDVDRNGTTAYEGNWNPEAHPPLIHHE | |
| | HHEEEE | |
| | TPHSTSTIQATPSSTTEETATQICEQWEGNRWHEGYRQTPKEDSHSTTGTAAASA | |
| | HTSH | |
| | PMQGRTTPSPEDSSWTDFFNPISHPMGRGHQAGRRMDMDSSHSITLQPTANPNT | |
| | GLVE | |
| | DLDRTGPLSMTTQQSNSQSFSTSHEGLEEDKDHPTTSTLTSSNRNDVIGGRRDPN | |
| | HSE | |
| | GSTILLEGYTSHYPHTKESRTFIPVTSAKTGSFGVTAVTVGDSNSNVNRSLSGDQ | |
| | DTF | |
| | HPSGGSHTTHGSESDGHSHGSQEGGANTTSGPIRTPQIPE | |
| Partial CD44v5- | gatgtagaca gaaatggcac cactgcttat gaaggaaact | 19 |
| v10 | ggaacccaga agcacaccct cccctcattc accatgagca tcatgaggaa gaagagaccc | |
| extracellular | cacattctac aagcacaatc caggcaactc ctagtagtac aacggaagaa acagctaccc | |
| domain | agaaggaaca gtggtttggc aacagatggc atgagggata tcgccaaaca cccaaagaag | |
| (a part of the | actcccattc gacaacaggg acagctgcag cctcagctca taccagccat ccaatgcaag | |
| extracellular | gaaggacaac accaagccca gaggacagtt cctggactga tttcttcaac ccaatctcac | |
| domain | accccatggg acgaggtcat caagcaggaa gaaggatgga tatggactcc agtcatagta | |
| containing | taacgcttca gcctactgca aatccaaaca caggtttggt ggaagatttg gacaggacag | |
| exon v5-v10, | gacctctttc aatgacaacg cagcagagta attctcagag cttctctaca tcacatgaag | |
| 16, and 17) | gcttggaaga agataaagac catccaacaa cttctactct gacatcaagc aataggaatg | |
| | atgtcacagg tggaagaaga gacccaaatc attctgaagg ctcaactact ttactggaag | |
| | gttatacctc tcattaccca cacacgaagg aaagcaggac cttcatccca gtgacctcag | |
| | ctaagactgg gtcctttgga gttactgcag ttactgttgg agattccaac tctaatgtca | |
| | atcgttcctt atca ggagac caagacacat tccaccccag tggggggtcc cataccactc | |
| | atggatctga atcagatgga cactcacatg ggagtcaaga aggtggagca aacacaacct | |
| | ctggtcctat aaggacaccc caaattccag aa | |
| | DVDRNGTTAYEGNWNPEAHPPLIHHEHHEEEE | 59 |
| | TPHSTSTIQATPSSTTEETATQ10EQWFGNRWHEGYRQTPKEDSHSTTGTAAASA | |
| | HTSH | |
| | PMQGRTTPSPEDSSWTDFFNPISHPMGRGHQAGRAMDMDSSHSITLQPTANPNT | |
| | GLVE | |
| | DLDRTGPLSMTTQQSNSQSFSTSHEGLEEDICDHPTTSTLTSSNRNDVTGGRRDPN | |
| | HSE | |
| | GSTTLLEGYTSHYPHTKESRTFIPVTSAKTGSFGVTAVTVGDSNSNVNRSLSGDQ | |
| | DTF | |
| | HPSGGSHITHGSESDGHSHGSQEGGANTTSGPIRTPQIPE | |
| Partial | caggcaactc ctagtagtac aacggaagaa acagctaccc | 20 |
| CD44v6-v10 | agaaggaaca gtggtttggc aacagatggc atgagggata tcgccaaaca cccaaagaag | |
| extracellular | actcccattc gacaacaggg acagctgcag cctcagctca taccagccat ccaatgcaag | |
| domain (a part | gaaggacaac accaagccca gaggacagtt cctggactga tttcttcaac ccaatctcac | |
| of extracellular | accccatggg acgaggtcat caagcaggaa gaaggatgga tatggactcc agtcatagta | |
| domain | taacgcttca gcctactgca aatccaaaca caggtttggt ggaagatttg gacaggacag | |
| containing | gacctctttc aatgacaacg cagcagagta attctcagag cttctctaca tcacatgaag | |
| exon v6-v10, | gcttggaaga agataaagac catccaacaa cttctactct gacatcaagc aataggaatg | |
| 16, and 17) | atgtcacagg tggaagaaga gacccaaatc attctgaagg ctcaactact ttactggaag | |
| | gttatacctc tcattaccca cacacgaagg aaagcaggac cttcatccca gtgacctcag | |
| | ctaagactgg gtcctttgga gttactgcag ttactgttgg agattccaac tctaatgtca | |
| | atcgttcctt atca ggagac caagacacat tccaccccag tggggggtcc cataccactc | |
| | atggatctga atcagatgga cactcacatg ggagtcaaga aggtggagca aacacaacct | |
| | ctggtcctat aaggacaccc caaattccag aa | |
| | QATPSS ITEETATQKEQWFGNRWHEGYRQTPKEDSHSTTGTAAASAHTSH | |
| | PMQGRTTPSPEDSSWTDFFNPISHPMGRGHQAGRAMDMDSSHSITLQPTANPNT | 60 |
| | GLVE | |
| | DLDRTGPLSMTTQQSNSQSFSTSHEGLEEDKDHPTTSTLTSSNRNDVTGGRRDPN | |
| | HSE | |
| | GSTTLLEGYTSHYPHTICESRTFIPVTSAKTGSFGVTAVTVGDSNSNVNRSLSGDQ | |
| | DTF | |
| | HPSGGSHTTHGSESDGHSHGSQEGGANTTSGPIRTPQIPE | |
| Partial | g cctcagctca taccagccat ccaatgcaag | 21 |
| CD44v7-v10 | gaaggacaac accaagccca gaggacagtt cctggactga tttcttcaac ccaatctcac | |
| extracellular | accccatggg acgaggtcat caagcaggaa gaaggatgga tatggactcc agtcatagta | |
| domain | taacgcttca gcctactgca aatccaaaca caggtttggt ggaagatttg gacaggacag | |
| (a part of the | gacctctttc aatgacaacg cagcagagta attctcagag cttctctaca tcacatgaag | |
| extracellular | gcttggaaga agataaagac catccaacaa cttctactct gacatcaagc aataggaatg | |
| domain | atgtcacagg tggaagaaga gacccaaatc attctgaagg ctcaactact ttactggaag | |
| containing | gttatacctc tcattaccca cacacgaagg aaagcaggac cttcatccca gtgacctcag | |
| exon v7- | ctaagactgg gtcctttgga gttactgcag ttactgttgg agattccaac tctaatgtca | |
| v10, 16, | atcgttcctt atca ggagac caagacacat tccaccccag tggggggtcc cataccactc | |
| and 17) | atggatctga atcagatgga cactcacatg ggagtcaaga aggtggagca aacacaacct | |
| | ctggtcctat aaggacaccc caaattccag aa | |
| | ASAHTSH | |
| | PMQGRTTPSPEDSSWTDFFNPISHPMGRGHQAGRRMDMDSSHSITLQPTANPNT | 61 |
| | GLVE | |
| | DLDRTGPLSMTTQQSNSQSFSTSHEGLEEDKDHPTTSTLTSSNRNDVTGGRRDPN | |
| | HSE | |
| | GSTTLLEGYTSHYPHTICESRTFIPVTSAKTGSFGVTAVTVGDSNSNVNRSLSGDQ | |
| | DTF | |
| | HPSGGSHTTHGSESDGHSHGSQEGGANTTSGPIRTPQIPE | |
| Partial | cagcagagta attctcagag cttctctaca tcacatgaag | 22 |
| CD44v9-v10 | gcttggaaga agataaagac catccaacaa cttctactct gacatcaagc aataggaatg | |
| extracellular | atgtcacagg tggaagaaga gacccaaatc attctgaagg ctcaactact ttactggaag | |
| domain | gttatacctc tcattaccca cacacgaagg aaagcaggac cttcatccca gtgacctcag | |
| (a part of the | ctaagactgg gtcctttgga gttactgcag ttactgttgg agattccaac tctaatgtca | |
| extracellular | atcgttcctt atca ggagac caagacacat tccaccccag tggggggtcc cataccactc | |
| domain | atggatctga atcagatgga cactcacatg ggagtcaaga aggtggagca aacacaacct | |
| containing | ctggtcctat aaggacaccc caaattccag aa | |
| exon v9-v10, | QQSNSQSFSTSHEGLEEDKDHPTTSTLTSSNRNDVTGGRRDPNHSE | 62 |
| 16, and 17) | GSTTLLEGYTSHYPHTKESRTFIPVTSAKTGSFGVTAVTVGDSNSNVNRSLSGDQ | |
| | DTF | |
| | HPSGGSHTTHGSESDGHSHGSQEGGANTTSGPIRTPQIPE | |
| Partial CD44v10 | aataggaatg | 23 |
| extracellular | atgtcacagg tggaagaaga gacccaaatc attctgaagg ctcaactact ttactggaag | |
| domain | gttatacctc tcattaccca cacacgaagg aaagcaggac cttcatccca gtgacctcag | |
| (a part of | ctaagactgg gtcctttgga gttactgcag ttactgttgg agattccaac tctaatgtca | |
| extracellular | atcgttcctt atca ggagac caagacacat tccaccccag tggggggtcc cataccactc | |
| domain | atggatctga atcagatgga cactcacatg ggagtcaaga aggtggagca aacacaacct | |
| containing | ctggtcctat aaggacaccc caaattccag aa | |
| exon v10, | NRNDVTGGRRDPNHSE | 63 |
| 16, and | GSTTLLEGYTSHYPHTKESRTFIPVTSAKTGSFGVTAVTVGDSNSNVNRSLSGDQ | |
| 17) | DTF | |
| | HPSGGSHTTHGSESDGHSHGSQEGGANTTSGPIRTPQIPE | |
| Partial CD44v9 | cagcagagta attctcagag cnctctaca tcacatgaag | 24 |
| extracellular | gcttggaaga agataaagac catccaacaa cttctactct gacatcaagc | |
| domain | ggagac caagacacat tccaccccag tggggggtcc cataccactc | |
| (a part of | atggatctga atcagatgga cactcacatg ggagtcaaga aggtggagca aacacaacct | |
| extracellular | ctggtcctat aaggacaccc caaattccag aa | |
| domain | QQSNSQSFSTSHEGLEEDKDHPTTSTLTS GDQDTF | 64 |
| containing | HPSGGSHTTHGSESDGHSHGSQEGGANTTSGPIRTPQIPE | |
| exon v9, | | |
| 16, and 17) | | |
| Partial CD44v8 | ga tatggactcc agtcatagta | 25 |
| extracellular | taacgcttca gcctactgca aatccaaaca caggtttggt ggaagatttg gacaggacag | |
| domain | gacctctttc aatgacaacg | |
| (a part of | ggagac caagacacat tccaccccag tggggggtcc cataccactc | |
| extracellular | atggatctga atcagatgga cactcacatg ggagtcaaga aggtggagca aacacaacct | |
| domain | ctggtcctat aaggacaccc caaattccag aa | |
| containing | DMDSSHSITLQPTANPNTGLVE DLDRTGPLSMTT GDQDTF | 65 |
| exon v8, | HPSGGSHTTHGSESDGHSHGSQEGGANTTSGPIRTPQIPE | |
| 16, and 17) | | |
| Partial CD44v7 | g cctcagctca taccagccat ccaatgcaag gaaggacaac accaagccca gaggacagtt cctggactga | 26 |
| extracellular | tttcttcaac ccaatctcac accccatggg acgaggtcat caagcaggaa gaaggatg | |
| domain | ggagac caagacacat tccaccccag tggggggtcc cataccactc | |
| (a part of | atggatctga atcagatgga cactcacatg ggagtcaaga aggtggagca aacacaacct | |
| extracellular | ctggtcctat aaggacaccc caaattccag aa | |
| domain | ASAHTSH PMQGRTTPSPEDSSWTDFFNPISHPMGRGHQAGRRM | 66 |
| containing | GDQDTFHPSGGSHTTHGSESDGHSHGSQEGGANTTSGPIRTPQIPE | |
| exon v7, | | |
| 16, and 17) | | |
| Partial CD44v6 | caggcaactc ctagtagtac aacggaagaa acagctaccc agaaggaaca gtggtttggc aacagatggc | 27 |
| extracellular | atgagggata tcgccaaaca cccaaagaag actcccattc gacaacaggg acagctgca | |
| domain | ggagac caagacacat tccaccccag tggggggtcc cataccactc atggatctga atcagatgga cactcacatg | |
| (a part of | ggagtcaaga aggtggagca aacacaacct ctggtcctat aaggacaccc caaattccag aa | |
| extracellular | QATPSSTTEETATQKEQWEGNRWHEGYRQTPKEDSHSTTGTAA | 67 |
| domain | GDQDTF HPSGGSHTTHGSESDGHSHGSQEGGANTTSGPIRTPQIPE | |
| containing | | |
| exon v6, | | |
| 16, and 17) | | |
| Partial CD44v5 | gatgtagaca gaaatggcac cactgcttat gaaggaaact | 28 |
| extracellular | ggaacccaga agcacaccct cccctcattc accatgagca tcatgaggaa gaagagaccc cacattctac | |
| domain | aagcacaatc | |
| (a part of | ggagac caagacacat tccaccccag tggggggtcc cataccactc atggatctga atcagatgga cactcacatg | |
| extracellular | ggagtcaaga aggtggagca aacacaacct | |
| domain | ctggtcctat aaggacaccc caaattccag aa | |
| containing | DVDRNGTTAYEGNWNPEAHPPLIHHEHHEEEE TPHSTSTI | 68 |
| exon v5, | GDQDTF HPSGGSHTTHGSESDGHSHGSQEGGANTTSGPIRTPQIPE | |
| 16, and 17) | | |
| Partial CD44v4 | attt caaccacacc acgggctttt gaccacacaa aacagaacca ggactggacc cagtggaacc caagccattc | 29 |
| extracellular | aaatccggaa gtgctacttc agacaaccac aaggatgact ggagac caagacacat tccaccccag tggggggtcc | |
| domain | cataccactc atggatctga atcagatgga cactcacatg ggagtcaaga aggtggagca aacacaacct ctggtcctat | |
| (a part of | aaggacaccc caaattccag aa | |
| extracellular | ISTTPRAFDHTK QNQDWTQWNPSHSNPEVLLQTTTRMT | 69 |
| domain | GDQDTF HPSGGSHTTHGSESDGHSHGSQEGGANTTSGPIRTPQIPE | |
| containing: | | |
| exon v4, | | |
| 16, and 17) | | |
| Partial CD44v3 | acgtctt caaataccat ctcagcaggc tgggagccaa | 30 |
| extracellular | atgaagaaaa tgaagatgaa agagacagac acctcagttt ttctggatca ggcattgatg | |
| domain | atgatgaaga ttttatctcc agcacc | |
| (a part of | ggagac caagacacat tccaccccag tggggggtcc cataccactc atggatctga atcagatgga cactcacatg | |
| extracellular | ggagtcaaga aggtggagca aacacaacct ctggtcctat aaggacaccc caaattccag aa | |
| domain | TSSNTISAGWEPNEENEDERDRHLSFSGSGIDDDEDFISST | 70 |
| containing | GDQDTF HPSGGSHTTHGSESDGHSHGSQEGGANTTSGPIRTPQIPE | |
| exon v3, | | |
| 16, and 17) | | |

A fusion protein (SEQ ID NO: 3) comprising the constant region (Fc) of immunoglobulin heavy constant gamma 1 (SEQ ID NO: 1) and the CD44 signal peptide (SEQ ID NO: 2) can be used as a negative control for the anti-tumor activity of the CD44-Fc fusion proteins. In some embodiments, the CD44 extracellular domain of the CD44 fusion protein comprises fragments of CD44 extracellular domains, which is encoded by different combinations of the exons 1-17 of the human CD44 gene (FIG. 46). In some embodiments, the extracellular domain of CD44 can be any length, which comprises about 50 to about 100, about 100 to about 150, about 100 to about 200, about 100 to about 300, about 100 to about 400, about 100 to about 500, about 100 to about 600, or about 100 to about 651 amino acid residues of the extracellular domain. In some embodiments, the extracellular domain of CD44 comprises modifications of the polypeptide sequence. The modification can be any modification including, but not limited to, mutations, insertions, substitutions, deletions, and the like. In some embodiments, the fragment comprises a mutation of Arg to Ala. In some embodiments, the mutation of Arg to Ala occurs at a position corresponding to position 41 in the full length CD44 protein (an example of which is set forth in SEQ ID NO: 6). One of ordinary skill in the art can determine which modification(s) increase the anti-cancer efficacy of the CD44 fusion proteins when administered as either a single agent and/or in combination with other anti-cancer therapies. One of ordinary skill in the art can do this by, for example, performing in vivo tumor growth and metastasis experiments to determine the anti-cancer efficacy of the modified CD44 fusion proteins when used as a single agent and/or in combination with other anti-cancer therapies.

In another aspect, CD44 fusion proteins comprise different segments of the extracellular domain of wild type CD44 or the R41A CD44 mutant fused to another non-CD44 molecule. In some embodiments, the non-CD44 molecule is a toxin, peptide, polypeptide, small molecule, drug, and the like. In some embodiments, the non-CD44 molecule is a 6-His-tag, GST polypeptide, HA-tag, the constant region (Fc) of human IgG1, or v5-tag. In some embodiments, the proteinase cleavage sites will be put before the tag sequences, so that after purification these tags can be removed by proteolytic cleavage.

Human soluble CD44-Fc (hsCD44) fusion proteins are generated as described in the Material and Methods section of the Examples by fusing the extracellular domain of human CD44s (SEQ ID No. 4), CD44v3-v10 (SEQ ID No. 7+SEQ ID No. 8), CD44v8-v10 (SEQ ID No. 7+SEQ ID No. 9), CD44v6-v10 (SEQ ID No.7+SEQ ID No.20), CD44v3-v10R41A (SEQ ID No. 6+SEQ ID No. 8), CD44v8-v10R41A (SEQ ID No.6+No.9), CD44sR41A (SEQ ID No. 5) to the constant region (Fc) of human IgG1. For Example, full-length CD44v3-v10 variant contains exons 1-5, v3-v10, 16-18, and 20. CD44-v8-v10 contains exons 1-5, v8-v10, 16-18, and 20. R41A mutation abolishes the binding capacity of CD44 to one of its major ligands, hyaluronan (Peach et al., 1993), and all CD44 isoforms contain this residue.

Fusion proteins between other CD44 variants and the constant region (Fc) of human IgG1 can work effectively as potent anti-cancer agents in similar fashion as the ones used in the Examples. These fusion proteins include but are not limited to CD44v4-v10-, CD44-5-v10-, CD44v7-v10-, CD44v9-v10-, CD44v10-, CD44v3-, CD44v4-, CD44v5-, CD44v6-, CD44v7-, CD44v8-, and CD44v9-Fc or above CD44 isoforms containing R41A mutation (Peach et al., 1993), and different combinations and/or modifications of different extracellular domains/exons of CD44 fused to the constant region (Fc) of human IgG1. The modifications can be any modifications including, but not limited to, mutations, insertions, substitutions, deletions, and the like. The CD44 extracellular domain can also be derived from different combinations of exons 1-17, different deletions, mutations, duplication, or multiplication of the different segments of the extracellular domain of CD44.

In some embodiments, the nucleic acid molecule encoding a CD44 fusion protein is operably linked to a promoter. In some embodiments, the promoter can facilitate the expression in a prokaryotic cell and/or eukaryotic cell, including COS-7, CHO, 293, human glioblastoma cells, and other human cancer cells. The promoter can be any promoter that can drive the expression of the nucleic acid molecule. Examples of promoters include, but are not limited to, CMV, SV40, pEF, actin promoter, and the like. In some embodiments, the nucleic acid molecule is DNA or RNA. In some embodiments, the nucleic acid molecule is a virus, vector, or plasmid. In some embodiments, the expression of the nucleic acid molecule is regulated such that it can be turned on or off based on the presence or absence of a regulatory substance. Examples of such a system include, but are not limited to a tetracycline-ON/OFF system.

Soluble recombinant CD44 HA-binding domain (CD44-HABD) was found to block angiogenesis in vivo in chick and mouse and inhibited growth of melanoma and pancreatic adenocarcinoma (Pall et al., 2004). Soluble CD44 inhibits melanoma tumor growth by blocking cell surface CD44 binding to hyaluronic acid (Ahrens et al., 2001). CD44-receptor globulin inhibits lung metastasis of B16F10 murine melanoma metastasis and CD44-receptor globulin contains the extracellular part of CD44s or CD44v10 linked to the constant region of the immunoglobulin kappa light chain (Zawadzki et al., 1998). Soluble CD44s-immunoglobulin fusion protein inhibits in vivo growth of human lymphoma Namalwa (Sy et al., 1992).

These CD44-Fc fusion proteins can be modified to improve efficacy. These modifications include inserting multiple repeated domains containing the different ligand binding sites and by fusing the CD44 extracellular domain to the parts of the other proteins such as the coil-coil domain of angiopoietins, which are known to oligomerize the molecules.

CD44R41A Mutant vs. Wild Type CD44 Fusion

CD44's major ligand is hyaluronan (HA). CD44 has other ligands such as osteopontin (Verhulst et al., 2003; Zhu et al., 2004), fibronectin, collagen types I and IV (Ponta et al., 1998), serglycin, laminin (Naor et al., 1997), MMP-9 (Yu and Stamenkovic, 1999, 2000), MMP-7 (Yu et al., 2002), and fibrin (Alves et al., 2008). CD44 also cooperates with several receptor tyrosine kinases (Orian-Rousseau and Ponta, 2008; Ponta et al., 2003), P-selectin (Alves et al., 2008), E-selectin (Dimitroff et al., 2001; Hidalgo et al., 2007; Katayama et al., 2005), death receptor (DR) (Hauptschein et al., 2005), and membrane-type 1 matrix metalloproteinase (MT1MMP) (Kajita et al., 2001).

The CD44 HA-binding site is located in the NH₂-terminus (residues 21-178). Modification of CD44 by switching R41 to A abolishes the binding capacity of CD44 to HA (Banerji et al., 2007; Peach et al., 1993). Therefore, modification of CD44-Fc fusion proteins by switching R41 to A can result in CD44-Fc fusion proteins which can effectively trap CD44 ligands other than HA. These mutations are also likely to increase the fusion proteins' bioavailability due to reduced sequestering of these fusion proteins by HA in the extracellular matrix (ECM). These R41A mutations may also result in fusion proteins with a greater capacity for trapping other CD44 ligands and CD44 sheddase(s) due to the increased bioavailability of CD44R41A-Fc fusion proteins, which may be particularly important when the interaction between CD44 and these other ligands or CD44 shedding is driving the progression of particular types of cancer at a particular stage and/or after a particular therapeutic treatment. We have shown that the CD44v3-v10R41A-Fc fusion protein retained a substantial level of anti-GBM activity (FIG. 12C-c), supporting the notion of HA-dependent and HA-independent role of CD44 in cancer.

### CD44 Fusion Protein Expression and Purification

The present invention provides an isolated nucleic acid molecule (polynucleotide) encoding a CD44 fusion protein.

In some embodiments, the nucleic acid molecule is a recombinant viral vector. A “recombinant viral vector” refers to a construct, based upon the genome of a virus that can be used as a vehicle for the delivery of nucleic acids encoding proteins, polypeptides, or peptides of interest. Recombinant viral vectors are well known in the art and are widely reported. Recombinant viral vectors include, but are not limited to, retroviral vectors, adenovirus vectors, adeno-associated virus vectors, and lenti-virus vectors, which are prepared using routine methods and starting materials.

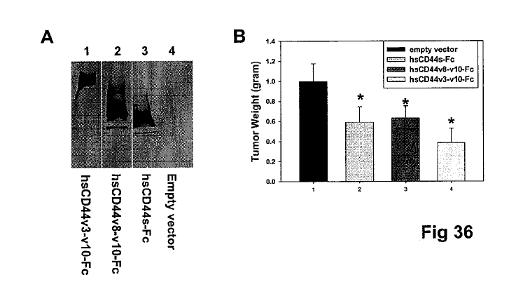

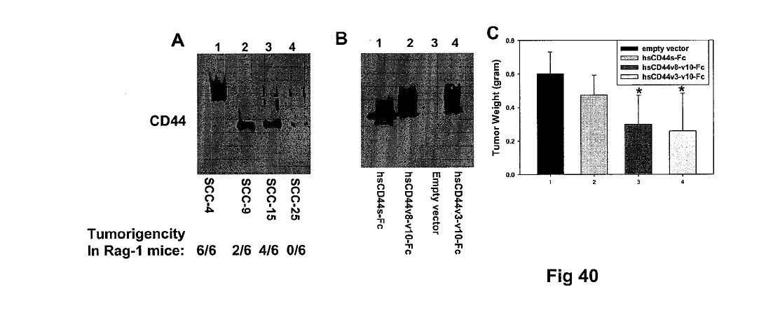

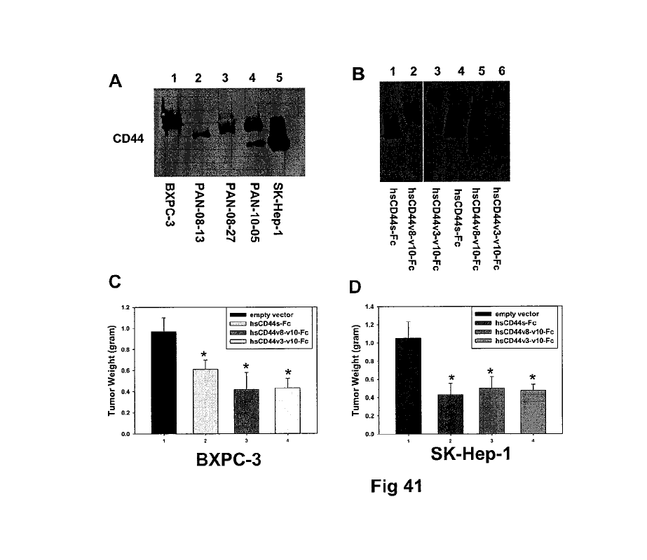

Using standard techniques and readily available starting materials, a nucleic acid molecule may be prepared. The nucleic acid molecule may be incorporated into an expression vector which is then incorporated into a host cell. Host cells for use in well known recombinant expression systems for production of proteins are well known and readily available. Examples of host cells include bacteria cells (e.g. E. coli, yeast cells such as S. cerevisiae), insect cells (e.g., S. frugiperda), non-human mammalian tissue culture cells (e.g., Chinese hamster ovary (CHO) cells and Cos-7 cells), human tissue culture cells (e.g., 293 cells and HeLa cells), glioblastoma cells, and other human cancer cells. All the expression constructs containing nucleic acids encoding CD44 fusion proteins, including CD44-Fc fusion proteins, contain nucleic acids encoding the NH₂-terminal signal peptide of CD44, therefore these CD44 fusion proteins are secreted into cell culture media (FIG. 12A, FIG. 29A, FIG. 36A, FIG. 40B, and FIG. 41B).

Some embodiments involve the insertion of DNA molecules into a commercially available expression vector for use in well-known expression systems. This can be accomplished using techniques known in the art. For example, the commercially available plasmid pSE420 (Invitrogen, San Diego, Calif.) may be used for producing proteins in E. coli. The commercially available plasmid pYES2 (Invitrogen, San Diego, Calif.) may, for example, be used for producing proteins in S. cerevisiae strains of yeast. The commercially available MAXBAC™ complete baculovirus expression system (Invitrogen, San Diego, Calif.) may, for example, be used for producing proteins in insect cells. The commercially available plasmid pcDNAI, pcDNA3, or PEF6/v5-His (Invitrogen, San Diego, Calif.) may, for example, be used for producing proteins in mammalian cells such as Cos-7, CHO, and 293 cells. One having ordinary skill in the art can use these commercial expression vectors and systems or others to produce proteins by routine techniques and readily available starting materials. (See e.g., Sambrook et al., eds., 2001, supra) Thus, the desired proteins or fragments can be prepared in both prokaryotic and eukaryotic systems, resulting in a spectrum of processed forms of the protein or fragments.

One having ordinary skill in the art may use other commercially available expression vectors and systems or produce vectors using well known methods and readily available starting materials. Expression systems containing the requisite control sequences, such as promoters and polyadenylation signals, and preferably enhancers, are readily available and known in the art for a variety of host cells (See e.g., Sambrook et al., eds., 2001).

In some embodiments, the nucleic acid molecules can also be prepared as a genetic construct. “Genetic constructs” include regulatory elements necessary for gene expression of a nucleic acid molecule. The elements include: a promoter, an initiation codon, a stop codon, and a polyadenylation signal. In addition, enhancers can be used for gene expression of the sequence that encodes the protein or fragment. It is necessary that these elements be operably linked to the sequence that encodes the desired polypeptide and that the regulatory elements are operably in the individual or cell to whom they are administered Initiation codons and stop codon are generally considered to be part of a nucleotide sequence that encodes the desired protein. However, it is necessary that these elements are functional in the individual or cell to which the gene construct is administered. The initiation and termination codons must be in frame with the coding sequence. Promoters and polyadenylation signals used must be functional within the cells. Examples of promoters useful to practice the present invention include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metallothionein. Examples of polyadenylation signals useful to practice the present invention include but are not limited to SV40 polyadenylation signals and LTR polyadenylation signals. In some embodiments, the SV40 polyadenylation signal, which is in the pCEP4 plasmid (Invitrogen, San Diego Calif.) referred to as the SV40 polyadenylation signal, is used. In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV. Genetic constructs can be provided with mammalian origin of replication in order to maintain the construct extra chromosomally and produce multiple copies of the construct in the cell. Plasmids pCEP4 and pREP4 from Invitrogen (San Diego, Calif.) contain the Epstein Barr virus origin of replication and nuclear antigen EBNA-1 coding region which produces high copy episomal replication without integration. In some embodiments, the nucleic acid molecule is packaged into infectious viral particles including but not limited to retrovirus, adenovirus, adeno-associated virus, and lenti-virus. In some embodiments, the nucleic acid molecule is free of infectious particles. In some embodiments, the nucleic acid molecule is mixed with and carried by nanoparticles.

CD44 fusion proteins are produced by the cells infected with the expression viral constructs carrying the CD44 fusion cDNA constructs and expressed in the presence of serum free cell culture medium for CD44-Fc fusion proteins or 10% FBS containing medium for all other CD44 fusion proteins. CD44 fusion proteins are purified through affinity columns. For example, CD44-Fc fusion proteins are purified by using protein A column as described (Sy et al., 1992) and soluble CD44 tagged with different epitope tags are purified using affinity column conjugated with the appropriate antibodies.

### Other CD44 Antagonists

In one aspect, the CD44 antagonist is a CD44 fusion protein. In another aspect, the CD44 antagonist is a small molecule. In yet another aspect, the CD44 antagonist is a shRNA or siRNA against human CD44 (SEQ ID No. 31-38). In another aspect, shRNAs and/or siRNAs against human CD44 are administrated in the form of a viral vector with or without being packaged into viral particles. In one aspect, the viral particle is a retrovirus, lentivirus, adenovirus, or adeno-associated virus (AAV). In another aspect, the adenovirus is a replication-impaired, non-integrating, serotype 2, 5, 6, 7, or 8 adenoviral vector. In another aspect, an shRNA against human CD44 is administrated together with other carriers including nanoparticles. In another aspect, an shRNA against human CD44 is administrated alone or in combination with other therapies. In another aspect, a shRNA against human CD44 is administrated prior to or after other anti-cancer therapies, including surgical removal of the tumors.

### DEFINITIONS

The following definitions are provided for clarity and illustrative purposes only, and are not intended to limit the scope of the invention.

### Cancer

“Cancer” refers to abnormal, malignant proliferations of cells originating from epithelial cell tissue (carcinomas), blood cells (leukemias, lymphomas, and myelomas), connective tissue (sarcomas), or glial or supportive cells (gliomas). For example, the present invention described herein may be used for treating or preventing malignancies of the various organ systems, such as those affecting lung, breast, lymphoid, gastrointestinal (e.g., colon), and genitourinary tract, prostate, ovary, pharynx, and nervous system as well as adenocarcinomas which include but are not limited to malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, glioblastoma multiforme (GBM), medulloblastoma, craniopharyngioma, ependymoma, pinealoma, hemangioblastoma, acoustic neuroma, oligodendroglioma, meningioma, melanoma, neuroblastoma, and retinoblastoma, and the like. In one embodiment, the invention relates to the treatment of renal cell carcinoma, mesothelioma, sarcoma, or multiple myeloma. In one embodiment, the invention relates to the treatment of colon cancer. In another embodiment, the invention relates to the treatment of lung cancer. In another embodiment, the invention relates to the treatment of ovarian cancer. In an additional embodiment, the invention relates to the treatment of breast cancer. In another embodiment, the invention relates to the treatment of prostate cancer. In an additional embodiment, the invention relates to the treatment of hepatoma. In an additional embodiment, the invention relates to the treatment of head and neck squamous carcinoma. In an additional embodiment, the invention relates to the treatment of melanoma. In an additional embodiment, the invention relates to the treatment of pancreatic cancer. In yet another embodiment, the invention relates to the treatment of astrocytomas. In a specific embodiment, the invention relates to the treatment of gliomas, including glioblastoma multiforme.

### Cancer Stem Cells

Cancer stem cells (CSCs) or cancer initiating cells (CICs) are a small subset of cancer cells that are capable of self-renewal and have multi-lineage potential. These cells are responsible for the maintenance and repopulation of tumors after therapeutic intervention (Reya et al., 2001). CSCs are also highly resistant to chemo- and radio-therapy, and other forms of cancer therapies. CD44 is a major cell surface marker for many types of CSCs (Stamenkovic and Yu, 2009).

### Anti-Cancer Therapies

The term “anti-cancer therapies” includes, but not limited to, surgery, chemotherapy, radiation therapy, targeted drug therapy, gene therapy, immunotherapy, and combination therapy that combines at least two single therapies to treat cancers and malignancies.

### Radiation Therapy

The term “radiation therapy” or “radiotherapy” refers to use of high-energy radiation to treat cancer. Radiation therapy includes externally administered radiation, e.g., external beam radiation therapy from a linear accelerator, and brachytherapy, in which the source of irradiation is placed close to the surface of the body or within a body cavity. Common radioisotopes used include but are not limited to cesium (¹³⁷Cs), cobalt (⁶⁰Co), iodine (¹³¹I), phosphorus-32 (³²P), gold-198 (¹⁹⁸Au), iridium-192 (¹⁹²Ir), yttrium-90 (⁹⁰Y), and palladium-109 (¹⁰⁹Pd). Radiation is generally measured in Gray units (Gy), where 1 Gy=100 rads.

### Chemotherapy and Targeted Therapy

“Chemotherapy” refers to treatment with anti-cancer drugs. The term encompasses numerous classes of agents including platinum-based drugs, alkylating agents, anti-metabolites, anti-mitotic agents, anti-microtubule agents, plant alkaloids, and anti-tumor antibiotics, kinase inhibitors, proteasome inhibitors, EGFR inhibitors, HER dimerization inhibitors, VEGF inhibitors, antibodies, and antisense nucleotides, siRNA, and shRNAs. Such chemotherapeutic drugs include but are not limited to adriamycin, melphalan, ara-C, carmustine (BCNU), temozolomide, irinotecan, BiCNU, busulfan, CCNU, pentostatin, the platinum-based drugs carboplatin, cisplatin and oxaliplatin, cyclophosphamide, daunorubicin, epirubicin, dacarbazine, 5-fluorouracil (5-FU), leucovorin, fludarabine, hydroxyurea, idarubicin, ifosfamide, methotrexate, altretamine, mithramycin, mitomycin, bleomycin, chlorambucil, mitoxantrone, cytarabine, nitrogen mustard, mercaptopurine, mitozantrone, paclitaxel (Taxol®), docetaxel, topotecan, capecetabine (Xeloda®), raltitrexed, streptozocin, tegafur with uracil, thioguanine, thiotepa, podophyllotoxin, filgristim, profimer sodium, letrozole, amifostine, anastrozole, arsenic trioxide, epithalones A and B tretinioin, leustatin, vinorelbine, vinblastine, vincristine, vindesine, etoposide, gemcitabine, satraplatin, ixabepilone, hexamethylamine, and thalidomide.

Targeted therapeutic agents including but not limited to monoclonal antibodies such as Herceptin® (trastuzumab), Rituxan® (rituximab), Campath® (alemtuzumab), Zevelin® (Ibritumomab, tiuxetan), Alemtuzumab, Gemtuzumab, Bexxar® (Tositumomab), ERBITUX® (Cetuximab), Bevacizumab (Avastin®), Panitumumab (Vectibix®), Gemtuzumab (Mylotarg®). Other targeted therapeutic agents include, but are not limited to, tamoxifen, irinotecan, bortezomib, STI-571 (Gleevac®, Imatinib Mesylate), gefitinib, erlotinib, lapatinib, vandetanib, BIBF1120, pazopanib, neratinib, BIBW2992, CI-1033, PF-2341066, PF-04217903, AMG 208, JNJ-38877605, MGCD-265, SGX-523, GSK1363089, Axitinib, vatalanib, E7080, Sunitinib, Sorafenib, Toceranib, Lestaurtinib, Semaxanib, Cediranib, Nilotinib, Dasatinib, Bosutinib, Lestaurtinib, perifosine, MK-2206, temsirolimus, rapamycin, BEZ235, GDC-0941, PLX-4032, imatinib, AZD0530, bortezomib, XAV-939, advexin (Ad5CMV-p53), Genentech—Compound 8/cIAP-XIAP inhibitor, Abbott Laboratories—Compound 11, interleukins (e.g., 2 and 12) and interferons, e.g., alpha and gamma, huBr-E3, Genasense, Ganite, FIT-3 ligand, MLN491RL, MLN2704, MLN576, and MLN518. Antiangiogenic agents include, but are not limited to, BMS-275291, Dalteparin (Fragmin®) 2-methoxyestradiol (2-ME), thalodmide, CC-5013 (thalidomide analog), maspin, combretastatin A4 phosphate, LY317615, soy isoflavone (genistein; soy protein isolate), AE-941 (Neovastat™; GW786034), anti-VEGF antibody (Bevacizumab; Avastin™), PTK787/ZK 222584, VEGF-trap, ZD6474, EMD 121974, anti-αvβ3 integrin antibody (Medi-522; Vitaxin™), carboxyamidotriazole (CAI), celecoxib (Celebrex®), halofuginone hydrobromide (Tempostatin™), and Rofecoxib (VIOXX®).

The term “gene therapy” includes administration of a vector encoding for a CD44 fusion protein. In some embodiments, the vector carries shRNAs against human CD44 (SEQ ID No.31-38). In some embodiments, the vector is packaged into infectious viral particles including, but not limited to, retrovirus, adenovirus, adeno-associated virus, and lenti-virus. In some embodiments, the vector is free of infectious particles. In some embodiments, the vector is mixed with and carried by nanoparticles. Gene therapy can include the nucleotides encoding CD44-Fc fusion, antisense nucleotides, siRNA, and shRNAs against human CD44.

### Expression Construct

The term “expression construct” means a nucleic acid sequence comprising a target nucleic acid sequence or sequences whose expression is desired, operatively associated with expression control sequence elements which provide for the proper transcription and translation of the target nucleic acid sequence(s) within the chosen host cells. Such sequence elements may include a promoter, an initiation codon, a stop codon, and a polyadenylation signal. In addition, enhancers can be used for gene expression of the sequence that encodes the protein or fragment. The “expression construct” may further comprise “vector sequences.” By “vector sequences” is meant any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes.

Expression constructs of the present invention may comprise vector sequences that facilitate the cloning and propagation of the expression constructs. A large number of vectors, including plasmid, fungal, viral vectors, have been described for replication and/or expression in a variety of eukaryotic and prokaryotic host cells. Standard vectors useful in the current invention are well known in the art and include (but are not limited to) plasmids, cosmids, phage vectors, viral vectors, and yeast artificial chromosomes. The vector sequences may contain a replication origin for propagation in Escherichia coli (E. coli); the SV40 origin of replication; an ampicillin, neomycin, puromycin, hygromycin, and blasticidin resistance gene for selection in host cells; and/or genes (e.g., CD44-Fc fusion gene) that amplify the dominant selectable marker plus the gene of interest. Suitable vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Sambrook et al., eds., 2001, Meth. Enzymol., Vol. 185, Goeddel, ed. (Academic Press, Inc., 1990); Jolly, Canc. Gene Ther. 1:51-64, 1994; Flotte, J. Bioenerg. Biomemb. 25:37-42, 1993; Kirshenbaum et al., J. Clin. Invest. 92:381-387, 1993).

### Express and Expression

The terms “express” and “expression” mean allowing or causing the information in a gene or DNA sequence to become manifest, for example producing a protein by activating the cellular functions involved in transcription, translation, and post-translational modification of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

### Transduction

The term “transduction” means the introduction of a “foreign” nucleic acid (i.e. extrinsic or extracellular gene, DNA or RNA sequence) in a viral expression vector that has been packaged in a retro- or lenti-virus into a cell. Common techniques in molecular biology are use to achieve virus transduction to the appropriate cells. In one aspect, the cells are Cos-7 and 293 cells. In another aspect the cells are human GBM cells, human colon cancer cells, human prostate cancer cells, human breast cancer cells, human melanoma cells, human lung cancer cells, human ovarian cancer cells, human malignant mesothelioma cells, human sarcoma cells, human pancreatic cancer cells, human hepatoma cells, human head and neck squamous carcinoma cells, and human multiple myeloma cells.

### Gene

The term “gene” means a DNA sequence that codes for or corresponds to a particular sequence of amino acids which comprise all or part of one or more proteins or enzymes, and may or may not include regulatory DNA sequences, such as promoter and enhancer sequences, which determine for example the conditions under which the gene is expressed.

A coding sequence is “under the control of” or “operatively associated with” expression control sequences in a cell when RNA polymerase transcribes the coding sequence into RNA, particularly mRNA, which is then trans-RNA spliced (if it contains introns) and translated into the protein encoded by the coding sequence.

The term “expression control sequence” refers to a promoter and any enhancer or suppression elements that combine to regulate the transcription of a coding sequence. In a preferred embodiment, the element is an origin of replication.

Antisense Nucleotides, siRNA, and shRNA

Antisense nucleotides are strings of RNA or DNA that are complementary to “sense” strands of nucleotides. They bind to and inactivate these sense strands. shRNAs are used to silence gene expression. Antisense nucleotides can be used in gene therapy.

Small interfering RNA (siRNA) is a class of double-stranded RNA molecules, 20-25 nucleotides in length with 2-nucleotides 3′ overhangs on either end. siRNA functions in RNA interference (RNAi) pathway, in which it interferes with the expression of a specific gene.

A small or short hairpin RNA (shRNA) is a sequence of RNA that forms a tight hairpin turn that can be used to silence gene expression via RNA interference. A shRNA usually contains two inverted repeat sequences derived from its target gene to form sense and antisense strand in a hairpin, which are separated by a short spacer sequence that form a loop in shRNA and ended with a string of T's that served as a transcription termination site. This design produces an RNA transcript that is predicted to fold into a short hairpin RNA. shRNA is introduced into cells using a vector including viral vectors, which can be package into viral particles. The vector carrying shRNAs drives their transcription by U6, H1, or CMV (pGIPZ for shRNAmir from Open Biosystems) promoter. shRNAmir stands for microRNA-adapted shRNA. This vector is usually passed on to daughter cells, allowing the gene silencing to be inherited. The shRNA hairpin is cleaved by the cellular machinery into siRNA, which is then bound to the RNA-induced silencing complex (RISC). This complex binds to and cleaves target mRNAs to achieve silencing effect. siRNA and shRNA in a vector or packaged in a virus can be used in gene therapy to knock down the expression of a gene including that of CD44.

### About or Approximately

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term ‘about’ means within an acceptable error range for the particular value.

### Include or Comprise

As used herein, the terms “include” and “comprise” are used synonymously. It should be understood that the terms “a” and “an” as used herein refer to “one or more” of the enumerated components. The use of the alternative (e.g., “or”) should be understood to mean either one, both, or any combination thereof of the alternatives.

### Isolated

As used herein, the term “isolated” means that the referenced material is removed from the environment in which it is normally found. Thus, an isolated biological material can be free of cellular components, i.e., components of the cells in which the material is found or produced. Isolated nucleic acid molecules include, for example, a PCR product, an isolated mRNA, a cDNA, or a restriction fragment. Isolated nucleic acid molecules also include, for example, sequences inserted into plasmids, cosmids, artificial chromosomes, and the like. An isolated nucleic acid molecule is preferably excised from the genome in which it may be found, and may or may not be joined to non-regulatory sequences, non-coding sequences, or to other genes located upstream or downstream of the nucleic acid molecule when found within the genome. An isolated protein may or may not be associated with other proteins or nucleic acids, or both, with which it associates in the cell, or with cellular membranes if it is a membrane-associated protein.

### Purified

The term “purified” as used herein refers to material that has been isolated under conditions that reduce or eliminate the presence of unrelated materials, i.e. contaminants, including native materials from which the material is obtained. The isolated material is preferably substantially free of cell or culture components, including tissue culture components, contaminants, and the like. As used herein, the term “substantially free” is used operationally, in the context of analytical testing of the material. Preferably, purified material substantially free of contaminants is at least 50% pure or 60%, 70%, 80% pure, more preferably, 90% pure, and more preferably still at least 99% pure. Purity can be evaluated by chromatography, gel electrophoresis, immunoassay, composition analysis, biological assay, and other methods known in the art.

### Nucleic Acid Molecule

A “nucleic acid molecule” or “oligonucleotide” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3rd ed. Cold Spring Harbor Laboratory Press: Cold Spring Harbor, N.Y.; Sambrook et al., (1989) Molecular Cloning: A Laboratory Manual. 2nd ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4th ed. Wiley-Liss; among others. The Current Protocols listed above are updated several times every year.

### CD44 Fusion Compositions

In certain embodiments, the present invention relates to pharmaceutical compositions for treating or preventing glioma or other cancer types in a mammal. In yet another embodiment, the present invention relates to pharmaceutical compositions for targeting a variety of cancer stem cells in a mammal. In another embodiment, the invention is further directed to pharmaceutical compositions for sensitizing a variety of cancer cells and cancer stem cells including glioma cells to radiation, cytotoxic, and targeted therapeutic stresses for the treatment of gliomas or other cancer types. In another embodiment, the pharmaceutical composition comprises CD44 fusion proteins, acting as CD44 antagonists for the treatment or prevention of a glioma or other cancer types. In another embodiment, the pharmaceutical composition comprises a CD44-Fc fusion protein with a constant region of human IgG1 fused to an extracellular domain of CD44. In another embodiment, the pharmaceutical composition comprises a CD44-Fc fusion protein with a constant region of human IgG1 fused to the CD44 extracellular domain of CD44s, CD44v2-v10, CD44v3-v10, CD44v8-v10, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, CD44v2, CD44sR41A, CD44v2-v10R41A, CD44v3-v10R41A, CD44v8-v10R41A, CD44v4-v10R41A, CD44v5-v10R41A, CD44v6-v10R41A, CD44v7-v10R41A, CD44v9-v10R41A, CD44v10R41A, CD44v9R41A, CD44v8R41A, CD44v7R41A, CD44v6R41A, CD44v5R41A, CD44v4R41A, CD44v3R41A, and CD44v2R41A. In another aspect, CD44 fusion protein comprises different segments of the extracellular domain of wild type CD44 or R41A CD44 mutant as described above fused to another non-CD44 molecule. In some embodiments, the non-CD44 molecule is a toxin, peptide, polypeptide, a small molecule, drug, and the like. In some embodiments, the non-CD44 molecule is a 6-His-tag, GST polypeptide, HA-tag, or v5-tag. In some embodiments, proteinase cleavage sites will be put before the tag sequences, so that after purification these tags can be removed by proteolytic cleavage. For example, the HRV 3C (human rhinovirus type 14 3C) protease cleavage site (LEVLFQ↓GP) can be located before the COOH-terminal v5 and His epitope tags. The HRV 3C protease specifically cleaves the sequence LEVLFQ↓GP at 40° C. and were used to efficiently removal the COOH-terminal tags (Novagen).

A pharmaceutical composition of a CD44 antagonist is in one embodiment a purified CD44 fusion protein, including a purified CD44-Fc fusion protein. In another embodiment the pharmaceutical composition is a virus carrying a CD44 fusion protein, including a CD44-Fc fusion protein. In yet another embodiment the pharmaceutical composition is a siRNA/shRNA against human CD44 (e.g., SEQ ID No. 34, 35, and 36). In an additional embodiment the pharmaceutical composition is a small molecule which antagonizes CD44 function.

In some embodiments the glioma is an astrocytoma. In other embodiments the glioma is a glioblastoma multiforme. In other embodiments, the cancer types are colon cancer, breast cancer, prostate cancer, lung cancer, ovarian cancer, pancreatic cancer, melanoma, malignant mesothelioma, sarcoma, kidney cancer, GI track cancer, pancreatic cancer, hepatoma, head and neck squamous carcinoma, and multiple myeloma.

When formulated in a pharmaceutical composition, a therapeutic compound of the present invention can be admixed with a pharmaceutically acceptable carrier or excipient. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human.

While it is possible to use a composition provided by the present invention for therapy as is, it may be preferable to administer it in a pharmaceutical formulation, e.g., in admixture with a suitable pharmaceutical excipient, diluent, or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent, and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine.

The term “carrier” refers to a diluent, adjuvant, excipient, or vehicle with which the compound is administered. Such pharmaceutical carriers can be sterile liquids, such as saline solution, water, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin (1990, Mack Publishing Co., Easton, Pa. 18042).

Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants, preserving, wetting, emulsifying, and dispersing agents. The pharmaceutical compositions may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

In one embodiment, the pharmaceutical composition is administered as a liquid oral formulation. Other oral dosage forms are well known in the art and include tablets, caplets, gelcaps, capsules, pellets, and medical foods. Tablets, for example, can be made by well-known compression techniques using wet, dry, or fluidized bed granulation methods.

Such oral formulations may be presented for use in a conventional manner with the aid of one or more suitable excipients, diluents, and carriers. Pharmaceutically acceptable excipients assist or make possible the formation of a dosage form for a bioactive material and include diluents, binding agents, lubricants, glidants, disintegrants, coloring agents, and other ingredients. Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used. An excipient is pharmaceutically acceptable if, in addition to performing its desired function, it is non-toxic, well tolerated upon ingestion, and does not interfere with absorption of bioactive materials.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

In one embodiment, the pharmaceutical compositions of CD44 antagonists are CD44 fusion proteins. In another embodiment, the pharmaceutical compositions of CD44 antagonists are viruses carrying an expression vector encoding CD44 fusion proteins. In another embodiment, the pharmaceutical compositions are vectors carrying shRNAs against human CD44 with or without being packaged into viral particles. In one aspect, the viral particle is a retrovirus, lentivirus, adenovirus, or adeno-associated virus (AAV). In another aspect, the adenovirus is a replication-impaired, non-integrating, serotype 2, 5, 6, 7, or 8 adenoviral vector.

In another embodiment, the pharmaceutical composition is administered intravenously or intraperitoneal. In yet another embodiment, the pharmaceutical composition is administered by filling a cavity/space left after removal of a tumor with gel matrix-gallocyanine formulations mixed with the pharmaceutical composition.

In certain embodiments, the present invention relates to methods of detecting CD44 ligands, including HA, by using CD44-Fc fusion proteins. These methods are useful for the diagnosis and prognosis of cancer and for the assessment of therapeutic responses of patients.

### Method of Treatment

The present invention described herein can be used to treat cancer or malignancies. In one embodiment, the invention relates to the treatment of prostate cancer, colon cancer, breast cancer, lung cancer, melanoma, head-neck cancer, liver cancer, pancreatic cancer, and ovarian cancer using CD44 fusion compositions alone or in combinations with radiation, chemotherapy, or targeted therapy as defined herein. In another embodiment, the invention relates to the treatment of astrocytomas using CD44 fusion compositions alone or in combinations with radiation, chemotherapy, or targeted therapy. In yet another embodiment, the invention relates to the treatment of malignant mesothelioma, sarcoma, and multiple myeloma using CD44 fusion compositions alone or in combinations with radiation, chemotherapy, or targeted therapy. In a specific embodiment, the invention relates to the treatment of glioblastoma multiforme using CD44 fusion compositions alone or in combinations with radiation, chemotherapy, or targeted therapy. In another specific embodiment, the invention relates to the treatment of glioblastoma multiforme and other cancer types using CD44 fusion compositions alone or in combinations with carmustine (BCNU), temozolomide, docetaxel, carboplatin, cisplatin, epirubicin, oxaliplatin, cyclophosphamide, methotrexate, fluorouracil, vinblastine, vincristine, leucovorin, mitoxantrone, satraplatin, ixabepilone, pacitaxel, gemcitabine, capecitabine, doxorubicin, etoposide, melphalan, hexamethylamine, irinotecan, topotecan, Herceptin® (trastuzumab), ERBITUX® (Cetuximab), Panitumumab (Vectibix®), Bevacizumab (Avastin®), gefitinib, erlotinib, lapatinib, vandetanib, neratinib, BIBW2992, CI-1033, PF-2341066, PF-04217903, AMG 208, JNJ-38877605, MGCD-265, SGX-523, GSK1363089, Sunitinib, Sorafenib, vandetanib, BIBF1120, pazopanib, vatalanib, axitinib, E7080, perifosine, MK-2206, temsirolimus, rapamycin, BEZ235, GDC-0941, PLX-4032, imatinib, AZD0530, bortezomib, XAV-939, cIAP/XIAP inhibitors such as Compound 8 (Genentech)) (Zobel et al., 2006) and Compound 11 (Abbott Laboratories) (Oost et al., 2004), or advexin (Ad5CMV-p53).

“Treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, and/or (3) relieving the disease, i.e., causing regression of the state, disorder or condition or at least one of its clinical or subclinical symptoms.

An “effective amount” is defined herein in relation to the treatment of cancers is an amount that will decrease, reduce, inhibit, or otherwise abrogate the growth of a cancer cell or tumor by at least 10%, at least 20%, at least 30%, at least 40%, at least 50%, at least 60%, at least 70%, at least 80%, at least 90%, at least 95%, or at least 99%. Thus, an “effective amount” is the quantity of compound in which a beneficial clinical outcome is achieved when the compound is administered to a subject with a cancer. A “beneficial clinical outcome” includes, for example, a reduction in tumor mass, a reduction in metastasis, a reduction in the severity of the symptoms associated with the cancer and/or an increase in the longevity of the mammal compared with the absence of the treatment. It will be appreciated that the amount of CD44 fusion proteins of the invention alone and/or in combinations with chemotherapy or targeted therapy required for use in treatment will vary with the route of administration, the nature of the condition for which treatment is required, and the age, body weight and condition of the patient, and will be ultimately at the discretion of the attendant physician or veterinarian. These compositions will typically contain an effective amount of the compositions of the invention, alone or in combination with an effective amount of any radiation, chemotherapy, or other targeted therapies. Preliminary doses can be determined according to animal tests, and the scaling of dosages for human administration can be performed according to art-accepted practices.

The benefit to an individual to be treated is either statistically significant or at least perceptible to the patient or to the physician.

The present invention provides for the use of the pharmaceutical compositions containing CD44 antagonist, such as CD44 fusion proteins, in combination with other anti-cancer therapies, such as but not limited to, surgery, chemotherapy, radiation therapy, targeted therapy, and immunotherapy to treat cancers and malignancies. In a particular embodiment of the present invention, when combined with other anti-cancer therapies, results in a synergistic treatment of the cancer.

The present invention is further directed to pharmaceutical compositions and methods for sensitizing glioma and other cancer cells to cytotoxic or targeted therapeutic stresses for the treatment of gliomas and other cancer types. In one aspect compositions of the present invention are administered prior to, simultaneously with, or after other anti-cancer therapies. In another aspect compositions of the present invention are administered prior to or simultaneously with, or after a treatment which causes oxidative or cytotoxic stresses. In one particular embodiment of the invention, the stresses are caused by radiation therapy. In another particular embodiment of the invention, the stresses are caused by chemotherapy. In another aspect compositions of the present invention are administered after surgical removal of tumors.

In one aspect, the pharmaceutical compositions of CD44 antagonist, such as CD44 fusion proteins, alone or in combinations with radiation, chemotherapy, or other targeted therapies, are mixed with gel matrix-gallocyanine formulations and administered by filling a cavity/space left after surgical removal of a tumor, including a glioma. In another aspect, viruses carrying a viral expression construct of CD44 fusion proteins or shRNAs against human CD44, are mixed with gel matrix-gallocyanine formulations, alone or in combination with radiation, chemotherapy or other targeted therapies, and administered to a mammal by filling the cavity/space left after surgical removal of a tumor, including a glioma.

In one embodiment, the pharmaceutical composition is administered by percutaneous injection or intralesional injection to tumor lesions, residual tumor lesions, or adjacent normal tissues at the surgical edge following a surgical procedure. In another embodiment, an initial intratumoral stereotactic injection of the pharmaceutical composition is administered 10 minutes on day 1. Patients then undergo tumor resection and receive a series of 1-minute injections of the pharmaceutical composition into the resected tumor cavity wall on day 4. In one embodiment, the pharmaceutical composition is administered by intralesional injection around or near cancer tissues that cannot be surgically removed.

For lung cancer, the pharmaceutical composition is administrated by bronchioalveolar lavage or injected directly into an endobronchial lesion via bronchoscopy or into locoregional tumors via multiple percutaneous punctures under fluoroscopic, ultrasonic, or CT scan guidance. In one embodiment, the pharmaceutical composition is delivered to cancer lesions by CD34+ bone marrow progenitor cells, mesenchyal stem cells, or other adult stem cells or induced pluripotent stem cells transduced to express the pharmaceutical composition. In one embodiment, the pharmaceutical composition is administered prior to, together with, or after chemotherapy, radiation therapy, and other targeted therapy.

### Administration and Dosages

The CD44 fusion proteins and formulations of the present invention can be administered topically, parenterally, orally, by inhalation, as a suppository, or by other methods known in the art. The term “parenteral” includes injection (for example, intravenous, intraperitoneal, epidural, intrathecal, intramuscular, intraluminal, intratracheal, subcutaneous, intralesional, or intratumoral).

Administration of the compositions of the invention may be once a day, twice a day, or more often, but frequency may be decreased during a maintenance phase of the disease or disorder, e.g., once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the present compounds. For example, the present invention can be administered intravenously or intraperitoneally about 1-3 every week at 15 mg/kg.

Keeping the above description in mind, typical dosages of CD44 fusion proteins may range from about 10 mg/kg to about 30 mg/kg. A preferred dose range is on the order of about 10 mg/kg to about 15 mg/kg. In certain embodiments, a patient may receive, for example, once per day intravenously or intraperitoneally for 8 days each month, twice a week, or once a week.

Keeping the above description in mind, typical dosages of viruses carrying expression vectors encoding for CD44 fusion proteins or shRNAs against human CD44 may range from about 5×10e⁹ cfu to about 10×10e¹⁰ cfu. In certain embodiments, a patient may receive a dose of viruses, for example, by intravenous, intratumoral, or peritumoral injection once or twice a week.

Keeping the above description in mind, typical dosages of BCNU may range from about 50 mg/m² to about 200 mg/m² given iv on 3 successive days and this course being repeated at intervals of 6 weeks (Pinkerton and Rana, 1976). A preferred dose range is on the order of about 100 mg/m² to about 150 mg/m² given iv on 3 successive days and this course being repeated at intervals of 6 weeks.

Keeping the above description in mind, typical dosages of TMZ may range from about 50 mg/m² to about 200 mg/m² once daily by intravenous infusion over 90 minutes or the oral capsule formulation. A preferred dose range is on the order of about 75 mg/m² to about 150 mg/m² once daily. In certain embodiments, a patient may receive TMZ, for example, once per day intravenously for 5 days each month (http://www.cancer.gov/cancertopics/druginfo/fda-temozolomide).

Keeping the above description in mind, typical dosages of docetaxel may range from about 50 mg/m² to about 200 mg/m². A preferred dose range is on the order of about 60 mg/m² to about 100 mg/m². In certain embodiments, a patient may receive docetaxel, for example, iv infusion once every three weeks (http://www.drugs.com/ppa/docetaxel.html).

Keeping the above description in mind, typical dosages of carboplatin may range from about 200 mg/m² to about 400 mg/m². A preferred dose range is on the order of about 300 mg/m² to about 400 mg/m². In certain embodiments, a patient may receive carboplatin, for example, once intravenously for every four weeks (http://www.drugs.com/pro/carboplatin.html#DA).

Keeping the above description in mind, typical dosages of cisplatin may range from about 20 mg/m² to about 120 mg/m². A preferred dose range is on the order of about 75 mg to about 100 mg. In certain embodiments, a patient may receive cisplatin, for example, once intravenously per day for 5 days every 3 wk for 3 courses.

Keeping the above description in mind, typical dosages of cyclophosphamide may range from about 1 mg/kg/day to about 5 mg/kg/day. A preferred dose range is on the order of about 2 mg/kg/day to about 5 mg/kg/day. In certain embodiments, a patient may receive cyclophosphamide, for example, once per day intravenously or orally.

Keeping the above description in mind, typical dosages of fluorouracil may range from about 12 mg/kg to about 400 mg. A preferred dose range is on the order of about 15 mg to about 100 mg. In certain embodiments, a patient may receive fluorouracil, for example, once of per day intravenously for 4 successive days

Keeping the above description in mind, typical dosages of mitoxantrone may range from about 10 mg/m² to about 20 mg/m² given as a short iv infusion. A preferred dose range is on the order of about 12 mg/m² to about 14 mg/m². In certain embodiments, a patient may receive mitoxantrone, for example, once intravenously every 21 days.

Keeping the above description in mind, typical dosages of pacitaxel may range from about 3 hours at a dose of 100 mg/m² to about 200 mg/m². A preferred dose range is on the order of about 3 hours at a dose of 175 mg/m². In certain embodiments, a patient may receive pacitaxel, for example, once intravenously every three months.

Keeping the above description in mind, typical dosages of topotecan may range from about 0.5 mg/m² to about 2.5 mg/m² daily. Topotecan can be administered by iv infused over 30 min or taking orally. A preferred dose range is on the order of about 0.75 mg/m²-mg/m²/d.

Keeping the above description in mind, typical dosages of trastuzumab may range from about 2 mg/kg/week to about 8 mg/kg/week. A preferred dose range is on the order of about 2 mg/kg to about 4 mg/kg. In certain embodiments, a patient may receive trastuzumab, for example, one intravenously every week or every three weeks (http://www.drugs.com/ppa/trastuzumab.html).

Keeping the above description in mind, typical dosages of cetuximab may range from about 200 mg/m² to about 400 mg/m². A preferred dose range is on the order of about 250 mg/m² to about 300 mg/m². In certain embodiments, a patient may receive cetuximab, for example, once intravenously every week (http://www.drugs.com/ppa/cetuximab.html).

Keeping the above description in mind, typical dosages of panitumumab may range from about 2 mg/kg to about 10 mg/kg. A preferred dose range is on the order of about 5 mg/kg to about 6 mg/kg. In certain embodiments, a patient may receive panitumumab, for example, once intravenously every 14 days.

Keeping the above description in mind, typical dosages of gefitinib may range from about 100 mg to about 400 mg. A preferred dose range is on the order of about 250 mg. In certain embodiments, a patient may receive gefitinib, for example, one 250 mg tablet daily.

Keeping the above description in mind, typical dosages of erlotinib may range from about 25 mg to about 300 mg. A preferred dose range is on the order of about 100 mg to about 150 mg. In certain embodiments, a patient may receive, for example erlotinib, one tablet per day orally.

Keeping the above description in mind, typical dosages of lapatinib may range from about 1000 mg/day to about 3000 mg/day. A preferred dose range is on the order of about 1250 mg/day to about 1500 mg/day. In certain embodiments, a patient may receive lapatinib, for example, one tablet per day orally.

Keeping the above description in mind, typical dosages of BIBW2992 may range from about 20 mg/day to about 100 mg/day. A preferred dose range is on the order of about 50 mg/day to about 70 mg/day. In certain embodiments, a patient may receive BIBW2992, for example, once a day orally for 14 days and 14 days off for 4 weeks (Eskens et al., 2008).

Keeping the above description in mind, typical dosages of CI-1033 may range from about 50 mg/day to about 200 mg/day. A preferred dose range is on the order of about 100 mg/day to about 150 mg/day. In certain embodiments, a patient may receive CI-1033, for example, once orally over 21 consecutive days of a 28-day cycle (Campos et al., 2005; Nemunaitis et al., 2005).

Keeping the above description in mind, typical dosages of PF-2341066 may range from about 5 mg/kg/day to about 50 mg/kg/day. A preferred dose range is on the order of about 20 mg/kg/day to about 30 mg/kg/day. In certain embodiments, a patient may receive PF-2341066, for example, once per day orally (Zou et al., 2007).

Keeping the above description in mind, typical dosages of sunitinib may range from about 12 mg to about 80 mg. A preferred dose range is on the order of about 40 mg to about 50 mg. In certain embodiments, a patient may receive sunitinib, for example, once per day on a schedule of 4 wk on treatment followed by 2 wk off treatment.

Keeping the above description in mind, typical dosages of sorafenib may range from about 200 mg to about 400 mg. A preferred dose range is on the order of about 100 mg to about 200 mg. In certain embodiments, a patient may receive sorafenib, for example, twice per day orally.

Keeping the above description in mind, typical dosages of advexin (Ad5CMV-p53) may range from about 1 daily intraperitoneal injection for ovarian cancer for 5 days every 3 weeks. Treatment may be repeated every 21 days. For liver cancer, typical dosages of advexin are about 1 percutaneous injection to a maximum of two lesions on day 1. Treatment is repeated every 28 days for up to 6 courses. For breast cancer, typical dosages of advexin (Ad5CMV-p53) are intralesional injection on days 1 and 2. Treatment repeats every 3 weeks for up to 6 courses. For glioma, an initial intratumoral stereotactic injection of adenovirus p53 (Ad-p53) over 10 minutes on day 1. Patients then undergo tumor resection and receive a series of 1-minute injections of Ad-p53 into the resected tumor cavity wall on day 4. In certain embodiments, advexin is administrated together with or after chemotherapeutic agents or radiation therapy.

Keeping the above description in mind, typical dosages of Genentech—Compound 8/cIAP-XIAP inhibitor (Zobel et al., 2006) may range from about 50 mg to about 400 mg. A preferred dose range is on the order of about 100 mg to about 200 mg. In certain embodiments, a patient may receive, for example 8/cIAP-XIAP inhibitor, once per day intravenously.

Keeping the above description in mind, typical dosages of Abbott Laboratories—Compound 11 (Oost et al., 2004) may range from about 50 mg to about 400 mg. A preferred dose range is on the order of about 100 mg to about 200 mg. In certain embodiments, a patient may receive, for example Compound 11, once per day intravenously.

Keeping the above description in mind, typical starting dosages of epirubicin may range from about 100 to 120 mg/m² through intravenous infusion. A preferred dose range is on the order of about 75 mg to about 100 mg. In certain embodiments, a patient may receive epirubicin, for example, administered intravenously on Day 1 and repeated every 21 days for 6 cycles.

Keeping the above description in mind, typical dosages of oxaliplatin may range from about 50 mg-200 mg/per treatment through intravenous infusion. A preferred dose range is on the order of about 75 mg to about 150 mg. In certain embodiments, a patient may receive oxaliplatin, for example, administered in combination with 5-FU/LV every 2 weeks. For adjuvant use, treatment is recommended for a total of 6 months (12 cycles. A typical treatment regiment is the following: Day 1, oxaliplatin 85 mg/m² IV infusion in 250-500 mL 5% Dextrose injection, USP (D5W) and leucovorin 200 mg/m² IV infusion in D5W both given over 120 minutes at the same time in separate bags using a Y-line, followed by 5-FU 400 mg/m² IV bolus given over 2-4 minutes, followed by 5-FU 600 mg/m² IV infusion in 500 mL D5W (recommended) as a 22-hour continuous infusion. Day 2, Leucovorin 200 mg/m² IV infusion over 120 minutes, followed by 5-FU 400 mg/m² IV bolus given over 2-4 minutes, followed by 5-FU 600 mg/m² W infusion in 500 mL D5W (recommended) as a 22-hour continuous infusion.

Keeping the above description in mind, typical dosages of methotrexate may range from about 15 to 30 mg daily administered orally or intramuscularly for a five-day course. Such courses are usually repeated for 3 to 5 times as required. A preferred dose range is on the order of about 20 mg. In certain embodiments, a patient may receive methotrexate in combination with other anticancer agents.

Keeping the above description in mind, typical dosages of vinblastine is the following: initiate therapy for adults by administering a single intravenous dose of 3.7 mg/m² of body surface area (bsa). A simplified and conservative incremental approach to dosage at weekly intervals for adults may be outlined as follows: First dose at 3.7 mg/m² bsa, second dose at 5.5 mg/m² bsa, third dose at 7.4 mg/m² bsa, fourth dose at 9.25 mg/m² bsa, and fifth dose at 11.1 mg/m² bsa. The above-mentioned increases may be used until a maximum dose not exceeding 18.5 mg/m2 bsa for adults is reached. It is recommended that the drug be given no more frequently than once every 7 days.

Keeping the above description in mind, vincristine is administered intravenously once a week. The typical starting dosages of vincristine for pediatric patients is 1.5-2 mg/m² and for adults is 1.4 mg/m².

Keeping the above description in mind, typical starting dosages of satraplatin may range from about 100 to 120 mg/m² once daily for 5 consecutive days every 5 weeks. A preferred dose range is on the order of about 80 mg/m².

Keeping the above description in mind, typical starting dosages of ixabepilone may range about 40 mg/m² over 3 h every 3 wk through intravenous infusion. Patients with body surface area more than 2.2 m² should be calculated based on 2.2 m². Ixabepilone may be used in combination with capecitabine.

Keeping the above description in mind, typical dosages of gemcitabine may range about 1000 mg/m² over 30 minutes intravenous infusion on Days 1 and 8 of each 21-day cycle. In certain embodiments, gemcitabine may be used in combination with paclitaxel (breast cancer) and cisplatin (lung cancer).

Keeping the above description in mind, typical dosages of gemcitabine may range about 1000 mg/m² over 30 minutes intravenous infusion on Days 1 and 8 of each 21-day cycle. In certain embodiments, gemcitabine may be used in combination with paclitaxel (breast cancer) and cisplatin (lung cancer). Keeping the above description in mind, typical dosages of doxorubicin when used as a single agent is 60 to 75 mg/m² as a single intravenous injection administered at 21-day intervals. The lower dosage should be given to patients with inadequate marrow reserves due to old age, or prior therapy, or neoplastic marrow infiltration. In certain embodiments, doxorubicin may be used concurrently with other approved chemotherapeutic agents. When used in combination with other chemotherapy drugs, the most commonly used dosage of doxorubicin is 40 to 60 mg/m2 given as a single intravenous injection every 21 to 28 days.

Keeping the above description in mind, typical dosages of DOXIL (doxorubicin HCl liposome injection) should be administered intravenously at a dose of 30-50 mg/m² at an initial rate of 1 mg/min to minimize the risk of infusion reactions. For patients With Multiple Myeloma, Bortezomib is first administered at a dose of 1.3 mg/m² as intravenous bolus on days 1, 4, 8 and 11, every three weeks. DOXIL 30 mg/m² should be administered as a 1-hr intravenous infusion on day 4 following bortezomib.

Keeping the above description in mind, typical dosages of etoposide (ETOPOPHOS) should be administered intravenously at a dose of ranges from 35 mg/m²/day for 4 days to 50 mg/m²/day for 5 days. In certain embodiments, etoposide may be used in combination with other anticancer agents.

Keeping the above description in mind, typical dosages of melphalan (ALKERAN Tablets) should be administered orally at a dose about 6 mg (3 tablets) daily. After 2 to 3 weeks of treatment, the drug should be discontinued for up to 4 weeks. In certain embodiments, melphalan may be used in combination with other anticancer agents including bortezomib.

Keeping the above description in mind, hexamethylamine (Hexylen, Altretamine, Hexastat) as HEXALEN® capsules is administered orally. Doses are calculated on the basis of body surface area. HEXALEN® capsules may be administered either for 14 or 21 consecutive days in a 28 day cycle at a dose of 260 mg/m²/day. The total daily dose should be given as 4 divided oral doses after meals and at bedtime. HEXALEN® capsules should be temporarily discontinued (for 14 days or longer) and subsequently restarted at 200 mg/m²/day.

Keeping the above description in mind, irinotecan (CAMPTOSAR) may be used either as a single agent or in combination with fluorouracil and leucovorin at a dosage of 125 mg/m² intravenously over 90 minutes once a week for four doses or as a single agent at a dosage of 350 mg/m² intravenously over 90 minutes every three weeks, or in combination with fluorouracil and leucovorin at a dosage of 180 mg/m² intravenously over 90 minutes every other week for three doses.

Keeping the above description in mind, typical dosages of PF-04217903 may range from about 50 mg to about 1000 mg administrating orally twice a day. A treatment cycle is considered to be 21 days. A preferred dose range is on the order of about 100 mg to about 500 mg.

Keeping the above description in mind, typical dosages of AMG 208 may range from about 10 mg to about 1000 mg administrating orally twice a day. A preferred dose range is on the order of about 100 mg to about 500 mg.

Keeping the above description in mind, typical dosages of JNJ-38877605 may range from about 10 mg to about 1000 mg administrating orally once or twice a day. A treatment cycle is considered to be 21 days. A preferred dose range is on the order of about 100 mg to about 500 mg.

Keeping the above description in mind, typical dosages of MGCD-265 may range from about 24 mg/m2 to about 340 mg/m2 administrating orally and daily with 7 days on/7 days off schedule for a 28-day cycle. A preferred dose range is on the order of about 200 mg to about 500 mg.

Keeping the above description in mind, typical dosages of SGX-523 may range from about 10 mg to about 500 mg administrating orally twice a day on a 14 days on/7 days off therapy schedule, cycling every 21 days. A preferred dose range is on the order of about 100 mg to about 200 mg.

Keeping the above description in mind, typical dosages of GSK1363089 may range at about 240 mg/d on day 1-5 repeated every 14 days with 5 day on/9 day off schedule or at about 80 mg/d daily. The drug will be administrated orally. A preferred dose range is on the order of about 80 mg to about 200 mg.

Keeping the above description in mind, typical dosages of vandetanib may range from about 100 mg to about 500 mg administrating orally once a day. A preferred dose range is on the order of about 100 mg to about 300 mg.

Keeping the above description in mind, typical dosages of BIBF1120 may range from about 100 mg to about 250 mg administrating orally twice a day in a 20-day continuous dosing regimen. A preferred dose range is on the order of about 100 mg to about 200 mg.

Keeping the above description in mind, the recommended dose of VOTRIENT (pazopanib) may range from about 200 mg to about 800 mg orally once daily without food (at least 1 hour before or 2 hours after a meal).

Keeping the above description in mind, typical dosages of bevacizumab may range from about 5 mg-10 mg/kg every 2 weeks; 5 mg/kg or 10 mg/kg every 2 weeks when used in combination with intravenous 5-FU-based chemotherapy; about 15 mg/kg every 3 weeks in combination with carboplatin and paclitaxel; about 10 mg/kg every 2 weeks in combination with interferon alfa; and about 10 mg/kg every 2 weeks in combination with paclitaxel. Bevacizumab should be administrated through intravenous (IV) infusion over 90 minutes in a 20-day continuous dosing regimen. A preferred dose range is on the order of about 100 mg to about 200 mg.

Keeping the above description in mind, typical dosages of vatalanib may range from about 250 mg to about 2000 mg administrating orally daily in a 28-day continuous dosing regimen. A preferred dose range is on the order of about 1000 mg to about 1500 mg.

Keeping the above description in mind, typical dosages of axitinib may range from about 5 mg to about 30 mg twice daily administrating orally daily. A preferred dose range is on the order of about 5 mg to about 10 mg.

Keeping the above description in mind, typical dosages of E7080 may range from about 0.1 mg-12 mg administrating orally continually twice daily for 2-6 cycles of a 28-day cycle. A preferred dose range is on the order of about 5 mg to about 10 mg.

Keeping the above description in mind, typical dosages of perifosine may range from about 100-600 mg/week administrating orally. A preferred dose range is on the order of about 200 mg to about 400 mg.

Keeping the above description in mind, typical dosages of MK-2206 may range from about 30 mg-60 mg administrating orally every other day in a 28-day cycle. A preferred dose range is on the order of about 30 mg to about 50 mg.

Keeping the above description in mind, typical dosages of temsirolimus may range from about 25 mg-about 50 mg administrating through infused over a 30-60 minute period once a week. A preferred dose range is on the order of about 30 mg.

Keeping the above description in mind, typical dosages of rapamycin may range from about 10 mg-40 mg administrating orally daily. A preferred dose range is on the order of about 20 mg to about 30 mg.

Keeping the above description in mind, typical dosages of BEZ235 may range from about 10 mg-45 mg administrating orally once daily on days 1-28 of the first course. Courses will repeat every 28 days. A preferred dose range is on the order of about 20 mg to about 30 mg.

Keeping the above description in mind, typical dosages of GDC-0941 may range from about 60 mg-80 mg administrating orally once daily or twice a day. A preferred dose range is on the order of about 40 mg to about 50 mg.

Keeping the above description in mind, typical dosages of PLX-4032 may range from about 200 mg-960 mg administrating orally twice daily. A preferred dose range is on the order of about 300 mg to about 500 mg.

Keeping the above description in mind, typical dosages of imatinib may range from about 400 mg-800 mg administrating orally daily or twice daily. A preferred dose range is on the order of about 400 mg to about 500 mg.

Keeping the above description in mind, typical dosages of AZD0530 may range from about 100 mg-500 mg/week administrating orally. A preferred dose range is on the order of about 100 mg to about 250 mg.

Keeping the above description in mind, typical dosages of VELCADE (bortezomib) is 1.3 mg/m2 administered as a 3 to 5 second bolus IV injection in combination with oral melphalan and oral prednisone for nine 6-week treatment cycles. In cycles 1 through 4, bortezomib is administered twice weekly (days 1, 4, 8, 11, 22, 25, 29 and 32). In cycles 5 through 9, bortezomib is administered once weekly (days 1, 8, 22 and 29). At least 72 hours should elapse between consecutive doses of bortezomib.

Keeping the above description in mind, typical dosages of XAV-939 may range from about 100 mg-500 mg/week administrating orally. A preferred dose range is on the order of about 100 mg to about 250 mg.

Keeping the above description in mind, the dosage of the chemotherapeutic agent or cytotoxic drug may be less than that normally used when administered in combination with the CD44-Fc fusion protein, as described herein these fusion proteins sensitizes cancer cells to cytotoxic drugs.

### EXAMPLES

The present invention is described further below in working examples which are intended to further describe the invention without limiting the scope therein.

### Materials and Methods

In the examples below, the following materials and methods were used.

### Patient Glioma Samples

The glioma tissues were obtained from Cooperative Human Tissue Network (CHTN) at University of Pennsylvania and The Ohio State University. Human tissues were used in accordance with the approved Human tissue study protocol.

### Expression Data Mining

The Oncomine database (www.oncomine.org, Compendia Bioscience, Ann Arbor, Mich.) was searched for CD44 mRNA expression levels in human glioma tissues and other human cancer types compared to their normal counterparts.

Expression Profiling and Real-Time Quantitative PCR (qPCR)

To compare gene expression profiles, human U133v2 gene chips (Affymetrix) were used and the probes derived from three independently transduced and pooled puromycin-resistant U87MG or WM793 cells that re-express merlin (U87MG/Wm793merlin) or were transduced with empty retroviruses (U87MG/WM793 wt) following standard protocols.

### Cell Lines and Reagents

Human glioma cells, U138MG, LN118, LN229, and A172 cells (ATCC); SNB19, SNB75, SNB78, U118MG, U87MG, U251, U373MG, SF763, SF767, SF268, SF539, SF188, SF295, and SF242 (UCSF and NCI), and normal human astrocytes (NHAs, ALLCELLS, Inc) were maintained according to the providers' and manufacturers' instructions. Anti-MST1/2, -Lats1/2 (Bethyl Lab), -CD44, -Erk1/2, -AKT, -JNK, -p21, -p38, -p53, -cIAP1/2, and -merlin (Santa Cruz), -actin (Sigma), -nestin (Millipore), -sox-2 (R&D systems), -v5 epitope, -phospho-merlin, -puma (Invitrogen), -cleaved caspase 3, -phospho-Erk1/2, -phospho-AKT, -phospho-JNK, -phospho-p38, -phospho-MST1/2, -phospho-Lats1, -phospho-YAP (Cell signaling), -YAP, -phospho-Lats2 (Abnova) and -heparan sulfate (HS, CalBiochem) antibodies were used in the experiments. Apoptag kit was from Chemicon and anti-Brdu from Roche.

### Establishment of Primary Human Glioma, Lung, Breast, and Ovarian Cancer Spheres

Fresh human glioblastoma, lung cancer, prostate cancer, breast cancer, ovarian cancer, and melanoma tissues were obtained from Cooperative Human Tissue Network (CHTN) at University of Pennsylvania and The Ohio State University. The tissues were dissociated into single cells by 0.4% collagenase type I (Sigma C0130) and plated in ultra-low attachment plates in serum-free cancer stem cell culture medium, which is DMEM/F12 supplemented with B27 (Invitrogen), EGF (10 ng/mL, BD Biosciences), and FGF-2 (20 ng/mL, BD Biosciences). After formation of the initial spheres, cancer spheres, including glioma spheres, were passaged approximately every-two week by dissociating the spheres with 0.05% trypsin-ethylenediamine tetraacetic acid (EDTA).

### Engineering CD44-Fc Fusion Expression Vectors and Knockdown Constructions

Human total spleen RNAs were obtained from Clontech. Total RNAs from human skin tissues (CHTN-University of Pennsylvania) and T47D human breast cancer cells (ATCC) were isolated using RNeasy Mini Kit (Qiagen) according to the manufacturer's instructions. cDNA was synthesized from 5 μg of total RNA using Superscript II RNase H⁻ reverse transcriptase (Invitrogen). Human Fc fragment was obtained by PCR using human spleen cDNAs as templates, Pfu DNA polymerase (Stratagene), and a pair of primers as the following: forward primer, 5′-gacaaaactcacacatgcccaccg-3′ (SEQ ID NO. 71) and reverse primer, 5′ tcatttacccggagacagggagag-3′ (SEQ ID NO. 72). Human skin and T47D human breast cancer cells expressing many CD44 isoforms including human CD44v3-v10, CD44v8-v10, and CD44s were obtained. Human soluble CD44 isoforms were obtained by PCR using mixture of human skin and T47D cDNAs as templates, Pfu DNA polymerase (Stratagene), a pair of primers as the following: forward primer, 5′-acc atg gac aag ttt tgg tgg cac-3′ (SEQ ID NO. 73) and reverse primer, 5′-ttctggaatttggggtgtccttat-3′ (SEQ ID NO. 74). All the resulting PCR products were cloned into pEF6/v5-HisTOPO expression vectors (Invitrogen). The clones with correct human Fc fragment and soluble CD44 were identified. These fragments were then subcloned into the retroviral expression vector pQCXIP (BD Bioscience) to generate human soluble CD44-Fc (hsCD44) fusion expression constructs. The soluble human CD44v3-v10, v8-v10, or soluble CD44s were fused in frame to the human Fc fragment using a MfeI restriction site (CAATTG). Retroviruses were generated using these expression constructs and pVSVG/GP2 in 293 cells following the manufacturer's instructions (BD). All expression constructs were verified by DNA sequencing.

### Deletion and Point Mutagenesis

The following soluble human CD44-Fc fusion protein constructs have been generated CD44s-, CD44v3-v10-, CD44v8-v10-, CD44v4-v10-, CD44v6-v10-, CD44v7-v10-, CD44v9-v10-, and CD44v10-Fc. The following soluble human CD44-Fc fusion protein constructs are being generated by deletional mutagenesis: CD44v5-v10-, CD44v9-, CD44v8-, CD44v7-, CD44v6-, CD44v5-, CD44v4-, and CD44v3-Fc. Deletional mutagenesis is performed by using soluble human CD44v3-v10-Fc in the retroviral expression vector pQCXIP (BD Bioscience) as the template together with the ExSite mutagenesis kit (Stratagene), and different pairs of appropriate primers corresponding to the sequences of 24 nucleotides before and after the segments intended to be deleted as described (Bai et al., 2007).

The following soluble human CD44R41A-Fc mutated fusion protein constructs have been generated: CD44sR41A-, CD44v8-v10R41A-, and CD44v3-v10R41A-Fc. The following soluble human CD44R41A-Fc mutated fusion protein constructs will be generated by point mutation: CD44v4-v10R41A-, CD44v5-v10R41A-, CD44v6-v10R41A-, CD44v7-v10R41A-, CD44v9-v10R41A-, CD44v10R41A-, CD44v9R41A-, CD44v8R41A-, CD44v7R41A-, CD44v6R41A-, CD44v5R41A-, CD44v4R41A-, CD44v3R41A-Fc. The point mutation in CD44sR41A-, CD44v8-v10R41A-, and CD44v3-v10R41A-Fc were generated by using soluble human CD44s-, CD44v8-v10-, CD44v3-v10-Fc in retroviral expression vector pQCXIP (BD Bioscience) as the templates together with the QuikChange® II Site-Directed Mutagenesis Kit (Stratagene), and a pairs of appropriate primers: forward, 5′-gtg gag aaa aat ggt gcc tac agc atc tct cgg-3′ (SEQ ID NO. 75) and reverse, 5′-ccg aga gat get gta ggc acc att ttt etc cac-3′ (SEQ ID NO. 76). The retroviruses were generated by using these expression constructs and pVSVG/GP2-293 cells following the manufacturer's instructions (BD Bioscience). Similar procedures will be used to generate additional CD44R41A-Fc constructs.

### Produce and Purify Soluble CD44-Fc and Soluble CD44R41A-Fc Fusion Proteins

Cos-7 cells infected with the retroviruses carrying hsCD44v3-v10-Fc, hsCD44v6-v10-FC, hsCD44v8-v10-Fc, hsCD44s-Fc, hsCD44v3-v10R41A-Fc, hsCD44v8-v10R41A-Fc, and hsCD44sR41A-Fc constructs were cultured in RPMI medium containing 10% fetal bovine serum (FBS) to reach confluence then switched to serum free RPMI medium (SFM) to culture for additional three days. The collected SFM was purified through protein A columns (GE Healthcare Biosciences). Before elution from protein A column, some of preparations of the bound CD44-Fc fusion proteins were treated heparinase I (10 units/ml) and heparinase III (2 unit/ml) or at 37° C. for 4 h.

### Luciferase Reporter Assay

To measure canonical Wnt signaling in U87MGwt and U87MGmerlinS518D, U87MGmerlin, and U87MGmerlinS518A cells, the beta-catenin-responsive luciferase reporter construct (TopFlash, Addgene), which contains TCF/LEF binding sites and a negative control construct, FopFlash, which contains mutated TCF/LEF binding sites, was used. These reporters were transfected transiently into these transduced glioma cells in triplicate. The luciferase activity in these transfected cells were measured 24 hours post-transfection following the manufacturer's instructions (Promega) using a Modulus Microplate Luminometer/Fluorometer (Turner Biosystems).

To knock down human CD44 expression, several shRNAmir (expression Arrest™ microRNA-adapted shRNA) and TRC (the RNAi consortium) constructs against human CD44 and a non-targeting shRNAmir and non-targeting TRC control constructs were obtained from Open Biosystems and Addgene (a non-profit plasmid repository, www.addgene.org). Lentiviruses carrying these shRNAs were generated following the manufacturer's instructions. Expression Arrest™ microRNA-adapted shRNA (shRNAmir) are designed to mimic a natural microRNA primary transcript, enabling specific processing by the endogenous RNAi pathway and producing more effective knockdown. microRNA-30 adapted design contains mir-30 loop and context sequences (Silva et al., 2005)

TABLE 2
Sequence Listings for the CD44 Antisense Constructs

| nucleotides | Sequence | SEQ ID No. |
|---|---|---|
| shRNA-TRC- | sense loop antisense: | 31 |
| CD44#1 | GCCCTATTAGTGATTTCCAAA CTCGAG | |
| | TTTGGAAATCACTAATAGGGC | |
| shRNA-TRC- | sense loop antisense: | 32 |
| CD44#2 | CGGAAGTGCTACTTCAGACAA CTCGAG | |
| | TTGTCTGAAGTAGCACTTCCG | |
| shRNA-TRC- | sense loop antisense: | 33 |
| CD44#3 | CCTCCCAGTATGACACATATT CTCGAG | |
| | AATATGTGTCATACTGGGAGG | |
| shRNA-TRC- | sense loop antisense: | |
| CD44#4 | CCAACTCTAATGTCAATCGTT CTCGAG | 34 |
| | AACGATTGACATTAGAGTTGG | |
| shRNA-TRC- | sense loop antisense: | |
| CD44#5 | CGCTATGTCCAGAAAGGAGAA CTCGAG | 35 |
| | TTCTCCTTTCTGGACATAGCG | |
| shRNAmir- | mir-30 context sequence sense loop antisense mir-30 | 36 |
| CD44#1 | context sequence: | |
| | TGCTGTTGACAGTGAGCG | |
| | AGGTGTAACACCTACACCATTA | |
| | TAGTGAAGCCACAGATGTA | |
| | TAATGGTGTAGGTGTTACACCC | |
| | TGCCTACTGCCTCGGA | |
| shRNAmir- | mir-30 context sense loop antisense mir-30 context: | 37 |
| CD44#2 | TGCTGTTGACAGTGAGCG | |
| | ACGCAGATCGATTTGAATATAA | |
| | TAGTGAAGCCACAGATGTA | |
| | TTATATTCAAATCGATCTGCGC | |
| | TGCCTACTGCCTCGGA | |
| shRNAmir- | mir-30 context sense loop antisense mir-30 context: | 38 |
| CD44#3 | TGCTGTTGACAGTGAGCG | |
| | CCCTCCCAGTATGACACATATT | |
| | TAGTGAAGCCACAGATGTA | |
| | AATATGTGTCATACTGGGAGGT | |
| | TGCCTACTGCCTCGGA | |
| shRNA-TRC-NT | CCGCAGGTATGCACGCGT (Addgene) | 39 |
| shRNAmir-NT | mir-30 context sense loop antisense mir-30 context: | 40 |
| | TGCTGTTGACAGTGAGCG | |
| | ACCTCCACCCTCACTCTGCCAT | |
| | TAGTGAAGCCACAGATGTA | |
| | ATGGCAGAGTGAGGGTGGAGGG | |
| | TGCCTACTGCCTCGGA | |

Lenti- and Retroviral Transduction

U87MG and U251 human glioma cells were seeded in 6-well plates and allowed to grow for overnight. The subconfluence U87MG, and U251 cells were first transduced with the retroviruses carrying luciferase with a hygromycin-resistant gene, and then transduced with the retroviruses carrying the empty retroviral expression vector or human soluble (hs) CD44-Fc fusion constructs with a puromycin-resistant gene. The pooled populations of drug resistant cells were expanded, and portions of the cells were used to assess their expression of the transduced gene products. Anti-CD44 and anti-human IgG antibodies were used to detect the expression level of hsCD44-Fc fusion proteins.

CD44 knockdown was accomplished using lentiviruses carrying shRNAs against human CD44 or non-targeting control shRNAs following the manufacturer's instructions. Infected cells were selected for their resistance to hygromycin and puromycin. The pooled populations of the drug resistant cells were expanded and portions of the cells were used to assess the expression level of endogenous CD44. Anti-CD44 antibodies (Santa Cruz) were used for assessing endogenous level of CD44.

### Glioma Sphere Transduction

Human glioma spheres (HGSs) were disaggregated with 0.05% trypsin-ethylenediamine tetraacetic acid (EDTA, Cellgro®) and seeded on the BD BioCoat™ Matrigel™ Matrix 6-well plates, which are designed to maintain and propagate embryonic stem cells in the absence of feeder layers. These cells were transduced with lentiviruses carrying shRNAs against human CD44. After selection with puromycin, the pooled populations of drug-resistant cells were suspended into single cells and cultured in serum-free cancer stem cell culture medium (DMEM/F12 supplemented with B27 (Invitrogen), EGF (10 ng/mL, BD Biosciences), and FGF-2 (20 ng/mL, BD Biosciences)) in ultra-low attachment plates to re-form spheres.

### Western Blot Analysis of CD44 Expression

Cells were extracted with either RIPA buffer (50 mM Tris-HCl (pH7.4) containing 150 mM NaCl, 5 mM EDTA, 1% Triton, 0.1% SDS, 2 mM PMSF, 2 μg/ml leupeptin, and 0.05 U/ml aprotinin) or with 4×SDS Laemmli sample buffer without the dye and protein concentrations were determined using Bio-Rad Dc Protein Assay Reagents. 50-100 μg of extracted proteins were separated by 10% SDS-PAGE. Following electrophoresis, the gels were blotted onto Hybond-ECL membranes (Amersham, Arlington Heights, Ill.). Anti-CD44 antibody (Santa Cruz) was employed to detect CD44.

### Immunocytochemistry of CD44 Expression

Glioma cells with or without CD44 knockdown were cultured in 35 mm dishes in the presence of 10% FBS RPMI for 24 hours. The cells were fixed in 3.7% paraformaldehyde, washed with PBS, and blocked with 2% non-fat milk. Anti-CD44 antibody (Santa Cruz) and FITC-conjugated anti-mouse secondary antibody (Sigma) were employed to detect cell surface CD44.

### Fluorescein-Labeled HA (FL-HA) Binding Assay

FL-HA binding assay was performed as described previously (Xu and Yu, 2003; Yu and Stamenkovic, 1999). Briefly, a total of 5×10⁵ of the transduced glioma cells were seeded onto 35-mm dishes in the presence of PRMI/10% FBS and puromycin. On the following day, the culture medium was replaced by fresh RPMI/10% FBS containing 20 μg/ml Fl-HA. Twenty-four later, the cells were washed extensively with PBS, fixed in 4% paraformaldehyde, washed, mounted, and observed under a fluorescence microscope.

### Subcutaneous Tumor Growth Experiments

Mice were used in accordance with the approved IACUC Protocol. Pooled populations of transduced U87MG and U251 glioma cells were used for subcutaneous tumor growth experiments. 2 or 5×10⁶ glioma cells or 5×10⁶ of glioma cells were injected subcutaneously into each immuno-compromised B6.129S7-Rag1^{tmMom} (Rag1, Jackson Lab) mouse. Six mice were used for each type of the infected glioma. After solid tumors became visible (10-15 days after the injection), the longest and shortest diameters of the solid tumors were measured using a digital caliper every third day for five to seven weeks for gliomas. Tumor volumes were calculated using the following formula: tumor volume=½×(shortest diameter)²×longest diameter (mm³). At end of the experiments, tumors were fixed and sectioned for histological and immunohistochemical analyses.

### Intracranial Tumor Growth Experiment

Mice were used in accordance with the approved IACUC Protocol. Pooled populations of the transduced U87MG and U251 cells were used for the intracranial tumor growth experiments. U87MG (4×10⁵ cells in 10 μl HBSS/Rag1 mouse)/U251 cells (2×10⁵ cells in HBSS/Rag1 mouse) were injected at the bregma 2 mm to the right of the sagittal suture and 3 mm below the surface of the skull. Following injection, mice were closely monitored and the duration of their survival was recorded. Mice that showed signs of distress and morbidity were euthanized and considered as if they had died on that day. Number of surviving mice was recorded. The survival rates were calculated as follows: survival rate (%)=(number of mice still alive/total number of experimental mice)×100%. Mice that were free of symptoms 40 or 60 days after intracranial injection were euthanized and the tissues examined.

### Bioluminescence Imaging Analysis of the Intracranial Gliomas

To monitor the growth of intracranial gliomas in live animal, bioluminescence-imaging approach was used. U87MG and U251 cells were infected with a retroviral-based luciferase expression vector that contains an internal ribosome entry site (IRES) and hygromycin resistance gene. Hygromycin-resistant U87MG-Luc and U251-Luc cells express high levels of luciferase. These cells were then infected with lentiviruses carrying non-targeting shRNAs or shRNAs against human CD44. These double drug resistant cells were injected intracranially into Rag-1 mice at the bregma 2 mm to the right of the sagittal suture and 3 mm below the surface of the skull. 3, 6, 9, 13, 17 days after the injections, bioluminescence images of the intracranial tumors were acquired 12 min after injection of D-luciferin using the same intensity scaling by using IVIS-200 imaging system (Xenogen) at the In Vivo Molecular Imaging Shared Facility at Mount Sinai School of Medicine.

### Histology and Immunohistochemistry

To determine the glioma cell proliferation rate in vivo, 5-Bromo-2′-deoxy-uridine (BrdU) was injected intraperitoneally (i.p.) into mice four hours prior to euthanasia. Tumors including gliomas from the experimental animals were dissected and fixed in formalin (Fisher), washed with PBS, dehydrated through 30%, 70%, 95%, and 100% ethanol and xylene, and embedded in paraffin wax (Fisher). 5-10 μm sections were cut, mounted onto slides and stained with hematoxylin and eosin (Fisher) for histologic analysis. The sections were incubated with anti-BrdU or anti-Ki67 antibodies to detect proliferating cells or with the Apoptag kit to detect apoptotic cells in situ (Lau et al. 2008).

### Western Blot Analysis of Signaling Pathway Proteins

U87MG-NT cells (U87MG cells infected with a mixture lentiviruses carrying non-targeting TRC-NT and shRNAmir-NT constructs) and U87MGshRNA-CD44 cells (U87MG cells infected with a mixture lentiviruses carrying shRNAs against human CD44, TRC-CD44#3 and shRNAmir-CD44#1) were treated with vehicle, 60 μm H₂O₂ or 40 μg/ml TMZ for 30 min, 2 h, 24 h, 48 h, and 72 h. The cells were lysed using 4×SDS Laemmli sample buffer without the dye. Protein concentrations were determined using Bio-Rad Dc Protein Assay Reagents. 100 μg of total protein was loaded in each lane. Actin was included as an internal control for protein loading. The antibodies used against the different signaling mediators are indicated in the figures.

Western blots were also performed using cell lysates derived from U87MG-TN and U87MGshRNA-CD44 cells treated with different growth factors. 2×10⁵ of the glioma cells were seeded into 6-well plates for 24 hours and switched to serum free medium and cultured for additional 72 hours. The serum starved U87MG cells were treated with or without FBS, NGF (10 ng/ml), EGF (2 ng/ml), HB-EGF (5 ng/ml), betacellulin (BTC, 5 ng/ml), epiregulin (Epr, 5 ng/ml), amphiregulin (AR, 5 ng/ml), or HGF (20 ng/ml) for 12 h. The cells were lysed using 4×SDS Laemmli sample buffer without the dye and protein concentrations were determined using Bio-Rad Dc Protein Assay Reagents. 100 μg of total protein were loaded in each lane. Actin was included as an internal control for protein loading. The antibodies used against the different signaling mediators are indicated in the figures.

### Administration of Oxidative Stress

H₂O₂ was added into serum-free glioma culture medium (RPMI) to reach a final concentration of 60 μM. The glioma cells were cultured in the presence of 60 μm H₂O₂ for 30 min, 2 h, 24 h, 48 h, and 72 h.

### Administration of Chemotherapeutic Agents

TMZ was added into the serum-free glioma culture medium (RPMI) to reach a final concentration of 40 μg/ml. The glioma cells were cultured in the presence of 40 μg/ml TMZ for 30 min, 2 h, 24 h, 48 h, and 72 h.

### Methods of Detecting HA Using Biotin-Labeled CD44-Fc Fusion Proteins and Methods of Diagnosing Cancers by Detecting HA

Purified CD44-Fc fusion proteins (hsCD44s-Fc, hsCD44v8-v10-Fc, and hsCD44v3-v10-Fc) were labeled with biotin using EZ-Link Biotinylation Kits (Thermo Scientific) following the manufacturer's instruction. Human tumor paraffin sections were deparaffinized and rehydrated. After blocking with 2% BSA, the sections were incubated with biotinylated CD44-Fc fusion proteins (1 μg/ml) for overnight at 4 degree. biotinylated CD44-Fc fusion proteins were detected by VECTASTAIN ABC kit.

To detect plasma HA level, at least 200 μl blood from each transgenic mice (MMTV-PyVT and MMTV-ActErbb2, Jackson Lab) bearing breast cancer, Rag-1 mice bearing gliomas derived from MSSM-GBMCSC-1 or Glioma 261 cells, or control health mice were collected. Blood samples from six mouse of each type of mice were collected and plasma samples were generated immediately. 50 μl plasma from each sample was loaded in triplicate into each well of an Elisa plate that has been pre-coated with CD44-Fc fusion proteins. The CD44-Fc bound HA was detected by biotinylated CD44-Fc fusion proteins and AP-conjugated avidin. The developed color was measure by an Elisa machine at 405 nm.

### Prostate, Colon, Breast, Lung, Ovarian, Liver, Pancreatic, and Head-Neck Cancer Models and Melanoma Model

PC3/M human prostate cancer cells, HCT116 and KM20L2 human colon cancer cells, MX-2 and SW613 human breast carcinoma cells, NCI-H125 human non-small cell lung cancer cells, NCIH460, human large cell lung cancer cells, and OVCAR-3 human ovarian cancer cells were transduced with luciferases and shRNAs against human CD44 or control non-targeting shRNAs and selected for their resistance to hygromycin and puromycin. M14 human melanoma cells, SCC-4 human head-neck carcinoma cells, BXPC-3 human pancreatic cancer cells, and SK-Hep-1 human liver cancer cells were transduced with retroviruses carrying CD44s-Fc, CD44v3-v10-Fc, and CD44v8-v10-Fc constructs or empty expression vector. Pooled populations of the drug-resistant cancer cells were used for subcutaneous tumor growth experiments. 5×10⁶ of these cancer cells were injected subcutaneously into each immuno-compromised B6.129S7-Rag1^{tmMom} (Rag1, Jackson Lab) mice. Six mice were used for each type of the infected cancer cells. The longest and shortest diameters of the solid tumors were measured using a digital caliper at the end of the experiments. Tumor volumes were calculated using the following formula: tumor volume=½×(shortest diameter)²×longest diameter (mm³). At end of the experiments, tumors were fixed and sectioned for histological and immunohistochemical analyses.

### Additional Cancer Models

Xenograft and orthotopical mesothelioma tumor models in Rag-1 mice: 5×10⁶ of human malignant mesothelioma cells, H-MESO-1, H-MESO-1A, and/or MSTO-211H (ATCC and NCI-DCTD Tumor/Cell line repository in Frederick) will be injected subcutaneously and orthotopically into the right pleural cavity immunocompromised Rag 1 mice.

Xenograft melanoma models: 5×10⁶ of human melanoma cells, MEWO, SKMEL5, SKMEL2, and/or A375 (ATCC and NCI-DCTD Tumor/Cell line repository in Frederick), will be injected subcutaneously into immunocompromised Rag 1 mice.

Xenograft sarcoma models: 5×10⁶ of human sarcoma cells, SKN-MC and A673 cell (ATCC), will be injected subcutaneously into immunocompromised Rag 1 mice.

Xenograft pancreatic cancer models: 5×10⁶ of human pancreatic cancer cells, Pane-1, HPAC, MIA PaCa-2, and/or AsPC-1 pancreatic cancer cells, will be injected subcutaneously into immunocompromised Rag 1 mice.

Xenograft hepatoma models: 5×10⁶ of human hepatoma cells, Hep 3B2.1-7 hepatoma cells, will be injected subcutaneously into immunocompromised Rag 1 mice.

Xenograft multiple myeloma models: 5×10⁶ of human multiple myeloma cells, U266 and MC/CAR cells, will be injected subcutaneously into immunocompromised Rag 1 mice.

Ascites ovarian cancer model in Rag-1 mice: 5×10⁶ of human SKOV3ip and OVCAR-3ip human ovarian cancer cells will be injected into Rag-1 mice intraperitoneally (ip).

Xenograft and/or bone metastatic prostate cancer models: 5×10⁶ of human prostate cancer cells, 22Rv1, will be injected into Rag-1 mice subcutaneously or intracardiacally into each Rag-1 mice, respectively.

Xenograft and/or metastatic lung cancer models: 5×10⁶ of human lung cancer cells, A549 and LX529 will be injected into Rag-1 mice subcutaneously or intravenously into Rag-1 mice, respectively.

Xenograft and orthotopical breast cancer models: 5×10⁶ of human breast cancer cells, MX-2 and SW613, will be injected subcutaneously or into Rag-1 mouse mammary fat pad, respectively.

Cancer stem cell models: Fresh human glioblastoma, human melanoma, lung, breast, prostate, ovarian, head-neck, kidney, and colon cancer tissues were obtained from Cooperative Human Tissue Network (CHTN) at University of Pennsylvania and The Ohio State University. The tissues were dissociated into single cells by 0.4% collagenase type I (Sigma C0130) and plated in ultra-low attachment plates in serum-free cancer stem cell culture medium, which is DMEM/F12 supplemented with B27 (Invitrogen), EGF (10 ng/mL, BD Biosciences), and FGF-2 (20 ng/mL, BD Biosciences). After formation of the initial spheres, the tumor spheres were passaged approximately every week by dissociating the spheres with 0.05% trypsin-ethylenediamine tetraacetic acid (EDTA). The tumor spheres were implanted subcutaneously into Rag-1 mice.

Statistics

One-way ANOVA statistic analyses were performed to analyze statistical differences of the tumor volumes and growth rates between the control and experimental groups. LogRank analyses were performed for the survival experiments. Differences were considered statistically significant at p<0.05.

### Example 1

### CD44 is Upregulated in Human Glioblastoma Multiforme (GBM)

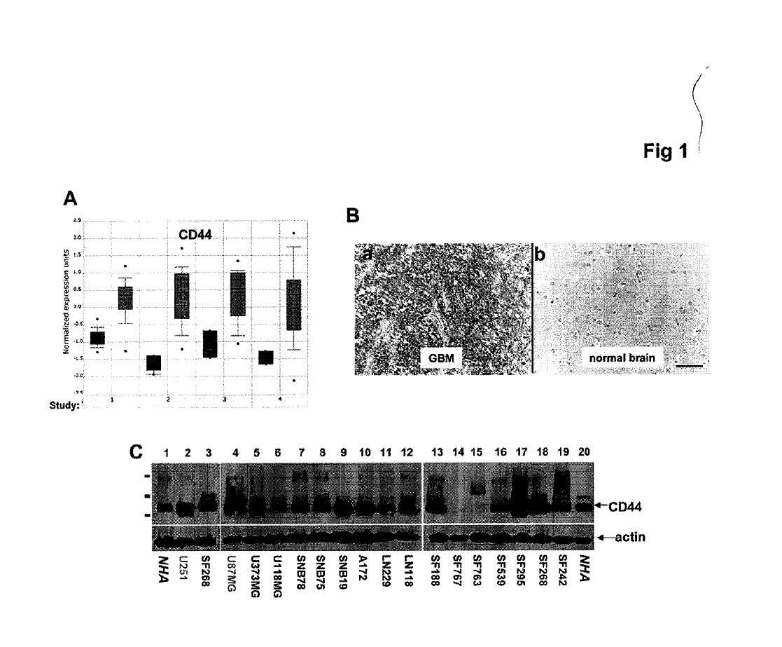

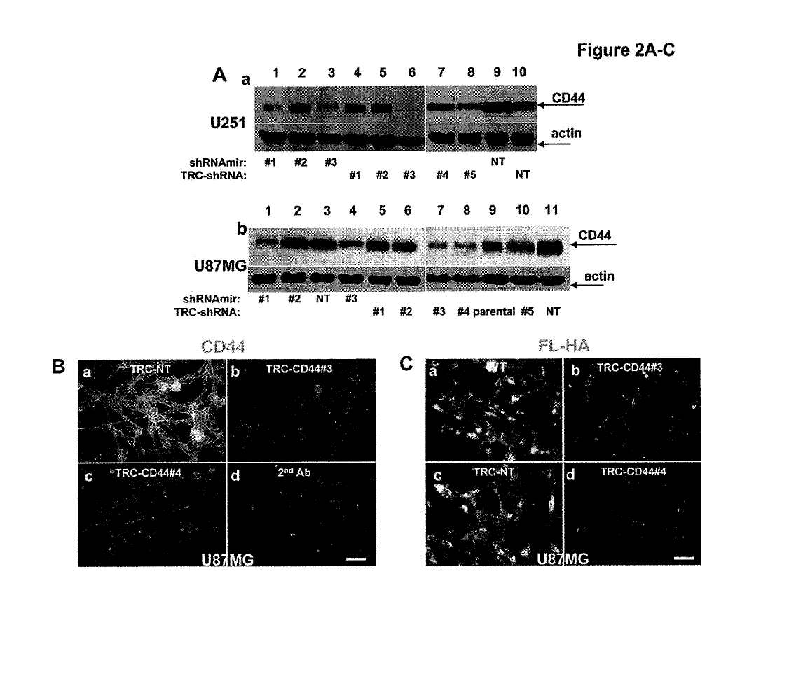

To determine the expression level of CD44 in GBM, available gene expression datasets at www.oncomine.org were mined. In four independent datasets, CD44 transcripts were consistently upregulated in human GBM compared to either normal brain (FIG. 1A, studies 1, 2, and 4) (Bredel et al., 2005; Liang et al., 2005; Sun et al., 2006) or normal white matter (FIG. 1A, study 3) (Shai et al., 2003). Immunohistochemistry of paraffin sections of primary tumors showed that CD44 is upregulated in all 14 GBM cases analyzed compared to eight cases of normal human brain (FIG. 1B).

To address the role of CD44 in glioma growth and progression, expression levels of the CD44 protein in a variety of human glioma cell lines were analyzed. Human glioma cell lines were derived from ATCC, UCSF, and NCI-DCTD Tumor/Cell line repository in Frederick. The majority of human glioma cells tested express higher levels of CD44 than normal human astrocytes (NHAs) and the standard 85-90 kDa form (CD44s, FIG. 1C) was the predominant isoform expressed. Based on their high CD44 expression level and their tumorigenicity in immunocompromised mice, U87MG and U251 human glioma cells were selected to investigate the role of CD44 in glioma growth and progression and the mechanisms whereby CD44 may contribute to the processes.

### Example 2

### Lentiviral Based shRNAs Effectively Knocked Down CD44 Expression in Human Glioblastoma Multiforme (GSM) Cells

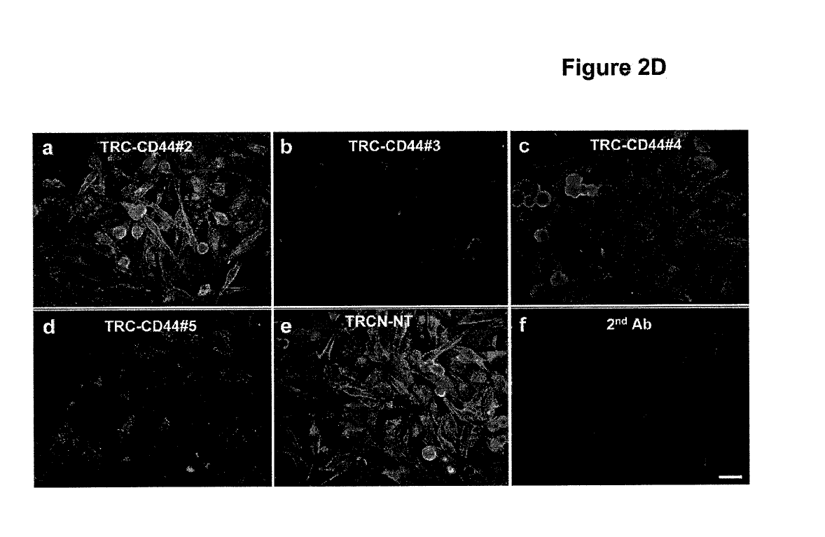

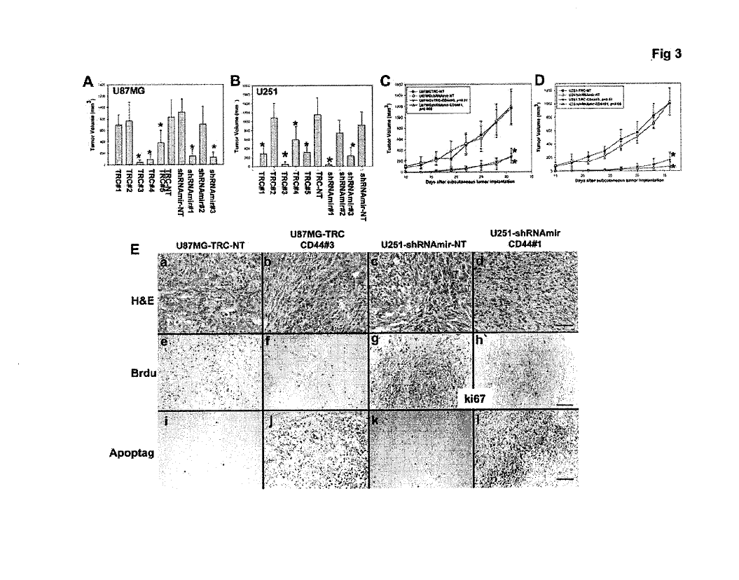

To knock down endogenous CD44 expression effectively in U251 and U87MG cells, a set of human CD44-specific TRC-shRNA (shRNA-TRC-CD44#1-#5) and shRNAmir (shRNAmir-CD44#1-#3) constructs (Open Biosystems) were screened. Non-targeting control shRNAs (shRNA-TRC-NT and shRNAmir-NT) were included in the screen as negative controls. These shRNA vectors were lentiviral-based and contained the internal ribosome entry site (IRES)/GFP and/or puromycin-resistance gene located at the 3′-termini of the shRNA inserts. The IRES element in the shRNAmir construct ensures that all the puromycin-resistant cells express the inserted shRNAs and allows use of the GFP expression level as an indicator of the shRNA expression efficiency. Lentiviruses containing these shRNA constructs were used to infect U87MG-Luc and U251-Luc cells that had been transduced with and expressed luciferase. Luciferase activity allowed efficient monitoring of intracranial growth of these cells (Lau et al., 2008). After selection of the infected cells with puromycin, expression levels of endogenous CD44 were assessed in pooled populations of puromycin-resistant GBM cells. Two out of three shRNAmir constructs (shRNAmir-CD44#1 and shRNAmir-CD44#3) and 1-2 TRC-shRNA (shRNA-TRC-CD44#3 and/or shRNA-TRC-CD44#4) knocked down CD44 expression efficiently in these two glioma cell lines, as assessed by real-time qPCRs (data not shown) and Western blot analysis (FIG. 2A) and immunocytochemistry (FIG. 2B, D). Other CD44-specific shRNAs reduced CD44 expression in variable degrees whereas the non-targeting controls displayed no effect. Because CD44 is a major cell surface receptor of HA, the capacity of CD44-depleted cells to bind fluorescein-labeled HA (FL-HA) was assessed. Effective knockdown of CD44 expression dramatically reduced the ability of glioma cells to bind and endocytose FL-HA, whereas non-targeting shRNAs had no effect (FIG. 2C, and data not shown).

### Example 3

### Depletion of CD44 Expression Inhibited Subcutaneous Growth of U87MG and U251 Cells by Inhibiting their Proliferation and Promoting Apoptosis In Vivo

Pooled populations of the transduced U87MG and U251 cells that displayed different degrees of CD44 depletion were first used in subcutaneous (s.c.) tumor growth experiments to determine how reduced CD44 expression affects glioma growth in vivo. Reduced CD44 expression in these cells correlated with reduced tumor volumes 5 weeks following injection of the GBM cells (FIG. 3A-B). Growth curves of tumors derived from the glioma cells infected with control non-targeting shRNAs, shRNA-TRC-NT and shRNAmir-NT, or two CD44-specific shRNAs that effectively knock down CD44 expression, shRNATRC-CD44#3 and shRNAmirCD44#1, further demonstrated that CD44 depletion significantly inhibited subcutaneous glioma growth (FIG. 3C-D). To begin to address the mechanisms underlying the growth inhibitory effect of CD44 knockdown, proliferation and survival of the transduced U87MG and U251 cells in situ were analyzed. shRNAs that knock down CD44 expression, but not the control non-targeting shRNAs, inhibited glioma cell proliferation (FIG. 3E-e-h) and promoted apoptosis in vivo (FIG. 3E-i-l).

### Example 4

### Knockdown of CD44 Expression Inhibited Intracranial Growth of U87MG and U251 Gliomas

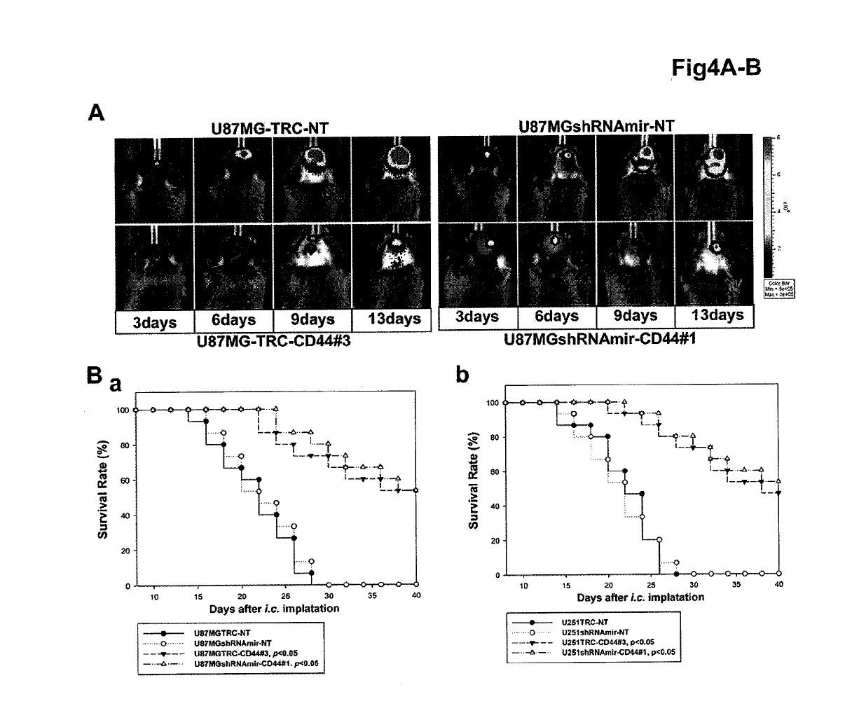

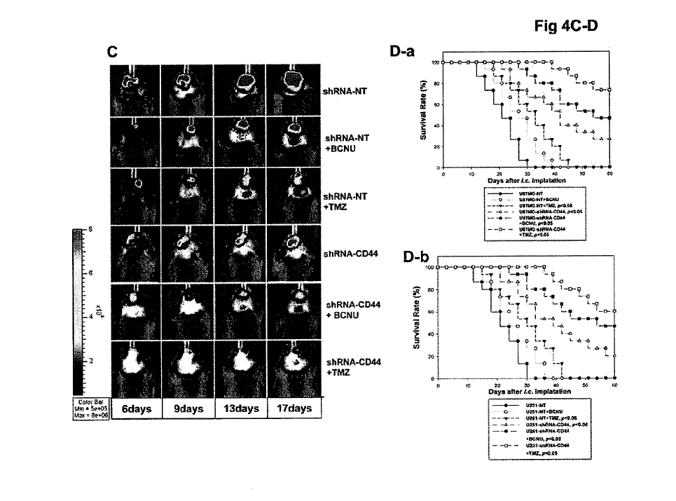

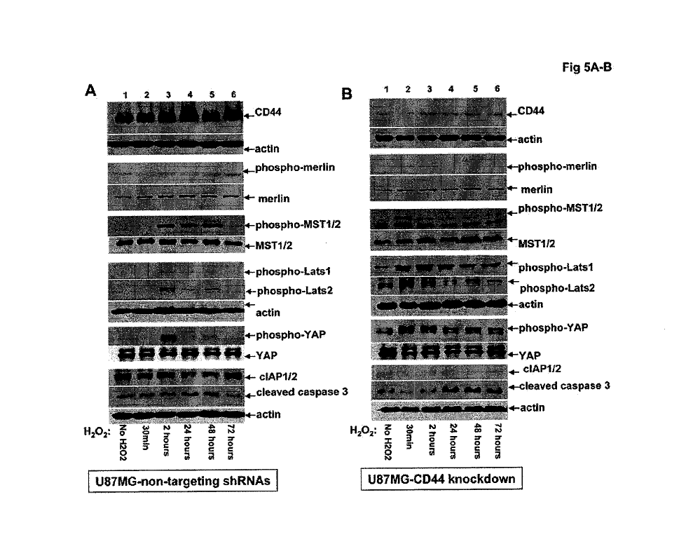

To determine the effect of CD44 knockdown on intracranial glioma growth, double drug-resistant pooled populations of glioma cells that express high levels of luciferase and display significant CD44 depletion were injected intracranially into immunocompromised Rag-1 mice. Three, six, nine, and thirteen days after injection, bioluminescence images of the intracranial tumors were acquired using an IVIS-200 imaging system (Xenogen, FIG. 4A and data not shown). The mice were closely monitored for the duration of their survival as defined in Materials and Methods. Suppression of CD44 expression significantly inhibited intracranial tumor growth and increased the survival time of the experimental animals compared to mice injected with U87MG/U251-Luc cells transduced with non-targeting shRNAs cells (FIG. 4B).

To confirm the effect of reduced CD44 expression on intracranial glioma growth, an inducible CD44 knockdown system was established in U87MG-luc and U251-Luc cells by using two TRIPZ lentiviral Tet-On shRNAmir constructs (Open Biosystems), which contain two of the effective shRNAs against CD44 (shRMAmir #1 and #3, FIG. 2 and data not shown). These TRIPZ constructs expressed shRNAs in the presence of doxycycline (Dox) and effectively knocked down CD44 expression in U87MG and U251 cells, whereas control non-targeting TRIPZ shRNA had no effect on CD44 expression (data not shown). Immunocompromised Rag-1 mice were fed regular or doxycycline-impregnated (625 ppm; Harlan-Teklad) food pellets for three days prior of intracranial injection of glioma cells. The experimental mice were continuously fed with regular or doxycycline-impregnated food pellets throughout the experiments. Inducible knockdown of CD44 inhibited intracranial glioma growth and prolonged mouse survival (data not shown), supporting initial observations.

### Example 5

### Reduced CD44 Expression Sensitizes Glioma Cells to Cytotoxic Drugs In Vivo

The first-line cytotoxic drugs for GBM are temozolomide (TMZ) and carmustine (BCNU). Based on previous observations that CD44 provides essential survival signals to metastatic breast cancer cells (Yu et al., 1997), the possibility that reduced CD44 expression may inhibit survival signaling and sensitize glioma cells to BCNU and TMZ treatment in vivo was addressed. Mice were injected intracranially with U87MG-Luc and U251-Luc cells, depleted or not of endogenous CD44, and treated sequentially with a single dose of BCNU (10 mg/kg, iv) or TMZ (5 mg/kg, ip). BCNU and TMZ displayed a weak and a moderate inhibitory effect on glioma growth, respectively, when used as single agents (FIG. 4C-D). CD44 depletion, however, sensitized the response of glioma cells to BCNU and TMZ, as demonstrated by the observation that the combination of CD44 knockdown and treatment with BCNU or TMZ resulted in a synergistic inhibition of intracranial glioma formation as determined by markedly prolonged the median survival length of the mice (FIG. 4D).

### Example 6

### CD44 Attenuated Activation of the Mammalian Equivalent of Hippo Signaling Pathway and Played a Key Role in Regulating Stress and Apoptotic Responses of Human GBM Cells

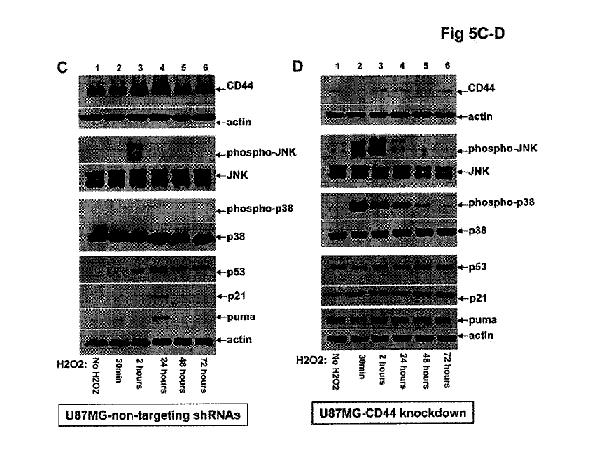

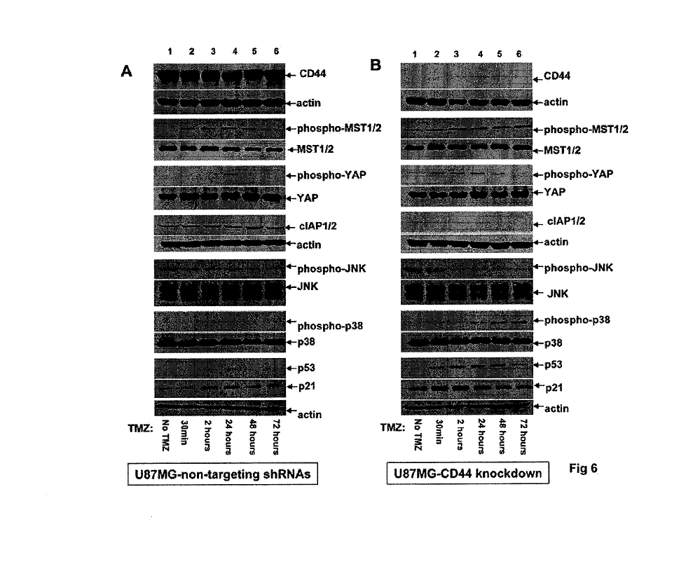

Radiation therapy provides another option for GBM patients. Radiation therapy and some cytotoxic agents generate reactive oxygen species (ROS), which constitute a major inducer of cell death resulting in their anti-glioma effects. To address the molecular mechanisms that underlie the observed chemosensitizing effect of CD44 knockdown on glioma cells, how reduced CD44 expression affects GBM cell response to oxidative stress induced by H₂O₂ and cytotoxic stress induced by TMZ was investigated. U87MG cells transduced with a mixture of viruses carrying the control non-targeting shRNAmir-NT and TRC-NT or with a mixture of two of most effective shRNAs against CD44 (shRNAmirCD44#1 and TRC-CD44#3, FIG. 2) were used in these experiments. Reduced expression of endogenous CD44 in human GBM cells resulted in the enhanced and sustained response of the cells to oxidative and cytotoxic stresses and reduced viability of these cells (FIG. 5 and data not shown).

MST1/2 plays an important role in mediating oxidative-stress-induced apoptosis (Lehtinen et al., 2006), and we have shown that MST1/2 functions downstream of merlin in human GBM cells (Lau et al., 2008). Compared to the GBM cells expressing a high level of endogenous CD44, the cells with depleted endogenous CD44 responded to oxidative stress with robust and sustained phosphorylation/activation of MST1/2 and Lats1/2, phosphorylation/inactivation of YAP, and reduced expression of cIAP1/2 (FIG. 5A-B). These effects correlate with reduced phosphorylation/inactivation of merlin, increased levels of cleaved caspase-3 and reduced cell viability (FIGS. 5B and 3E, and data not shown). By contrast, a higher level of endogenous CD44 promotes phosphorylation/inactivation of merlin, inhibits the stress induced activation of entire mammalian equivalent of Hippo signaling pathway and up-regulated cIAP1/2, leading to inhibition of caspase-3 cleavage and apoptosis (FIG. 5A, 3E, data not shown). Together, these results place CD44 upstream of the mammalian Hippo signaling pathway (merlin-MST1/2-Lats1/2-YAP-cIAP1/2) and suggest a functional role for CD44 in attenuating tumor cell responses to stress and stress-induced apoptosis.

Because MST1/2 kinases have multiple downstream effectors and are implicated in several signaling pathways, whether known effectors of MST1/2 also function downstream of this newly established CD44-MST1/2 signaling axis was investigated. These results indicate that knockdown of CD44 results in elevated and sustained activation of JNK and p38 stress kinases in glioma cells exposed to oxidative stress (FIG. 5D). In addition, oxidative stress induced sustained up-regulation of p53, a known downstream effector of JNK/p38, and its target genes p21 and puma in CD44-deficient glioma cells (FIG. 5D), whereas the GBM cells with high levels of endogenous CD44 attenuated activation of JNK/p38, and inhibited induction of p53, p21, and puma (FIG. 5C).

Caspase-3 cleavage is an indicator of cellular apoptosis. The in vitro data using H₂O₂ treatment demonstrates caspases-3 cleavage (FIG. 5 B), suggesting that the combination of the reduced expression of endogenous CD44 in human GBM cells with oxidative stress would result in a decrease in GBM tumor size.

Although H₂O₂ was not administered in vivo, chemotherapy and radiation therapy act to generate H₂O₂. FIG. 4 (C-D) demonstrates that the combination of a reduction in the expression of endogenous CD44 in human GBM cells coupled with chemotherapeutic agents results in a decrease in tumor size and an increase in survival time. Therefore, it would be expected that a reduction in the expression of endogenous CD44 in human GBM cells coupled with radiation therapy would act to decrease the GBM tumor size as well as increase the survival time as both types of therapies would result in the production of H₂O₂.

To address the mechanism whereby CD44 depletion sensitizes glioma cells to cytotoxic drugs in vivo (FIG. 4C-D), similar experiments were performed to those outlined in FIG. 5 but using TMZ instead of H₂O₂ to induce cytotoxic stress in the glioma cells with high or low CD44 expression. Similar to their response to oxidative stress, glioma cells expressing a very low level of CD44 mounted robust and sustained activation of MST1/2 upon exposure to TMZ, along with phosphorylation/inactivation of YAP that correlated with reduced levels of cIAPs, activation of p38 but not JNK, and up-regulation p53 and its target gene p21 (FIG. 6). Together, these results establish a novel role of CD44 in inhibiting stress/apoptotic responses of tumor cells by attenuating activation of the mammalian Hippo signaling pathway and provide a first molecular explanation for how up-regulation of CD44 may constitute a key event in tumor cell resistance to stress of a broad range of origins, including that generated by host defense and therapeutic intervention.

### Example 7

### CD44 Modulated ErbB and c-Met Receptor Tyrosine Kinase (RTK) Mediated Growth-Signaling Pathways in Glioma Cells

In vivo results show that CD44 knockdown inhibits proliferation of the GBM cells in vivo (FIG. 3E). Previous studies have shown that CD44 is a co-stimulator of ErbB and c-Met RTK signaling pathways (Orian-Rousseau et al., 2002; Toole, 2004; van der Voort et al., 1999), which may account for the reduced in vivo proliferation of CD44-depleted glioma cells, given that RTK signaling pathways are strongly implicated in glioma progression. To determine whether knockdown of CD44 diminishes EGF family ligand- and HGF-induced activation of the downstream signaling pathways, serum starved CD44-high or -low U87MG cells were treated with different RTK ligands, including EGF family ligands, heparin-binding EGF (HB-EGF), betacellulin (BTC), amphiregulin (AR) and epiregulin (Epr)), HGF, NGF, and 10% fetal bovine serum (FBS). Reduced expression of CD44 diminished EGF family ligand- and HGF- but not NGF- and FBS-induced phosphorylation of Erk1/2 kinase but not that of AKT kinase (FIG. 7), suggesting that CD44 preferentially modulates proliferation but not survival signaling pathways activated by these growth factors and that CD44 regulates survival signaling pathway through the Hippo pathway.

### Example 8

### Transcript Profiling of U87MGmerlin and WM793merlin Cells Suggests that Merlin, a Downstream CD44 Effector that is Negatively Regulated by CD44, is a Mediator of a Master Regulator of Several Important Signaling Pathways

CD44 and merlin negatively regulate each other function (Bai et al., 2007 and Xu et al., 2010). U87MG cells responded to the growth inhibitory effect of merlin in a dramatic fashion (Lau et al., 2008), suggesting that downstream signaling pathways of merlin are intact in these cells even though merlin expression is down regulated and CD44 expression is up-regulated. This cell model results in an excellent opportunity to identify the differentially expressed genes and the altered signaling pathways in response to merlin re-expression. These differentially expressed gene may represent the essential downstream effectors of merlin and CD44, which are likely either hyperactive or hypoactive when merlin function is lost or impaired and CD44 is up-regulated in human gliomas. Deregulation of these signaling pathways may lead to gliomagenesis and/or devastating progression of this disease.

To identify downstream effectors that mediate the potent anti-glioma effect of merlin, gene expression profiles of three independently-transduced and pooled U87MGₘₑᵣₗᵢₙ and U87MG_{wt} cells, which express high and low level of merlin, respectively, were compared using human U133v2 gene chips (Affymetrix). The microarray results indicated that the expression of merlin in U87MGₘₑᵣₗᵢₙ cells is ˜three fold higher than in U87MG_{wt} cells. 362 genes whose expression increased and 364 genes whose expression decreased in U87MGₘₑᵣₗᵢₙ cells compared to U87MG_{wt} cells were identified. They can be categorized into the genes involved in adhesion, migration, organization of actin-cytoskeleton, cell cycle, survival, and signal transduction. These genes were imported to David Functional Annotation Bioinformatics Microarray Analysis software (http://david.abcc.ncifcrf.gov/home.jsp, NIAID/NIH) to enrich for functionally related gene groups. After classification of these transcripts into functional pathways, we found that merlin re-expression results in increased expression of transcripts that activates Hippo signaling pathway as well as increased expression of molecules that inhibit Wnt signaling pathway and decreased expression of transcripts that activate Wnt and HGF/c-Met and pleiotrophin (PTN)/Anaplastic lymphoma kinase (ALK) signaling pathways (FIG. 8).

To establish the common changes in the expression profiles induced by merlin among different tumor types, the effect of merlin on human melanoma growth was investigated. It was determined that merlin expression is down-regulated in human melanoma cell lines and that increased expression of wt merlin significantly inhibits subcutaneous growth of WM793 human melanoma cells in vivo (data not shown). Further assessment of the transcript profiles of WM793_{wt} and WM793ₘₑᵣₗᵢₙ cells demonstrated that increased expression of merlin significantly up-regulates 697 genes, many of which display anti-tumor properties, and down-regulates 736 genes, many of which display pro-tumor activity (data not shown). These significantly up- and down-regulated genes were imported to David Functional Annotation Bioinformatics Microarray Analysis software to enrich functional-related genes and generate the signaling pathways that are significantly affected by increased expression of merlin. These outputted data were then compared with that derived from U87MG glioma cells and the common alterations induced by merlin were identified. Together, these data indicated that increased expression of merlin activates Hippo and inhibits Wnt and c-Met signaling pathways (FIG. 8).

### Example 9

### Merlin Inhibits Wnt-Signaling

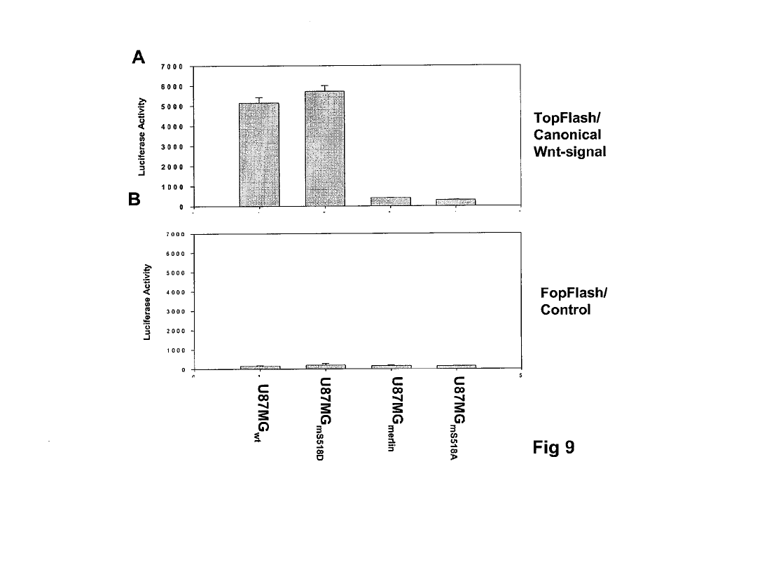

These merlin-induced changes of expression were then investigated at the functional level. Since canonical Wnt signaling regulates gene expression by modulating the levels of beta-catenin expression, a co-activator of the T-cell factor/lymphocyte enhancer factor (TCF/LEF) transcription factors, reporter assays using a beta-catenin-responsive luciferase reporter construct, TopFlash (Addgene), were performed. FopFlash, which contains mutated TCF/LEF binding sites, was used as a negative control. It was found that beta-catenin transcriptional activity is inhibited by wild-type merlin and merlinS518A, but not by merlinS518D (FIG. 9) (Lau et al., 2008).

### Example 10

### CD44 and Merlin-Mediated Signaling Events and their Potential Cross-Talk

A working model of CD44 and merlin-mediated signaling events and their potential cross-talk (the components of Drosophila Hippo signaling pathway are underlined): merlin functions upstream of the mammalian Hippo (merlin-MST1/2-LATS1/2-YAP) and JNK/p38 signaling pathways and plays an essential role in regulating the cell response to the stresses and stress-induced apoptosis as well as to proliferation/survival signals. Merlin antagonizes CD44 function and inhibits activities of RTKs and the RTK-derived growth and survival signals. CD44 function upstream of mammalian Hippo signaling pathway and enhances activities of RTKs

### Example 11

### Antagonists of CD44, hsCD44-Fc Fusion Proteins, Serve as Effective Therapeutic Agents Against Human GBM in Mouse Models

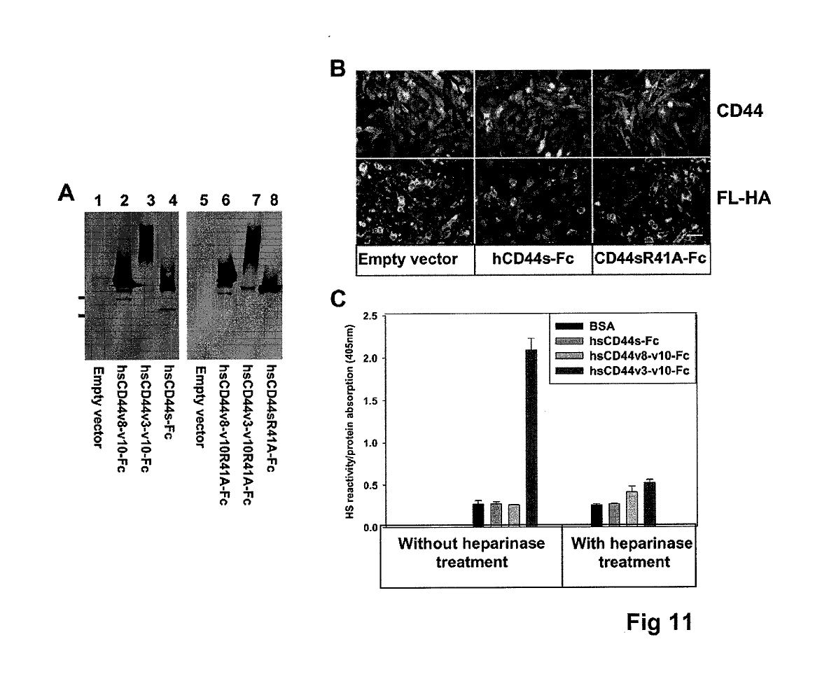

To determine whether antagonists of CD44 can be used to inhibit glioma progression in preclinical mouse models, several fusion proteins composed of the constant region of human IgG1 (Fc) (Holash et al., 2002; Kim et al., 2002; Sy et al., 1992) fused to the extracellular domain of CD44v3-v10, CD44v8-v10, CD44s, CD44v3-v10R41A, CD44v8-v10R41A, or CD44sR41A were developed (FIG. 11). The antibody-like characteristics of these fusion proteins provide them with favorable pharmacokinetics and biodistribution profile in vivo in addition to relative ease of production and purification in vitro. Receptor-Fc fusion proteins may function by trapping ligands and/or by interfering with endogenous receptor functions.

Mutating R41 to A abolishes the ability of CD44 to bind to HA. The ability of the mutated CD44 to bind to all other ligands and CD44 sheddases, however, will likely be preserved, which is important because ligands other than HA and the CD44 sheddase are likely to be very important to exert the pro-tumor activity of CD44. While the loss of HA binding may reduce some activity of CD44R41A-Fc against certain cancers, this modification may improve the biodistribution and bioavailability.

The v3 exon of CD44 contains a Ser-Gly-Ser-Gly motif for covalent attachment of heparan sulfate (HS) side chains (Bennett et al., 1995). To assess whether hsCD44v3-v10-Fc proteins are modified by HS, purified hsCD44s-Fc, hsCD44v8-v10-Fc, and hsCD44v3-v10-Fc fusion proteins were treated with or without heparinase I/III before elution from protein A columns. These proteins were then coated on Elisa plates in triplicate. After blocking with BSA, the coated proteins were tested for reactivity with anti-HS antibody. The intensity of the reaction, as assessed by a colorimetric assay, was normalized by the reactivity with anti-CD44 antibody, which provides relative quantity of the coated fusion proteins on the plates. These results showed that only hsCD44v3-v10-Fc was modified by HS and stained positively with anti-HS antibody. The observed reactivity was sensitive to heparinase I/III treatment (FIG. 11C).

U87MG and U251 cells were transduced with retroviruses carrying the expression constructs encoding these CD44-Fc and CD44R41A-Fc fusion proteins or empty expression vector. Pooled puromycin resistance cells expressed high levels of hsCD44v3-v10-Fc, hsCD44v8-v10-Fc, hsCD44s-Fc, hsCD44v3-v10R41A-Fc, hsCD44v8-v10R41A-Fc, hsCD44sR41A-Fc fusion proteins (FIG. 11A,FIG. 12A). Whether the soluble CD44-Fc fusion proteins are capable of altering FL-HA binding to endogenous GBM cell surface CD44 was assessed. It was found that expression of the CD44-Fc fusion proteins reduced binding of FL-HA to the GBM cells (FIG. 11B). These cells were then compared to empty vector-transfected cells for subcutaneous and intracranial growth in Rag-1 mice. hsCD44v3-v10-Fc, hsCD44v8-v10-Fc, and hsCD44s-Fc expression markedly inhibited subcutaneous and intracranial growth of U87MG and U251 cells and significantly extended survival of mice bearing the intracranial tumors. The hsCD44v3-v10-Fc fusion protein displayed the most profound inhibitory effect (FIGS. 12B and C).

CD44 has multiple ligands including HA, osteopontin, heparin binding growth factors, fibronectin, serglycin, laminin, MMP-9, and fibrin (Bennett et al., 1995; Ponta et al., 2003; Stamenkovic, 2000; Stamenkovic and Yu, 2009; Toole, 2004) and cooperates with several RTKs and other cell surface receptors (Orian-Rousseau et al., 2002; Stamenkovic, 2000; Stamenkovic and Yu, 2009). Many of CD44 functions are mediated through its interaction with HA (Toole, 2004), which is abolished by the single R41A mutation (Peach et al., 1993). To determine whether the CD44-HA interaction alone is responsible for the GBM promoting activity of CD44, pooled populations of U87MG and U251 cells expressing hsCD44sR41A-Fc or hsCD44v3-v10R41A-Fc were generated and their anti-GBM effects were compared with that of their wild type counterparts. Unlike wild type CD44-Fc fusion proteins, CD44R41A-Fc proteins are incapable of inhibiting FL-HA binding to the GBM cells (FIG. 11B and data not shown). However, whereas hsCD44sR41A-Fc displayed a weak anti-GBM effect, hsCD44v3-v10R41A-Fc retained a substantial level of anti-GBM activity (FIG. 12C-c and data not shown), which is consistent with the finding that hsCD44v3-v10-Fc fusion protein exerts the most potent anti-GBM effect of the three CD44-Fc fusion proteins tested, suggesting a mechanism of action in addition to trapping HA. Together, these results suggest that CD44, and especially CD44 variants, promote tumor progression both in an HA-dependent and HA-independent fashion.

Finally, the anti-GBM efficacy of purified hsCD44s-Fc fusion proteins in pre-established intracranial gliomas resulting from injection of 5×10⁵ U87MG or U251 cells into Rag-1 mice was assessed. Intracranial tumors were grown for 5 days before the mice were treated by intravenous injection of 0.9% NaCl containing 5 mg/kg human IgG or purified hsCD44s-Fc fusion proteins every third day until completion of the experiments. Systemic delivery of hsCD44s-Fc fusion proteins but not human IgG markedly inhibited intracranial growth of U87MG and U251 cells and significantly (p<0.001) extended median survival of the experimental mice (FIG. 13 A-B). GBM and brain issue were collected at the time of mouse euthanasia, sectioned, and stained with anti-human IgG antibody to assess the bio-distribution of hsCD44-Fc fusion proteins. The results showed that hsCD44-Fc fusion proteins readily penetrated tumor blood vessels and displayed a remarkable intra-glioma distribution pattern whereas negligible fusion proteins were observed in normal adjacent brain tissue, most likely due to the presence of an intact blood-brain-barrier (FIG. 13C). These results show that CD44-Fc proteins preferentially accumulate within the tumor tissue, which contains leaky blood vessels. Together, the results demonstrate that hsCD44-Fc fusion proteins are potentially attractive therapeutic agents for GBM. In addition, normal host tissues were stained with H&E to assess potential toxicity of systematical delivery of the fusion proteins. Upon carefully gross and histological examination, no apparent toxicity and necrosis to normal tissues were observed (FIG. 13D).

### Example 12

### Knockdown of CD44 Sensitizes the Responses of Cancer Cells to the erbB and c-Met RTK Inhibitors

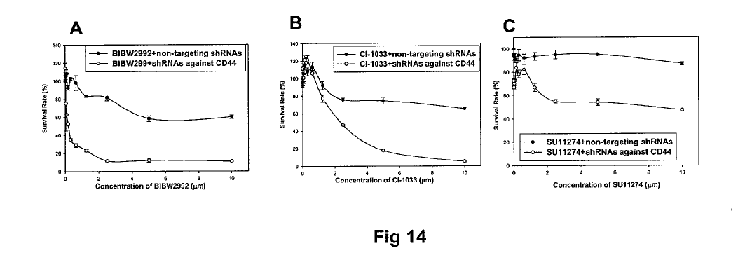

As shown in FIG. 7, CD44 plays an important role in enhancing the growth signals derived from ErbB and c-Met RTKs. To determine whether knockdown of CD44 sensitizes the responses of GBM cells to the pharmacologic inhibitors of erbB and c-Met RTKs, glioma cell viability assays in the presence or absence of different concentrations of inhibitors of erbB and c-Met RTKs, with or without CD44 knockdown, were performed. The results showed that shRNAs knocked down CD44 expression sensitized the response of U87MG cells to a dual inhibitor of EGFR/erbB-2 (BIBW2992), a pan inhibitor of EGFR/erbB2/4 (CI-1033) and a c-Met inhibitor (SU11274; LC Laboratories, Selleck Chemicals Co.) (FIG. 14), providing evidence that targeting CD44 and erbB or c-Met together can achieve synergistic inhibitory effects in the cancer where these molecules play important roles.

### Example 13

### hsCD44-Fc Fusion Proteins Sensitize the Responses of GBM Cells to Chemotherapy and Targeted Therapy

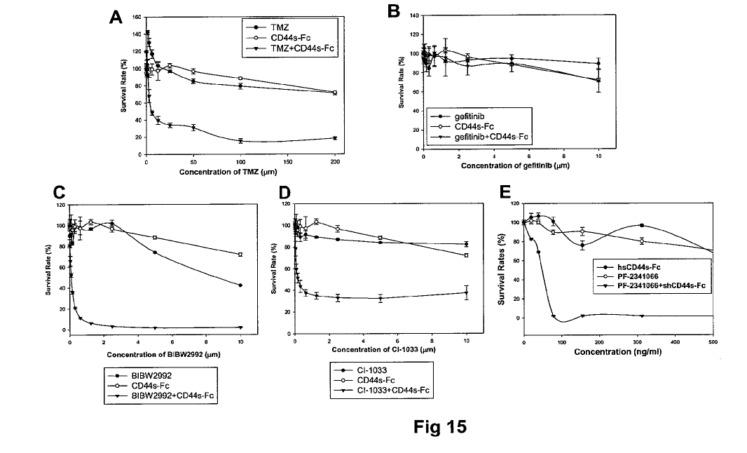

To determine whether CD44 antagonists, hsCD44s-Fc fusion proteins, sensitize the responses of GBM cells to chemotherapeutic agents and pharmacologic inhibitors of erbB and c-Met RTKs, glioma cell viability assays in the presence or absence of different concentrations of TMZ, inhibitors of erbB and c-Met RTKs with or without purified hsCD44s-Fc fusion proteins or human IgG were performed. The results showed that hsCD44s-Fc fusion proteins but not human IgG sensitize the response of U87MG cells to TMZ, a dual inhibitor of EGFR/erbB-2 (BIBW2992), a pan inhibitor of EGFR/erbB2/4 (CI-1033), and a c-Met inhibitor (PF-2341066, Selleck Chemicals Co.) (FIG. 15).

### Example 14

### hsCD44s-Fc Fusion Proteins Display Low Cytotoxicity Towards a Panel of Normal Cells

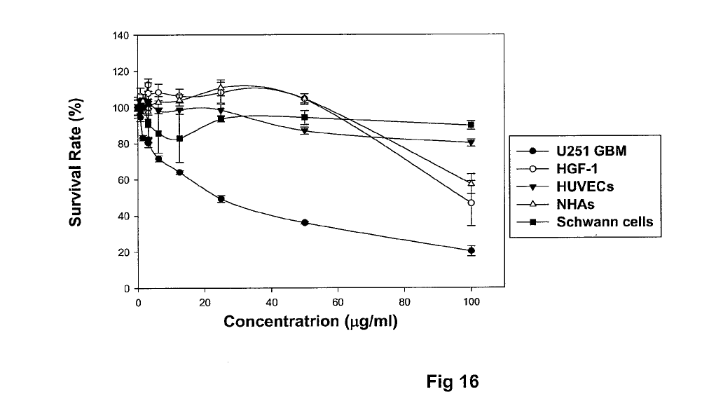

Two important characteristics used to define good cancer therapy targets are high expression of the targets in tumor cells and low or absent expression in normal cells and increased dependency of tumor cells on the target functions. CD44 meets these criteria. To assess potential toxicity of CD44-Fc fusion proteins towards normal cells, cell viability assays using a panel of normal cells in the presence or absence of different amount of purified hsCD44s-Fc fusion proteins were performed. The results demonstrated that CD44-Fc fusion proteins displayed low toxicity towards normal human astrocytes (NHAs), Schwann cells, fibroblasts (HGF-1) and endothelial cells (HUVECs) comparing to U251 GBM cells (FIG. 16).

### Example 15

### CD44 is Required for Self-Renewal and In Vivo Growth of GBMCSCs

Stem cells exhibit the characteristic of self-renewal. To determine the contribution of CD44 to the self-renewal capacity of glioma CSC spheres, primary human glioma spheres (HGSs) from fresh GBM tissues (CHTN) were established. Human GBMCSC spheres, MSSM-GBMCSC-1 and -2, derived from fresh GBM tissues have self-renewal capacity, express stem cell markers (Sox-2 and nestin), and can be readily transduced using retro- and lenti-viruses to express or to knock down expression of the genes of interests (FIG. 17A-C). shRNAs knocked down of CD44 expression in theses GBMCSCs inhibited the sphere formation (FIG. 18), demonstrating that CD44 is important for maintenance glioma stem cells and its targeting helps to eliminate cancer stem cells and stop the recurrence of malignant cancers. In addition, it was found that MSSM-GBMCSC-1 and -2 readily form invasive intracranial tumors in Rag-1 mice (FIG. 17D and data not shown) and overexpression of hsCD44s-Fc fusion proteins inhibits intracranial growth of MSSM-GBMCSC-1 cells (FIG. 17D-b).

### Example 16

### CD44 is Up-Regulated in a Variety of Human Cancer Types

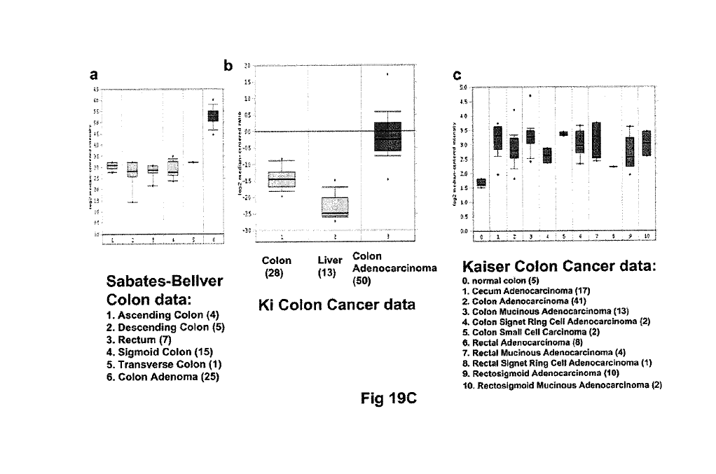

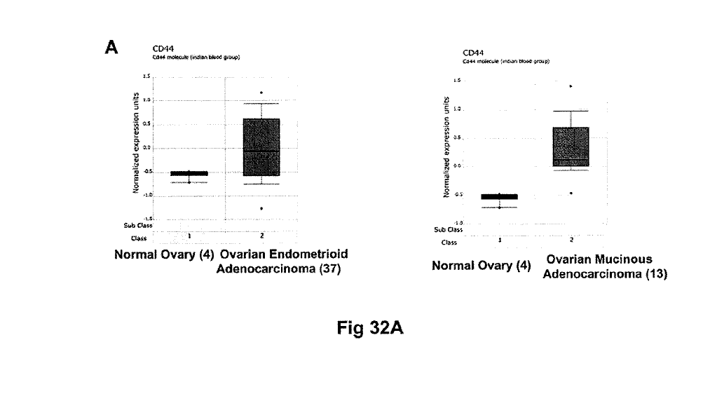

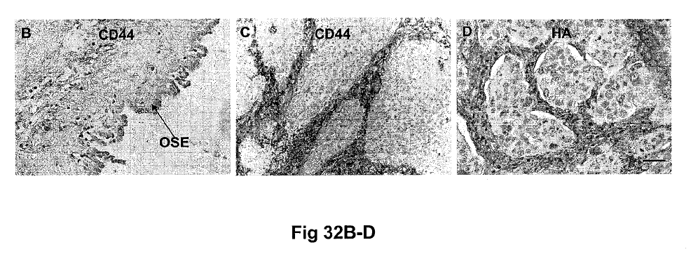

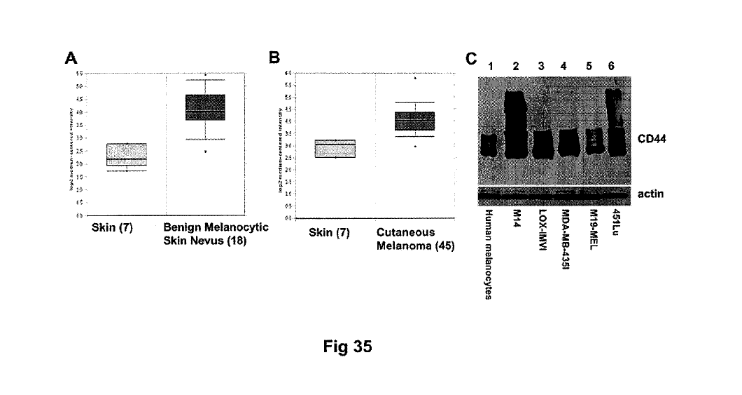

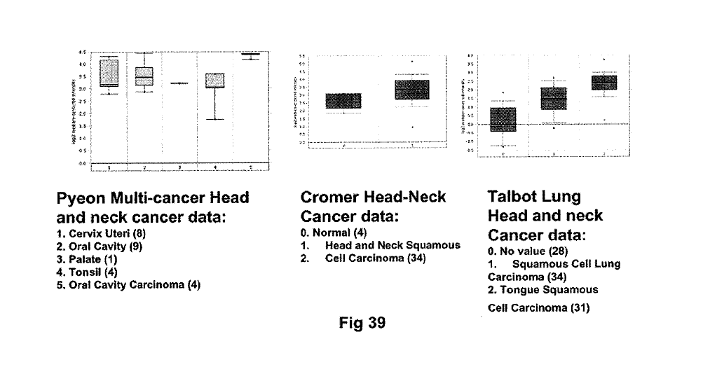

To determine the expression level of CD44 in colon cancer, ovarian cancer, head and neck squamous carcinoma, renal cell carcinoma, melanoma, gastric cancer, and esophageal cancer, available gene expression datasets at www.oncomine.org were mined. We found that CD44 transcripts were up regulated in human colon (FIG. 19A, 19C) (Graudens et al., 2006; Notterman et al., 2001), ovarian (FIG. 32A) (Hendrix et al., 2006), head and neck squamous carcinoma (FIG. 38A, FIG. 39) (Ginos et al., 2004), renal cell carcinoma (FIG. 37, FIG. 38B) (Gumz et al., 2007), melanoma (FIG. 35A-B), gastric cancer (FIG. 42), and esophageal cancer (FIG. 43) compared to their normal counterparts. Data was derived from oncomine (www.oncomine.org).

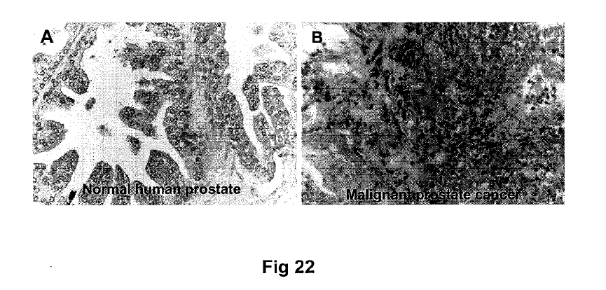

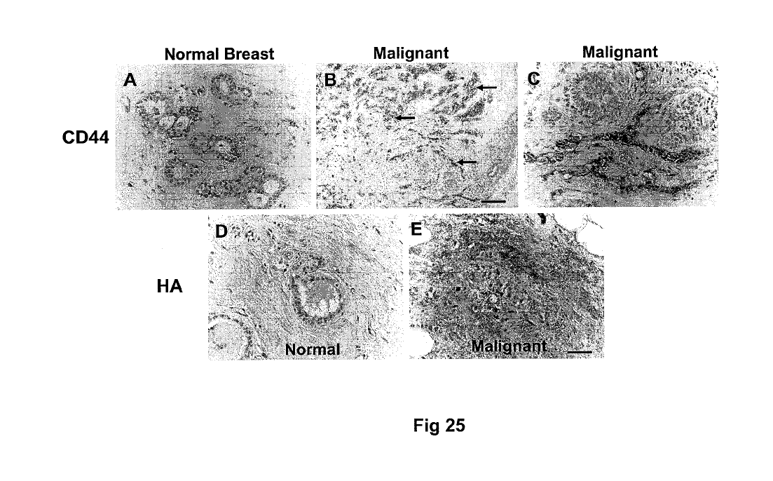

In addition, immunohistochemistry analysis of paraffin sections of primary human tumors showed that CD44 is up regulated in malignant/metastatic colon cancer (FIG. 19B), prostate cancer (FIG. 22), malignant breast cancer (FIG. 25), and metastatic ovarian cancer (FIG. 32B-C) comparing to their normal counterpart tissues or primary tumors.

### Example 17

### Knockdown of CD44 Expression Inhibited the In Vivo Growth of a Variety of Human Cancer Cells

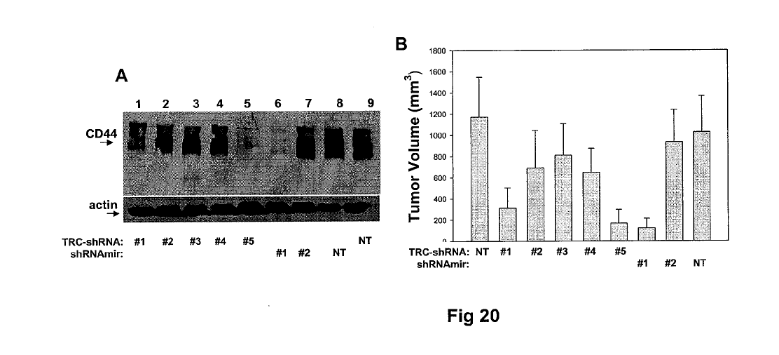

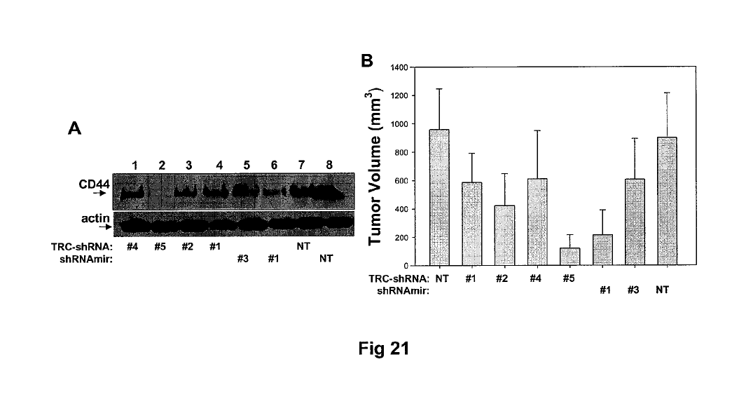

To knock down endogenous CD44 expression in HCT116 and KM20L2 human colon cancer cells, PC3/M human prostate cancer cells, MX-2 and SW613 human breast cancer cells, NCI-H125 and NCI-H460 human lung cancer cells, and OVCAR-3 human ovarian cancer cells, a set of human CD44-specific TRC-shRNA (shRNA-TRC-CD44#1-#5) and shRNAmir (shRNAmir-CD44#1-#3) constructs (Open Biosystems) were screened. Non-targeting control shRNAs (shRNA-TRC-NT and shRNAmir-NT) were included in the screen as negative controls. Lenti-viruses containing these shRNA constructs were used to infect the cancer cells. Following selection of the infected cells with puromycin, the expression level of endogenous CD44 was assessed in pooled populations of puromycin-resistant cancer cells. At least two shRNAs effectively knocked down CD44 expression in these cancer cells as assessed by western blot analysis (FIG. 20A, 21A, 23A, 26A, 27A, 30-31A, and 33A). Other CD44-specific shRNAs reduced CD44 expression in variable degrees, whereas the non-targeting controls displayed no effect. Pooled populations of these transduced cancer cells, displaying different degrees of CD44 depletion, were used in subcutaneous (s.c.) tumor growth experiments to determine how reduced CD44 expression affects their subcutaneous growth in vivo. Results showed that reduced CD44 expression in these cells correlated with reduced tumor volumes 6 weeks following injection of these cells (FIG. 20-21B, 23B, 26-27B, 30-31B, and 33B), establishing that CD44 is required for in vivo growth of these types of cancer cells, and therefore, is a prime target of therapeutic intervention of these cancer types.

### Example 18

### Purified CD44-Fc Fusion Proteins Inhibit In Vivo Growth of Human Prostate Cancer

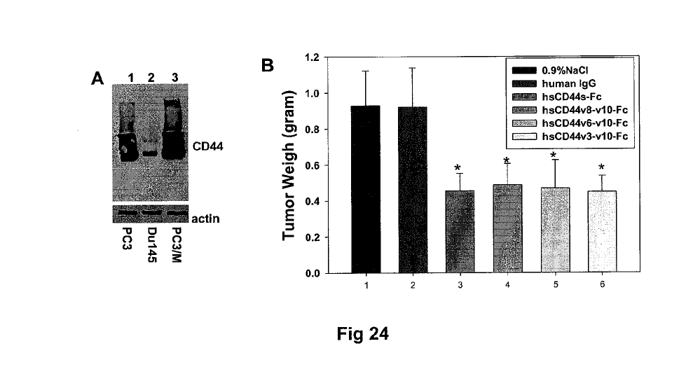

CD44 expression by three human prostate cancer cell lines was assessed. It was found that the most aggressive prostate cell line, PC3/M cell, expresses the highest level of CD44 (FIG. 24A). To assess the effect of purified hsCD44-Fc fusion proteins on PC3/M cell growth in vivo, 5×10⁶ PC3/M cells were injected subcutaneously into each Rag-1 mice. The tumors were allowed to growth for ˜two weeks when the tumor volumes reach ˜150 mm³. The mice bearing similar size tumors were separated into 6 groups (6mice/group) and were treated with 4 intratumoral injections of 5 μl/injection of 10 mg/ml of hsCD44s-Fc, hsCD44v8-v10-Fc, hsCD44v6-v10-Fc, hsCD44v3-v10-Fc, or human IgG, or 0.9% NaCl (FIG. 24B). The experiments were stopped when the tumors of the control groups (treatment of human IgG or 0.9% NaCl) reached 1 cm in their longest diameters. All the tumors were dissected out and weighted. Our results showed that CD44-Fc fusion proteins but not 0.9% NaCl or human IgG significantly inhibited growth of PC3/M cells in vivo (FIG. 24B).

### Example 19

### Human Malignant Breast-Cancer-Cell-Infiltrated Host Stroma Expresses a High Level of CD44 and Invasive Breast Cancer Stroma Accumulates a Higher Level of HA

To determine the role of CD44 in breast cancer progression and in maintenance of breast cancer stem cell (BCSC), CD44 protein and HA levels in human malignant breast cancer tissues (obtained from CHTN—at University of Pennsylvania) were measured. Compared to normal breast tissues (FIG. 25A), CD44 is highly up-regulated in the breast cancer cells infiltrated into host stroma (FIG. 25B-C). Additionally, HA accumulates in malignant breast cancer stroma (FIG. 25E) compared to normal breast stroma (FIG. 25D). HA in the paraffin sections was detected by biotinylated CD44-Fc fusion proteins.

### Example 20

### Establishment of Human BCSCs and In Vivo Breast Cancer Model; Demonstrating that CD44 is Required for BCSC Self-Renewal and Maintenance and for BCSC Growth In Vivo

Studies have shown that mammospheres are enriched for tumorigenic BCSCs (Al-Hajj et al., 2003; Reya et al., 2001). Three different preparations of primary mammospheres (MSSM-BCSC-1, -2, and -3) derived from fresh malignant human breast cancer tissues were established. These MSSM-BCSCs express high levels of the cancer stem cells marker, CD44, and low levels of CD24 (FIG. 28). They also express stem cell markers, Sox-2, Oct3/4, Nanog, and/or SSEA-1 (FIG. 28B and not shown), and display self-renewal capacity in the mammosphere formation assays (FIG. 28C-a-c) and tumorigenicity when implanted in immunocompromised Rag-1 (FIG. 28 E). As shown in FIG. 28A, several CD44 isoforms as well as the standard form of CD44 (CD44s, the lower band) are expressed by MSSM-BCSCs. We established a protocol to transduce BCSCs using retro- and lenti-viruses to express or to knock down expression of the genes of interests. These MSSM-BCSCs were transduced with the retroviruses carrying luciferase. After selection with G418, the drug resistant pooled populations of MSSM-BCSC-Luc cells express high levels of luciferase, which allowed tracking of their growth in vivo (FIG. 28E). Furthermore, it was found that shRNAs targeting CD44 expression inhibited mammosphere formation, while non-targeting shRNAs had no effect on mammosphere formation (FIG. 28C-d-f, D). This results demonstrates that CD44 is important for BCSC self-renewal and maintenance and that its target and dysfunction may help eliminate breast cancer stem cells and recurrence of the malignant disease.

### Example 21

### Purified CD44-Fc Fusion Proteins Inhibit In Vivo Growth of BCSCs

To assess the effect of purified hsCD44-Fc fusion proteins on BCSC growth in vivo, 1×10⁶ MSSM-BCSC-1 cells were injected subcutaneously into each Rag-1 mice. The tumors were allowed to growth for three weeks when the tumor volumes reach ˜200 mm³. The mice bearing similar size tumors were separated into 6 groups (6mice/group) and were treated with 4 intratumoral injections of 4 d/injection of 10 mg/ml of hsCD44s-Fc, hsCD44v8-v10-Fc, hsCD44v6-v10-Fc, hsCD44v3-v10-Fc, or human IgG, or 0.9% NaCl (FIG. 29). The experiments were stopped when the tumors of the control groups (treatment of human IgG or 0.9% NaCl) reached 1 cm in their longest diameters. All the tumors were dissected out and weighted. The results showed that CD44-Fc fusion proteins but not 0.9% NaCl or human IgG significantly inhibited growth of BCSCs in vivo (FIG. 29).

### Example 22

### CD44 is Up Regulated in the Stroma of Human Ovarian Cancer

To determine the contribution of CD44 to the progression of human ovarian cancer, available datasets at www.oncomine.org were mined and it was found that the CD44 transcript is up-regulated in human ovarian cancer comparing to normal ovary (FIG. 32A). Immunohistochemistry analyses indicated that CD44 and its ligand, HA, are up-regulated in the infiltrating stroma of stage III and IV of human ovarian cancers when compared to normal ovary (FIG. 32B-D and data not shown).

### Example 23

### CD44 is Important for Ovarian Cancer Stem Cell (OCSC) Self-Renewal and Maintenance

A series of in vivo selections by intraperitoneal (ip) implantation of parental SKOV3 and OVCAR-3 cells into Rag-1 immunocompromised mice to establish ascites ovarian cancer models were performed. SKOV3ip and OVCAR-3ip cells derived from these selections form subcutaneous as well as ascites tumors in Rag-1 mice (FIG. 34A and data not shown). In addition, CD44+ OCSC spheres (MSSM-OCSC-1 and -2) from fresh metastatic ovarian cancer tissues were generated. These MSSM-CSC cells express high levels of the cancer stem cells marker, CD44, and they also express the stem cell markers Sox-2, Oct3/4, and Nanog (FIG. 34B), and display self-renewal capacity in the sphere formation assays (FIG. 34E-a-c) and tumorigenicity when implanted in Rag-1 mice (FIG. 34C and not shown). We established a protocol to transduce OCSCs efficiently using retro- and lenti-viruses to express or to knock down expression of the genes of interests. It was found that shRNAs that knocked down CD44 expression (FIG. 34D), but not non-targeting shRNAs, inhibited sphere formation (FIG. 34E-d-f, 34F), demonstrating that CD44 is important for OCSC self-renewal and maintenance and its target may lead to eliminate ovarian cancer stem cells and stop disease recurrence.

### Example 24

### CD44 is Up-Regulated in Human Melanoma

To determine the contribution of CD44 to the progression of human melanoma, available datasets at www.oncomine.org were mined and it was found that the CD44 transcript is up-regulated in human melanoma comparing to normal skin (FIG. 35B). Western blot analysis also indicated that CD44 is up-regulated in human malignant melanoma cells when compared to normal melanocytes (FIG. 35C).

### Example 25

### hsCD44v3-v10-Fc, hsCD44v8-v10-Fc, and hsCD44s-Fc Inhibit Subcutaneous Growth of Human Melanoma Cells In Vivo

To assess the effects of expression of hsCD44-Fc fusion proteins on melanoma growth in vivo, 2×10⁶ M14 cells expressing different CD44-Fc fusion proteins (hsCD44s-Fc, hsCD44v8-v10-Fc, or hsCD44v3-v10-Fc) or transduced with empty expression vectors were injected subcutaneously into each Rag-1 mice. Tumors were allowed to grow for ˜4 weeks. At the end of experiments, all the tumors were dissected out and weighted. Data is presented as the mean of tumor weight (gram)+/−SD. The results showed that CD44-Fc fusion proteins especially hsCD44v3-v10-Fc significantly inhibited growth of M14 melanoma cells in vivo (FIG. 36B).

### Example 26

### CD44 is Up Regulated in Human Head-Neck Cancer

To determine the contribution of CD44 to the progression of human head-neck cancer, available datasets at www.oncomine.org were mined and it was found that the CD44 transcript is up-regulated in human head-neck cancer comparing to their normal counterparts (FIG. 38A, FIG. 39). Furthermore, CD44 expression in human head and neck carcinoma cells was assessed by Western blotting using anti-CD44 antibody (Santa Cruz). Expression level of CD44 by these carcinoma cells correlates with their tumorigenicity in vivo (FIG. 40A).

### Example 27

### hsCD44v3-v10-Fc, hsCD44v8-v10-Fc, and hsCD44s-Fc Inhibits Subcutaneous Growth of Human Head-Neck Cancer Cells In Vivo

To assess the effects of expression of hsCD44-Fc fusion proteins on head-neck cancer cell growth in vivo, 5×10⁶ SCC-4 cells expressing different CD44-Fc fusion proteins (hsCD44s-Fc, hsCD44v8-v10-Fc, or hsCD44v3-v10-Fc) or transduced with empty expression vectors were injected subcutaneously into each Rag-1 mice. Tumors were allowed to grow for ˜2 months. At the end of experiments, all the tumors were dissected out and weighted. Data is presented as the mean of tumor weight (gram)+/−SD. The results showed that CD44-Fc fusion proteins especially hsCD44v3-v10-Fc and hsCD44v8-v10-Fc significantly inhibited growth of SCC-4 cells in vivo (FIG. 40C).

### Example 28

### hsCD44v3-v10-Fc, hsCD44v8-v10-Fc, and hsCD44s-Fc Inhibits Subcutaneous Growth of Human Pancreatic and Liver Cancer Cells In Vivo

CD44 expression in human pancreatic and liver carcinoma cells was assessed by Western blotting using anti-CD44 antibody (Santa Cruz). The results showed that BXPC-3, PAN-08-13, PAN-08-27, PAN-10-05 pancreatic cancer cells and SK-HEP-1 liver cancer cells expression several CD44 isoforms (FIG. 41A).

To assess the effects of expression of hsCD44-Fc fusion proteins on in vivo growth of pancreatic and liver cancer cells, 5×10⁶ BXPC-3 and SK-HEP-1 cells expressing different CD44-Fc fusion proteins (hsCD44s-Fc, hsCD44v8-v10-Fc, or hsCD44v3-v10-Fc) or transduced with empty expression vectors were injected subcutaneously into each Rag-1 mice. Tumors were allowed to grow for ˜5 weeks. At the end of experiments, all the tumors were dissected out and weighted. Data is presented as the mean of tumor weight (gram)+/−SD. The results showed that CD44-Fc fusion proteins significantly inhibited in vivo growth of BXPC-3 and SK-HEP-1 (FIG. 41C-D).

### Example 29

### Methods of Detecting HA Using Biotin-Labeled CD44-Fc Fusion Proteins and Methods of Diagnosing Cancers by Detecting HA

Purified CD44-Fc fusion proteins (hsCD44s-Fc, hsCD44v8-v10-Fc, and hsCD44v3-v10-Fc) were labeled with biotin using EZ-Link Biotinylation Kits (Thermo Scientific) following the manufacturer's instruction. Human tumor paraffin sections were deparaffinized and rehydrated. After blocking with 2% BSA, the sections were incubated with biotinylated CD44-Fc fusion proteins (bCD44-Fc, 1 μg/ml) for overnight at 4 degree. Biotinylated CD44-Fc fusion proteins were detected by VECTASTAIN ABC kit. Our results showed that HA is up-regulated in stroma of malignant breast cancer and metastatic ovarian cancer (FIG. 25E, FIG. 32D) when compared to stroma of normal breast or ovarian (FIG. 25 D and data not shown). The bCD44-Fc positive staining is specific for HA as pre-treatment of the tissue sections with hyaluronidase eliminates the staining (data not shown).

It has been well established that HA is up-regulated in many cancer types including breast, ovarian, gladder cancer, and prostate cancers [for review see (Simpson and Lokeshwar, 2008; Tammi et al., 2008; Toole, 2004)](Golshani et al., 2008). HA level is correlated to tumor progression and metastasis (Toole and Hascall, 2002). Increased HA correlates with poor prognosis, disease progression, and shortened overall and disease specific survival in gastrointestinal tract, breast and ovary carcinoma (Anttila et al., 2000; Tammi et al., 2008). A study has shown that urinary HA measurement is an accurate marker for diagnosing bladder cancer (Lokeshwar et al., 2000).

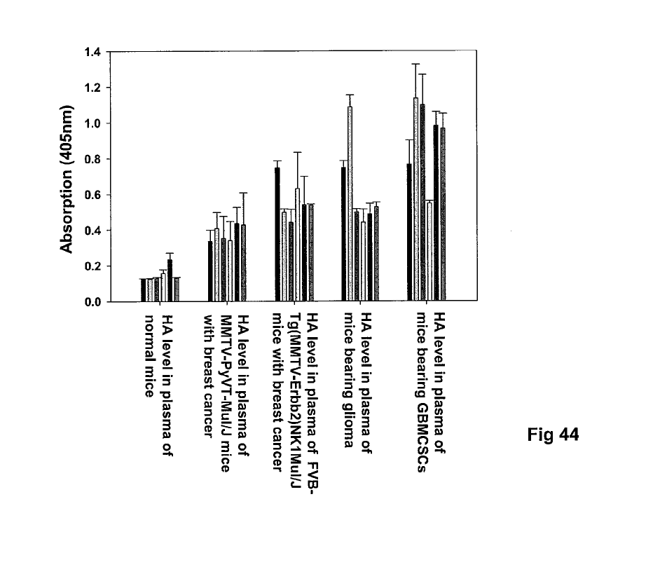

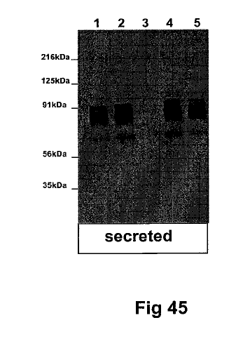

To detect plasma HA level, 200 μl blood from each transgenic mice (MMTV-PyVT and MMTV-ActErbb2, Jackson Lab) bearing breast cancer, each Rag-1 mice bearing gliomas derived from MSSM-GBMCSC-1 or Glioma 261 cells, or each control health mice were collected. Blood samples from six mouse of each type were collected and plasmas were generated immediately. 50 μl plasma from each sample was loaded in triplicate into each well of an Elisa plate that has been pre-coated with CD44-Fc fusion proteins. The CD44-Fc bound HA was detected by biotinylated CD44-Fc fusion proteins and AP-conjugated avidin. The developed color was measure by an Elisa machine at 405 nm. The results showed that HA is up-regulated in the plasma samples derived from mice bearing tumors when compared to the health mice (FIG. 44), demonstrating biotinylated CD44-Fc fusion proteins can be used to detect HA levels in plasma, serum, and urine of cancer patients and serve as diagnostic and prognostic reagent.

### Example 30

### Reduced CD44 Expression Sensitizes Prostate Cells to Cytotoxic Drugs In Vivo

The first-line and second-line cytotoxic drugs for prostate cancer are docetaxel, mitoxantrone, satraplatin, and ixabepilone. Mice will be injected subcutaneously with PC3/M-Luc and 22Rv1-Luc cells, depleted or not of endogenous CD44, and will be treated sequentially with docetaxel or mitoxantrone.

### Example 31

### Antagonists of CD44-hsCD44v3-v10-Fc, hsCD44v6-v10-Fc, hsCD44v8-v10-Fc, and hsCD44s-Fc-Serve as Effective Therapeutic Agents Against Various Cancers in Mouse Models

To determine whether antagonists of CD44 can be used to inhibit progression of a variety of human cancers in preclinical mouse models, soluble CD44 fusion proteins, such as CD44v3-v10-Fc, CD44v6-v10-Fc, CD44v8-v10-Fc, CD44v6-v10-Fc, or CD44s will be tested in different cancer mouse models.

These CD44-Fc fusion cDNAs have been inserted into retroviral vectors (Clontech) that contain the IRES element positioned between the cDNA inserts and the puromycin-resistance gene, so that all the puromycin-resistant cells are expected to express the inserted fusion genes. Human cancer cells, MEWO and A375 human melanoma cells; Lovo human colon cancer cells; Panc-1, HPAC, MIA PaCa-2, and/or AsPC-1 human pancreatic cancer cells; Hep 3B2.1-7 human hepatoma cells; SCC, -9, -15, and/or -25, human head and neck squamous carcinoma cells; U266 and MC/CAR human multiple myeloma cells; SKOV3ip and OVCAR -3ip human ovarian cancer cells; and 22Rv1 human prostate cancer cells; A549, LX529, NCI-H460, and/or NCI-H125 human lung cancer cells; MX-2 and/or SW613 human breast cancer cells; SKN-MC and A673 human sarcoma; H-MESO-1, H-MESO-1A, or MSTO-211H human malignant mesothelioma cells; and/or human cancer stem cells of different origins, will be transduced with retroviruses carrying the expression constructs encoding these fusion proteins or empty expression vector. After selection of the infected cells with puromycin, the pooled drug-resistant cancer cells will express high levels of CD44 fusion proteins, such as hsCD44v3-v10-Fc, hsCD44v6-v10-Fc, hsCD44v8-v10-Fc, and hsCD44s-Fc. These cells will be used to assess their ability to grow in Rag-1 mice and their response to chemotherapy and other targeted therapies.

Additional in vitro tumor cell viability experiments will be performed using CD44 depletion and/or hsCD44-Fc fusion proteins alone or in combination with the chemotherapeutic agent/RTK inhibitors/IAP inhibitors/p53 activator to determine whether CD44 antagonists sensitize the response of a variety of tumor cells to chemotherapy and other targeted therapies.

### DISCUSSION

In summary, the present Examples and figures demonstrate that CD44 is up-regulated in several human cancer types including human glioblastoma, colon cancer, ovarian cancer, head and neck squamous carcinoma, renal cell carcinoma, breast cancer, prostate cancer, gastric cancer, melanoma, and esophageal cancer. CD44 antagonists including shRNAs against human CD44 and/or a variety of CD44-Fc fusion proteins inhibit in vivo growth of human glioblastoma, colon, breast, prostate, lung, melanoma, pancreatic cancer, liver cancer, head and neck carcinoma, pancreatic, and ovarian cancers in mouse models. Moreover, the Examples demonstrate that CD44 is upregulated in human GBM and that knockdown of CD44 inhibits GBM growth in vivo by inhibiting glioma cell proliferation and promoting apoptosis. In addition, the Examples show for the first time that depletion of CD44 or CD44-Fc fusion proteins sensitizes GBM cells to chemotherapeutic and targeted agents in vivo, rendering it an attractive therapeutic target for gliomas, colon, breast, prostate, lung, melanoma, pancreatic cancer, liver cancer, head and neck carcinoma, and ovarian cancers. CD44 antagonists, in the form of human soluble CD44-Fc fusion proteins, such as hsCD44s-Fc, hsCD44v6-v10-Fc, hsCD44v8-v10-Fc, or hsCD44v3-v10-FC, and CD44-specific shRNAs proved to be effective therapeutic agents in inhibiting growth of human glioblastoma, colon, breast, prostate, lung, melanoma, pancreatic cancer, liver cancer, head and neck carcinoma, and ovarian cancers in mouse models. shRNAs of CD44 can also be used as gene therapy and delivered by nanoparticles.

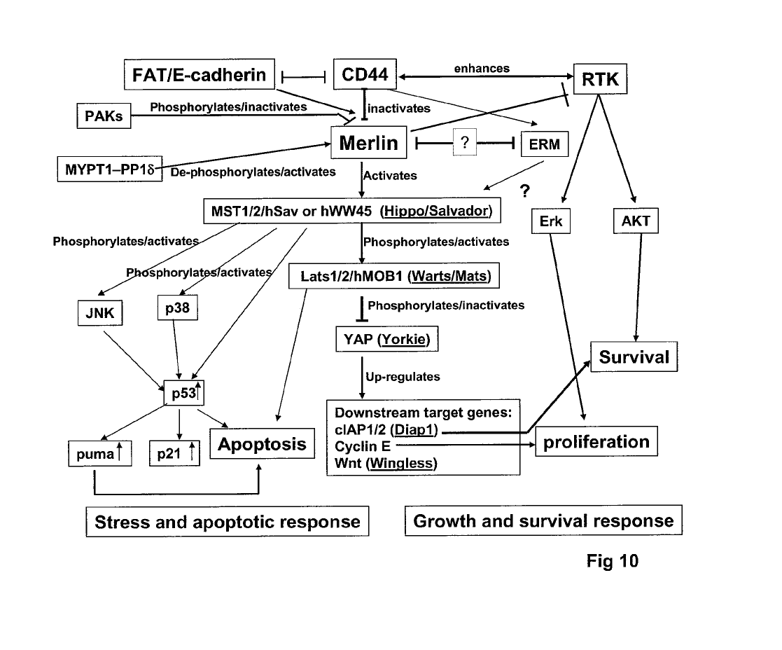

The present Examples demonstrate for the first time that CD44 functions upstream of mammalian Hippo stress and apoptotic signaling pathway (merlin-MST1/2-Lats1/2-YAP-cIAP1/2) and of two other downstream stress kinases, JNK and/or p38, along with their effectors, p53, and caspases (FIG. 5 and FIG. 6). They also provide evidence that CD44 plays an essential role in attenuating activation of stress and apoptotic signaling pathways induced by chemotherapeutic agents and reactive oxygen species (ROS) whereas loss of CD44 function leads to their sustained activation that promotes apoptosis of GBM cells and other cancer cells (see working model in FIG. 10).

These Examples show that depletion of CD44 inhibits Erk1/2 activation induced by EGFR ligands and HGF but not by NGF or FBS (FIG. 7), suggesting that CD44 serves as a co-receptor for these RTKs and enhances their signaling activity in malignant glioma cells and other cancer cells alike. Although the precise mechanism whereby CD44 regulates RTK signaling requires further investigation, its function as an HA receptor provides a possible explanation. CD44 forms large aggregates on the cell surface upon engagement by its multivalent ligand, HA. These aggregates often reside in lipid rafts or other specialized membrane domains where initiation of multiple signaling events occurs. In addition, CD44 can be expressed as a cell surface proteoglycan that binds numerous heparin binding growth factors including HB-EGF and basic FGF. As an RTK co-receptor, CD44 can therefore enhance signaling by facilitating RTK oligomerization and presenting the appropriate ligands to the corresponding RTKs. The ability of CD44v3-v1-Fc fusion proteins to modulate bioactivity of heparin binding growth factors, which include EGF family ligands, tumor angiogenic factors such as VEGF, bFGF, and angiopoietins, suggests that CD44 antagonists can be used to successfully in many combination therapies that target these ligands, their corresponding RTKs, and their downstream signaling pathways.

CD44 antagonists, hsCD44-Fc fusion proteins, and in particular hsCD44s-Fc, hsCD44v8-v10-Fc, or hsCD44v3-v10-Fc constructs, displayed potent activity against GBM, breast cancer, prostate cancer, melanoma, head-neck cancer, liver cancer, and pancreatic cancer in mouse models and inhibited self-renewal of breast and ovarian cancer stem cells, which offers hope for eradicating these deadly cancers in the future. Human sCD44-Fc fusion proteins may not only interfere with the function of CD44 expressed by GBM cells and other cancer cells but also with that expressed by host cells infiltrated the tumors. These host cells likely provide an essential contribution to progression of these cancer types similar to types of the effects of other molecules on cancers (Budhu et al., 2006; Orimo et al., 2005).

Currently available first line treatment options for human GBM are chemo- and radiation therapy, although both are largely palliative (Chamberlain, 2006). Effective treatments for malignant melanoma, lung cancer, and pancreatic cancer are almost not existed. There is also lack of effective treatment for liver cancer, head-neck cancer, late stage colon cancer, late stage/drug-resistant breast and prostate cancer. One hope for a better clinical outcome is to identify targets that play essential roles in mediating the microenvironment-derived survival signal and drug-resistance and that their antagonists can sensitize responses of these tumor cells to radiation, chemotherapeutic and targeted drugs. The Examples show that CD44 plays an important role in protecting cancer cells from oxidative and cytotoxic stress-induced apoptotic signaling while enhancing RTK signaling suggesting that CD44 may serve as an ideal therapeutic target to sensitize malignant glioma and other types of cancer cells to radiation, chemotherapy, and targeted therapies.

These Examples and Figures indicated that CD44 is a prime target for a variety of human cancer types including but not limited to human glioblastoma, colon cancer, breast cancer, prostate cancer, lung cancer, melanoma, head-neck cancer, liver cancer, pancreatic cancer, and ovarian cancer that CD44 antagonists including CD44-Fc fusion proteins and shRNAs are potent anti-cancer agents when used as single agents and in combinations with chemo- and/or radiation therapy, and the targeted therapies against erbB receptors, c-Met, IAPs, and activating p53. These Examples and Figures also demonstrate that CD44-Fc fusion proteins sensitize cancer cells to such cytotoxic agents such as chemo- and/or radiation therapies. Therefore, these fusion proteins are particularly amenable to being combined with such agents that will induce and/or promote stresses in tumor cells.

These Examples and Figures also show that these CD44-Fc fusion proteins bind specifically to HA and therefore can be used to detect HA in tissues section and in body fluids (blood, plasma, serum, and urine) for example in cancerous tissue. As a result, these fusion proteins can be used to diagnose cancers in which HA levels are elevated, which may lead to earlier detection of cancer then currently available methods and save lives. These fusion proteins can also be used to detect elevated HA levels, which will valuable in prognosis and early assessments of efficacy of therapeutic treatments, likely leading to more effective personalized treatment plans that increase overall survival of patients. The level of CD44 in these tumor samples and body fluid samples can be assessed in conjunction with HA levels to achieve more accurate predictions. Measuring HA and CD44 levels can be done using standard immunological techniques and detection methods.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

### REFERENCES

Abounader, R., and Laterra, J. (2005). Scatter factor/hepatocyte growth factor in brain tumor growth and angiogenesis. Neuro Oncol 7, 436-451.
Ahrens, T., Sleeman, J. P., Schempp, C. M., Howells, N., Hofmann, M., Ponta, H., Herrlich, P., and Simon, J. C. (2001). Soluble CD44 inhibits melanoma tumor growth by blocking cell surface CD44 binding to hyaluronic acid. Oncogene 20, 3399-3408.
Ailles, L., and Prince, M. (2009). Cancer stem cells in head and neck squamous cell carcinoma. Methods Mol Biol 568, 175-193.
Al-Hajj, M., Wicha, M. S., Benito-Hernandez, A., Morrison, S. J., and Clarke, M. F. (2003). Prospective identification of tumorigenic breast cancer cells. Proc Natl Acad Sci USA 100, 3983-3988.
Alves, C. S., Burdick, M. M., Thomas, S. N., Pawar, P., and Konstantopoulos, K. (2008). The dual role of CD44 as a functional P-selectin ligand and fibrin receptor in colon carcinoma cell adhesion. Am J Physiol Cell Physiol 294, C907-916.
Anttila, M. A., Tammi, R. H., Tammi, M. I., Syrjanen, K. J., Saarikoski, S. V., and Kosma, V. M. (2000). High levels of stromal hyaluronan predict poor disease outcome in epithelial ovarian cancer. Cancer Res 60, 150-155.
Araujo, R. P., Liotta, L. A., and Petricoin, E. F. (2007). Proteins, drug targets and the mechanisms they control: the simple truth about complex networks. Nat Rev Drug Discov 6, 871-880.
Bai, Y., Liu, Y. J., Wang, H., Xu, Y., Stamenkovic, I., and Yu, Q. (2007). Inhibition of the hyaluronan-CD44 interaction by merlin contributes to the tumor-suppressor activity of merlin. Oncogene 26, 836-850.
Banerji, S., Wright, A. J., Noble, M., Mahoney, D. J., Campbell, I. D., Day, A. J., and Jackson, D. G. (2007). Structures of the Cd44-hyaluronan complex provide insight into a fundamental carbohydrate-protein interaction. Nat Struct Mol Biol 14, 234-239.
Barbour, A. P., Reeder, J. A., Walsh, M. D., Fawcett, J., Antalis, T. M., and Gotley, D. C. (2003). Expression of the CD44v2-10 isoform confers a metastatic phenotype: importance of the heparan sulfate attachment site CD44v3. Cancer Res 63, 887-892.
Bennett, K. L., Jackson, D. G., Simon, J. C., Tanczos, E., Peach, R., Modrell, B., Stamenkovic, I., Plowman, G., and Aruffo, A. (1995). CD44 isoforms containing exon V3 are responsible for the presentation of heparin-binding growth factor. J Cell Biol 128, 687-698.
Bredel, M., Bredel, C., Juric, D., Harsh, G. R., Vogel, H., Recht, L. D., and Sikic, B. I. (2005). Functional network analysis reveals extended gliomagenesis pathway maps and three novel MYC-interacting genes in human gliomas. Cancer Res 65, 8679-8689.
Budhu, A., Forgues, M., Ye, Q. H., Jia, H. L., He, P., Zanetti, K. A., Kammula, U.S., Chen, Y., Qin, L. X., Tang, Z. Y., et al. (2006). Prediction of venous metastases, recurrence, and prognosis in hepatocellular carcinoma based on a unique immune response signature of the liver microenvironment. Cancer Cell 10, 99-111.
Campos, S., Hamid, O., Seiden, M. V., Oza, A., Plante, M., Potkul, R. K., Lenehan, P. F., Kaldjian, E. P., Varterasian, M. L., Jordan, C., et al. (2005). Multicenter, randomized phase II trial of oral CI-1033 for previously treated advanced ovarian cancer. J Clin Oncol 23, 5597-5604.
Chamberlain, M. C. (2006). Treatment options for glioblastoma. Neurosurg Focus 20, E19.
Collins, A. T., Berry, P. A., Hyde, C., Stower, M. J., and Maitland, N. J. (2005). Prospective identification of tumorigenic prostate cancer stem cells. Cancer Res 65, 10946-10951.
Croker, A K., and Allan, A. L. (2008). Cancer stem cells: implications for the progression and treatment of metastatic disease. J Cell Mol Med 12, 374-390.
Davis, F. G., Freels, S., Grutsch, J., Barlas, S., and Brem, S. (1998). Survival rates in patients with primary malignant brain tumors stratified by patient age and tumor histological type: an analysis based on Surveillance, Epidemiology, and End Results (SEER) data, 1973-1991. J Neurosurg 88, 1-10.
Dean, M., Fojo, T., and Bates, S. (2005). Tumour stem cells and drug resistance. Nat Rev Cancer 5, 275-284.
Dimitroff, C. J., Lee, J. Y., Rafii, S., Fuhlbrigge, R. C., and Sackstein, R. (2001). CD44 is a major E-selectin ligand on human hematopoietic progenitor cells. J Cell Biol 153, 1277-1286.
Du, L., Wang, H., He, L., Zhang, J., Ni, B., Wang, X., Jin, H., Cahuzac, N., Mehrpour, M., Lu, Y., et al. (2008). CD44 is of functional importance for colorectal cancer stem cells. Clin Cancer Res 14, 6751-6760.
Eskens, F. A., Mom, C. H., Planting, A. S., Gietema, J. A., Amelsberg, A., Huisman, H., van Doom, L., Burger, H., Stopfer, P., Verweij, J., et al. (2008). A phase I dose escalation study of BIBW 2992, an irreversible dual inhibitor of epidermal growth factor receptor 1 (EGFR) and 2 (HER2) tyrosine kinase in a 2-week on, 2-week off schedule in patients with advanced solid tumours. Br J Cancer 98, 80-85.
Ghanem, M. A., Van Steenbrugge, G. J., Van Der Kwast, T. H., Sudaryo, M. K., Noordzij, M. A., and Nijman, R. J. (2002). Expression and prognostic value Of CD44 isoforms in nephroblastoma (Wilms tumor). J Urol 168, 681-686.
Giese, A., Bjerkvig, R., Berens, M. E., and Westphal, M. (2003). Cost of migration: invasion of malignant gliomas and implications for treatment. J Clin Oncol 21, 1624-1636.
Gilbertson, R. J., and Rich, J. N. (2007). Making a tumour's bed: glioblastoma stem cells and the vascular niche. Nat Rev Cancer 7, 733-736.
Ginos, M. A., Page, G. P., Michalowicz, B. S., Patel, K. J., Volker, S. E., Pambuccian, S. E., Ondrey, F. G., Adams, G. L., and Gaffney, P. M. (2004). Identification of a gene expression signature associated with recurrent disease in squamous cell carcinoma of the head and neck. Cancer Res 64, 55-63.
Golshani, R., Lopez, L., Estrella, V., Kramer, M., Iida, N., and Lokeshwar, V. B. (2008). Hyaluronic acid synthase-1 expression regulates bladder cancer growth, invasion, and angiogenesis through CD44. Cancer Res 68, 483-491.
Graudens, E., Boulanger, V., Mollard, C., Mariage-Samson, R., Barlet, X., Gremy, G., Couillault, C., Lajemi, M., Piatier-Tonneau, D., Zaborski, P., et al. (2006). Deciphering cellular states of innate tumor drug responses. Genome Biol 7, R19.
Gumz, M. L., Zou, H., Kreinest, P. A., Childs, A. C., Belmonte, L. S., LeGrand, S. N., Wu, K. J., Luxon, B. A., Sinha, M., Parker, A. S., et al. (2007). Secreted frizzled-related protein 1 loss contributes to tumor phenotype of clear cell renal cell carcinoma. Clin Cancer Res 13, 4740-4749.
Guo, Y., Ma, J., Wang, J., Che, X., Narula, J., Bigby, M., Wu, M., and Sy, M. S. (1994). Inhibition of human melanoma growth and metastasis in vivo by anti-CD44 monoclonal antibody. Cancer Res 54, 1561-1565.
Gutmann, D. H., Giordano, M. J., Fishback, A. S., and Guha, A. (1997). Loss of merlin expression in sporadic meningiomas, ependymomas and schwannomas. Neurology 49, 267-270.
Hamaratoglu, F., Willecke, M., Kango-Singh, M., Nolo, R., Hyun, E., Tao, C., Jafar-Nejad, H., and Halder, G. (2006). The tumour-suppressor genes NF2/Merlin and Expanded act through Hippo signalling to regulate cell proliferation and apoptosis. Nat Cell Biol 8, 27-36.
Hambardzumyan, D., Squatrito, M., Carbajal, E., and Holland, E. C. (2008). Glioma formation, cancer stem cells, and akt signaling. Stem Cell Rev 4, 203-210.
Hao, Y., Chun, A., Cheung, K., Rashidi, B., and Yang, X. (2008). Tumor suppressor LATS1 is a negative regulator of oncogene YAP. J Biol Chem 283, 5496-5509.
Hauptschein, R. S., Sloan, K. E., Torella, C., Moezzifard, R., Giel-Moloney, M., Zehetmeier, C., Unger, C., Ilag, L. L., and Jay, D. G. (2005). Functional proteomic screen identifies a modulating role for CD44 in death receptor-mediated apoptosis. Cancer Res 65, 1887-1896.
Hendrix, N. D., Wu, R., Kuick, R., Schwartz, D. R., Fearon, E. R., and Cho, K. R. (2006). Fibroblast growth factor 9 has oncogenic activity and is a downstream target of Wnt signaling in ovarian endometrioid adenocarcinomas. Cancer Res 66, 1354-1362.
Hidalgo, A., Peired, A. J., Wild, M. K., Vestweber, D., and Eremite, P. S. (2007). Complete identification of E-selectin ligands on neutrophils reveals distinct functions of PSGL-1, ESL-1, and CD44. Immunity 26, 477-489.
Hideshima, T., Mitsiades, C., Tonon, G., Richardson, P. G., and Anderson, K. C. (2007). Understanding multiple myeloma pathogenesis in the bone marrow to identify new therapeutic targets. Nat Rev Cancer 7, 585-598.
Hoelzinger, D. B., Demuth, T., and Berens, M. E. (2007). Autocrine factors that sustain glioma invasion and paracrine biology in the brain microenvironment. J Natl Cancer Inst 99, 1583-1593.
Holash, J., Davis, S., Papadopoulos, N., Croll, S. D., Ho, L., Russell, M., Boland, P., Leidich, R., Hylton, D., Burova, E., et al. (2002). VEGF-Trap: a VEGF blocker with potent antitumor effects. Proc Natl Acad Sci USA 99, 11393-11398.
Hong, S. P., Wen, J., Bang, S., Park, S., and Song, S. Y. (2009). CD44-positive cells are responsible for gemcitabine resistance in pancreatic cancer cells. Int J Cancer.
Huang, J., Wu, S., Barrera, J., Matthews, K., and Pan, D. (2005). The Hippo signaling pathway coordinately regulates cell proliferation and apoptosis by inactivating Yorkie, the Drosophila Homolog of YAP. Cell 122, 421-434.
Hurt, E. M., Kawasaki, B. T., Klarmann, G. J., Thomas, S. B., and Farrar, W. L. (2008). CD44+ CD24(−) prostate cells are early cancer progenitor/stem cells that provide a model for patients with poor prognosis. Br J Cancer 98, 756-765.
Jin, L., Hope, K. J., Zhai, Q., Smadja-Joffe, F., and Dick, J. E. (2006). Targeting of CD44 eradicates human acute myeloid leukemic stem cells. Nat Med 12, 1167-1174.
Kajita, M., Itoh, Y., Chiba, T., Mori, H., Okada, A., Kinoh, H., and Seiki, M. (2001). Membrane-type 1 matrix metalloproteinase cleaves CD44 and promotes cell migration. J Cell Biol 153, 893-904.
Katayama, Y., Hidalgo, A., Chang, J., Peired, A., and Frenette, P. S. (2005). CD44 is a physiological E-selectin ligand on neutrophils. J Exp Med 201, 1183-1189.
Kim, E. S., Serur, A., Huang, J., Manley, C. A., McCrudden, K. W., Frischer, J. S., Soffer, S. Z., Ring, L., New, T., Zabski, S., et al. (2002). Potent VEGF blockade causes regression of coopted vessels in a model of neuroblastoma. Proc Natl Acad Sci USA 99, 11399-11404.
Klingbeil, P., Marhaba, R., Jung, T., Kirmse, R., Ludwig, T., and Zoller, M. (2009). CD44 variant isoforms promote metastasis formation by a tumor cell-matrix cross-talk that supports adhesion and apoptosis resistance. Mol Cancer Res 7, 168-179.
Kluwe, L., Bayer, S., Baser, M. E., Hazim, W., Haase, W., Funsterer, C., and Mautner, V. F. (1996). Identification of NF2 germ-line mutations and comparison with neurofibromatosis 2 phenotypes. Hum Genet 98, 534-538.
Krause, D. S., Lazarides, K., von Andrian, U. H., and Van Etten, R. A. (2006). Requirement for CD44 in homing and engraftment of BCR-ABL-expressing leukemic stem cells. Nat Med 12, 1175-1180.
Lau, Y. K., Murray, L. B., Houshmandi, S. S., Xu, Y., Gutmann, D. H., and Yu, Q. (2008). Merlin is a potent inhibitor of glioma growth. Cancer Res 68, 5733-5742.
Lee, C. J., Dosch, J., and Simeone, D. M. (2008). Pancreatic cancer stem cells. J Clin Oncol 26, 2806-2812.
Lehtinen, M. K., Yuan, Z., Boag, P. R., Yang, Y., Villen, J., Becker, E. B., DiBacco, S., de la Iglesia, N., Gygi, S., Blackwell, T. K., et al. (2006). A conserved MST-FOXO signaling pathway mediates oxidative-stress responses and extends life span. Cell 125, 987-1001.
Li, D. M., Li, S. S., Zhang, Y. H., Zhang, H. J., Gao, D. L., and Wang, Y. X. (2005). Expression of human chorionic gonadotropin, CD44v6 and CD44v4/5 in esophageal squamous cell carcinoma. World J Gastroenterol 11, 7401-7404.
Liang, Y., Diehn, M., Watson, N., Bollen, A. W., Aldape, K. D., Nicholas, M. K., Lamborn, K. R., Berger, M. S., Botstein, D., Brown, P. O., et al. (2005). Gene expression profiling reveals molecularly and clinically distinct subtypes of glioblastoma multiforme. Proc Natl Acad Sci USA 102, 5814-5819.
Lim, S. D., Young, A. N., Paner, G. P., and Amin, M. B. (2008). Prognostic role of CD44 cell adhesion molecule expression in primary and metastatic renal cell carcinoma: a clinicopathologic study of 125 cases. Virchows Arch 452, 49-55.
Ling, P., Lu, T. J., Yuan, C. J., and Lai, M. D. (2008). Biosignaling of mammalian Ste20-related kinases. Cell Signal 20, 1237-1247.
Liu, R., Wang, X., Chen, G. Y., Dalerba, P., Gurney, A., Hoey, T., Sherlock, G., Lewicki, J., Shedden, K., and Clarke, M. F. (2007). The prognostic role of a gene signature from tumorigenic breast-cancer cells. N Engl J Med 356, 217-226.
Lokeshwar, V. B., Obek, C., Pham, H. T., Wei, D., Young, M. J., Duncan, R. C., Soloway, M. S., and Block, N. L. (2000). Urinary hyaluronic acid and hyaluronidase: markers for bladder cancer detection and evaluation of grade. J Urol 163, 348-356.
Lucin, K., Matusan, K., Dordevic, G., and Stipic, D. (2004). Prognostic significance of CD44 molecule in renal cell carcinoma. Croat Med J 45, 703-708.
Maher, E. A., Furnari, F. B., Bachoo, R. M., Rowitch, D. H., Louis, D. N., Cavenee, W. K., and DePinho, R. A. (2001). Malignant glioma: genetics and biology of a grave matter. Genes Dev 15, 1311-1333.
Maitland, N. J., and Collins, A. T. (2008). Prostate cancer stem cells: a new target for therapy. J Clin Oncol 26, 2862-2870.
Mantovani, A., Allavena, P., Sica, A., and Balkwill, F. (2008). Cancer-related inflammation. Nature 454, 436-444.
Marastoni, S., Ligresti, G., Lorenzon, E., Colombatti, A., and Mongiat, M. (2008). Extracellular matrix: a matter of life and death. Connect Tissue Res 49, 203-206.
Matallanas, D., Romano, D., Hamilton, G., Kolch, W., and O'Neill, E. (2008). A Hippo in the ointment: MST signalling beyond the fly. Cell Cycle 7, 879-884.
Matsumura, Y., and Tarin, D. (1992). Significance of CD44 gene products for cancer diagnosis and disease evaluation. Lancet 340, 1053-1058.
Matzke, A., Sargsyan, V., Hohmann, B., Aramuni, G., Asan, E., Sendtner, M., Pace, G., Howells, N., Zhang, W., Ponta, H., et al. (2007). Haploinsufficiency of c-Met in cd44−/−mice identifies a collaboration of CD44 and c-Met in vivo. Mol Cell Biol 27, 8797-8806.
McClatchey, A. I., and Giovannini, M. (2005). Membrane organization and tumorigenesis—the NF2 tumor suppressor, Merlin. Genes Dev 19, 2265-2277.
Mishra, P. J., Mishra, P. J., Glod, J. W., and Banerjee, D. (2009). Mesenchymal stem cells: flip side of the coin. Cancer Res 69, 1255-1258.
Mitsiades, C. S. (2005). CD44v6, a target for novel antibody treatment approaches, is frequently expressed in multiple myeloma and associated with deletion of chromosome arm 13q. Haematologica 90, 436-437.
Morrison, H., Sherman, L. S., Legg, J., Banine, F., Isacke, C., Haipek, C. A., Gutmann, D. H., Ponta, H., and Herrlich, P. (2001). The NF2 tumor suppressor gene product, merlin, mediates contact inhibition of growth through interactions with CD44. Genes Dev 15, 968-980.
Murai, T., Miyazaki, Y., Nishinakamura, H., Sugahara, K. N., Miyauchi, T., Sako, Y., Yanagida, T., and Miyasaka, M. (2004). Engagement of CD44 promotes Rac activation and CD44 cleavage during tumor cell migration. J Biol Chem 279, 4541-4550.
Naor, D., Sionov, R. V., and Ish-Shalom, D. (1997). CD44: structure, function, and association with the malignant process. Adv Cancer Res 71, 241-319.
Nelson, A. D., and Grandis, J. R. (2007). The role of CD44 in HNSCC. Cancer Biol Ther 6, 125-126.
Nemunaitis, J., Eiseman, I., Cunningham, C., Senzer, N., Williams, A., Lenehan, P. F., Olson, S. C., Bycott, P., Schlicht, M., Zentgraff, R., et al. (2005). Phase 1 clinical and pharmacokinetics evaluation of oral CI-1033 in patients with refractory cancer. Clin Cancer Res 11, 3846-3853.
Notterman, D. A., Alon, U., Sierk, A. J., and Levine, A. J. (2001). Transcriptional gene expression profiles of colorectal adenoma, adenocarcinoma, and normal tissue examined by oligonucleotide arrays. Cancer Res 61, 3124-3130.
Nozoe, T., Kohnoe, S., Ezaki, T., Kabashima, A., and Maehara, Y. (2004). Significance of immunohistochemical over-expression of CD44v6 as an indicator of malignant potential in esophageal squamous cell carcinoma. J Cancer Res Clin Oncol 130, 334-338.
Ohwada, C., Nakaseko, C., Koizumi, M., Takeuchi, M., Ozawa, S., Naito, M., Tanaka, H., Oda, K., Cho, R., Nishimura, M., et al. (2008). CD44 and hyaluronan engagement promotes dexamethasone resistance in human myeloma cells. Eur J Haematol 80, 245-250.
Okada, T., You, L., and Giancotti, F. G. (2007). Shedding light on Merlin's wizardry. Trends Cell Biol 17, 222-229.
Oost, T. K., Sun, C., Armstrong, R. C., Al-Assaad, A. S., Betz, S. F., Deckwerth, T. L., Ding, H., Elmore, S. W., Meadows, R. P., Olejniczak, E. T., et al. (2004). Discovery of potent antagonists of the antiapoptotic protein XIAP for the treatment of cancer. J Med Chem 47, 4417-4426.
Orian-Rousseau, V., Chen, L., Sleeman, J. P., Herrlich, P., and Ponta, H. (2002). CD44 is required for two consecutive steps in HGF/c-Met signaling. Genes Dev 16, 3074-3086.
Orian-Rousseau, V., and Ponta, H. (2008). Adhesion proteins meet receptors: a common theme? Adv Cancer Res 101, 63-92.
Orimo, A., Gupta, P. B., Sgroi, D. C., Arenzana-Seisdedos, F., Delaunay, T., Naeem, R., Carey, V. J., Richardson, A. L., and Weinberg, R. A. (2005). Stromal fibroblasts present in invasive human breast carcinomas promote tumor growth and angiogenesis through elevated SDF-1/CXCL12 secretion. Cell 121, 335-348.
Overholtzer, M., Zhang, J., Smolen, G. A., Muir, B., Li, W., Sgroi, D. C., Deng, C. X., Brugge, J. S., and Haber, D. A. (2006). Transforming properties of YAP, a candidate oncogene on the chromosome 11q22 amplicon. Proc Natl Acad Sci USA 103, 12405-12410.
Pall, T., Gad, A., Kasak, L., Drews, M., Stromblad, S., and Kogerman, P. (2004). Recombinant CD44-HABD is a novel and potent direct angiogenesis inhibitor enforcing endothelial cell-specific growth inhibition independently of hyaluronic acid binding. Oncogene 23, 7874-7881.
Pals, S. T., Horst, E., Ossekoppele, G. J., Figdor, C. G., Scheper, R. J., and Meijer, C. J. (1989). Expression of lymphocyte homing receptor as a mechanism of dissemination in non-Hodgkin's lymphoma. Blood 73, 885-888.
Park, J. B., Kwak, H. J., and Lee, S. H. (2008). Role of hyaluronan in glioma invasion. Cell Adh Migr 2, 202-207.
Patrawala, L., Calhoun, T., Schneider-Broussard, R., Li, H., Bhatia, B., Tang, S., Reilly, J. G., Chandra, D., Zhou, J., Claypool, K., et al. (2006). Highly purified CD44+ prostate cancer cells from xenograft human tumors are enriched in tumorigenic and metastatic progenitor cells. Oncogene 25, 1696-1708.
Peach, R. J., Hollenbaugh, D., Stamenkovic, I., and Aruffo, A. (1993). Identification of hyaluronic acid binding sites in the extracellular domain of CD44. J Cell Biol 122, 257-264.
Pellock, B. J., Buff, E., White, K., and Hariharan, I. K. (2007). The Drosophila tumor suppressors Expanded and Merlin differentially regulate cell cycle exit, apoptosis, and Wingless signaling. Dev Biol 304, 102-115.
Pinkerton, H., and Rana, M. W. (1976). Cytotoxic effects of 1,3-bis(2 chloroethyl)-1-nitrosourea (BCNU) on cultured human glioblastomas. Proc Soc Exp Biol Med 151, 532-534.
Podar, K., Chauhan, D., and Anderson, K. C. (2009). Bone marrow microenvironment and the identification of new targets for myeloma therapy. Leukemia 23, 10-24.
Ponta, H., Sherman, L., and Herrlich, P. A. (2003). CD44: from adhesion molecules to signalling regulators. Nat Rev Mol Cell Biol 4, 33-45.
Ponta, H., Wainwright, D., and Herrlich, P. (1998). The CD44 protein family Int J Biochem Cell Biol 30, 299-305.
Ponti, D., Costa, A., Zaffaroni, N., Pratesi, G., Petrangolini, G., Coradini, D., Pilotti, S., Pierotti, M. A., and Daidone, M. G. (2005). Isolation and in vitro propagation of tumorigenic breast cancer cells with stem/progenitor cell properties. Cancer Res 65, 5506-5511.
Ramos-Nino, M. E., Blumen, S. R., Pass, H., and Mossman, B. T. (2007). Fra-1 governs cell migration via modulation of CD44 expression in human mesotheliomas. Mol Cancer 6, 81.
Reim, F., Dombrowski, Y., Ritter, C., Buttmann, M., Hausler, S., Ossadnik, M., Krockenberger, M., Beier, D., Beier, C. P., Dietl, J., et al. (2009). Immunoselection of Breast and Ovarian Cancer Cells with Trastuzumab and Natural Killer Cells: Selective Escape of CD44high/CD24low/HER2low Breast Cancer Stem Cells. Cancer Res 69, 8058-8066.
Reya, T., Duncan, A. W., Ailles, L., Domen, J., Scherer, D. C., Willert, K., Hintz, L., Nusse, R., and Weissman, I. L. (2003). A role for Wnt signalling in self-renewal of haematopoietic stem cells. Nature 423, 409-414.
Reya, T., Morrison, S. J., Clarke, M. F., and Weissman, I. L. (2001). Stem cells, cancer, and cancer stem cells. Nature 414, 105-111.
Sainio, M., Zhao, F., Heiska, L., Turunen, O., den Bakker, M., Zwarthoff, E., Lutchman, M., Rouleau, G. A., Jaaskelainen, J., Vaheri, A., et al. (1997). Neurofibromatosis 2 tumor suppressor protein colocalizes with ezrin and CD44 and associates with actin-containing cytoskeleton. J Cell Sci 110 (Pt 18), 2249-2260.
Screaton, G. R., Bell, M. V., Bell, J. I., and Jackson, D. G. (1993). The identification of a new alternative exon with highly restricted tissue expression in transcripts encoding the mouse Pgp-1 (CD44) homing receptor. Comparison of all 10 variable exons between mouse, human, and rat. J Biol Chem 268, 12235-12238.
Screaton, G. R., Bell, M. V., Jackson, D. G., Cornelis, F. B., Gerth, U., and Bell, J. I. (1992). Genomic structure of DNA encoding the lymphocyte homing receptor CD44 reveals at least 12 alternatively spliced exons. Proc Natl Acad Sci USA 89, 12160-12164.
Shai, R., Shi, T., Kremen, T. J., Horvath, S., Liau, L. M., Cloughesy, T. F., Mischel, P. S., and Nelson, S. F. (2003). Gene expression profiling identifies molecular subtypes of gliomas. Oncogene 22, 4918-4923.
Sharma, S. V., Bell, D. W., Settleman, J., and Haber, D. A. (2007). Epidermal growth factor receptor mutations in lung cancer. Nat Rev Cancer 7, 169-181.
Sherman, L., Sleeman, J., Herrlich, P., and Ponta, H. (1994). Hyaluronate receptors: key players in growth, differentiation, migration and tumor progression. Curr Opin Cell Biol 6, 726-733.
Shipitsin, M., Campbell, L. L., Argani, P., Weremowicz, S., Bloushtain-Qimron, N., Yao, J., Nikolskaya, T., Serebryiskaya, T., Beroukhim, R., Hu, M., et al. (2007). Molecular definition of breast tumor heterogeneity. Cancer Cell 11, 259-273.
Simpson, M. A., and Lokeshwar, V. B. (2008). Hyaluronan and hyaluronidase in genitourinary tumors. Front Biosci 13, 5664-5680.
Srinivasula, S. M., and Ashwell, J. D. (2008). IAPs: what's in a name? Mol Cell 30, 123-135.
Stamenkovic, I. (2000). Matrix metalloproteinases in tumor invasion and metastasis. Semin Cancer Biol 10, 415-433.
Stamenkovic, I., Amiot, M., Pesando, J. M., and Seed, B. (1989). A lymphocyte molecule implicated in lymph node homing is a member of the cartilage link protein family. Cell 56, 1057-1062.
Stamenkovic I, Y. Q. (2009). CD44 meets Merlin and Ezrin: Their Interplay Mediates the Pro-tumor Activity of CD44 and Tumor-Suppressing Effect of Merlin, Vol Chapter 5, pp 71-88 (Edited by Robert Stern). ELSEVIER PUBLISHING.
Stamenkovic, I., and Yu, Q. (2009). Shedding light on proteolytic cleavage of CD44: the responsible sheddase and functional significance of shedding. J Invest Dermatol 129, 1321-1324.
Stavropoulos, N. E., Filliadis, I., Ioachim, E., Michael, M., Mermiga, E., Hastazeris, K., and Nseyo, U. O. (2001). CD44 standard form expression as a predictor of progression in high risk superficial bladder tumors. Int Urol Nephrol 33, 479-483.
Sun, L., Hui, A. M., Su, Q., Vortmeyer, A., Kotliarov, Y., Pastorino, S., Passaniti, A., Menon, J., Walling, J., Bailey, R., et al. (2006). Neuronal and glioma-derived stem cell factor induces angiogenesis within the brain. Cancer Cell 9, 287-300.
Sy, M. S., Guo, Y. J., and Stamenkovic, I. (1992). Inhibition of tumor growth in vivo with a soluble CD44-immunoglobulin fusion protein. J Exp Med 176, 623-627.
Tajima, K., Ohashi, R., Sekido, Y., Hida, T., Nara, T., Hashimoto, M., Iwakami, S., Minakata, K., Yae, T., Takahashi, F., et al. (2010). Osteopontin-mediated enhanced hyaluronan binding induces multidrug resistance in mesothelioma cells. Oncogene 29, 1941-1951.
Takahashi, Y., Miyoshi, Y., Takahata, C., Irahara, N., Taguchi, T., Tamaki, Y., and Noguchi, S. (2005). Down-regulation of LATS1 and LATS2 mRNA expression by promoter hypermethylation and its association with biologically aggressive phenotype in human breast cancers. Clin Cancer Res 11, 1380-1385.
Tammi, R. H., Kultti, A., Kosma, V. M., Pirinen, R., Auvinen, P., and Tammi, M. I. (2008). Hyaluronan in human tumors: pathobiological and prognostic messages from cell-associated and stromal hyaluronan. Semin Cancer Biol 18, 288-295.
Toole, B. P. (2004). Hyaluronan: from extracellular glue to pericellular cue. Nat Rev Cancer 4, 528-539.
Toole, B. P., and Hascall, V. C. (2002). Hyaluronan and tumor growth. Am J Pathol 161, 745-747.
Tsukita, S., and Yonemura, S. (1997). ERM (ezrin/radixin/moesin) family: from cytoskeleton to signal transduction. Curr Opin Cell Biol 9, 70-75.
Turley, E. A., Noble, P. W., and Bourguignon, L. Y. (2002). Signaling properties of hyaluronan receptors. J Biol Chem 277, 4589-4592.
van der Voort, R., Taher, T. E., Wielenga, V. J., Spaargaren, M., Prevo, R., Smit, L., David, G., Hartmann, G., Gherardi, E., and Pals, S. T. (1999). Heparan sulfate-modified CD44 promotes hepatocyte growth factor/scatter factor-induced signal transduction through the receptor tyrosine kinase c-Met. J Biol Chem 274, 6499-6506.
Verhulst, A., Asselman, M., Persy, V. P., Schepers, M. S., Helbert, M. F., Verkoelen, C. F., and De Broe, M. E. (2003). Crystal retention capacity of cells in the human nephron: involvement of CD44 and its ligands hyaluronic acid and osteopontin in the transition of a crystal binding-into a nonadherent epithelium. J Am Soc Nephrol 14, 107-115.
Voelzke, W. R., Petty, W. J., and Lesser, G. J. (2008). Targeting the epidermal growth factor receptor in high-grade astrocytomas. Curr Treat Options Oncol 9, 23-31.
Xu, Y., Stamenkovic, I., and Yu, Q. (2010). CD44 attenuates activation of the hippo signaling pathway and is a prime therapeutic target for glioblastoma. Cancer Res 70, 2455-2464.
Xu, Y., and Yu, Q. (2003). E-cadherin negatively regulates CD44-hyaluronan interaction and CD44-mediated tumor invasion and branching morphogenesis. J Biol Chem 278, 8661-8668.
Yang, G. H., Fan, J., Xu, Y., Qiu, S. J., Yang, X. R., Shi, G. M., Wu, B., Dai, Z., Liu, Y. K., Tang, Z. Y., et al. (2008). Osteopontin combined with CD44, a novel prognostic biomarker for patients with hepatocellular carcinoma undergoing curative resection. Oncologist 13, 1155-1165.
Yildiz, E., Gokce, G., Kilicarslan, H., Ayan, S., Goze, O. F., and Gultekin, E. Y. (2004). Prognostic value of the expression of Ki-67, CD44 and vascular endothelial growth factor, and microvessel invasion, in renal cell carcinoma. BJU Int 93, 1087-1093.
Yoshida, M., Yasuda, T., Hiramitsu, T., Ito, H., and Nakamura, T. (2008). Induction of apoptosis by anti-CD44 antibody in human chondrosarcoma cell line SW1353. Biomed Res 29, 47-52.
Yu, Q., and Stamenkovic, I. (1999). Localization of matrix metalloproteinase 9 to the cell surface provides a mechanism for CD44-mediated tumor invasion. Genes Dev 13, 35-48.
Yu, Q., and Stamenkovic, I. (2000). Cell surface-localized matrix metalloproteinase-9 proteolytically activates TGF-beta and promotes tumor invasion and angiogenesis. Genes Dev 14, 163-176.
Yu, Q., Toole, B. P., and Stamenkovic, I. (1997). Induction of apoptosis of metastatic mammary carcinoma cells in vivo by disruption of tumor cell surface CD44 function. J Exp Med 186, 1985-1996.
Yu, W. H., Woessner, J. F., Jr., McNeish, J. D., and Stamenkovic, I. (2002). CD44 anchors the assembly of matrilysin/MMP-7 with heparin-binding epidermal growth factor precursor and ErbB4 and regulates female reproductive organ remodeling. Genes Dev 16, 307-323.
Zawadzki, V., Perschl, A., Rosel, M., Hekele, A., and Zoller, M. (1998). Blockade of metastasis formation by CD44-receptor globulin. Int J Cancer 75, 919-924.
Zeng, Q., and Hong, W. (2008). The emerging role of the hippo pathway in cell contact inhibition, organ size control, and cancer development in mammals. Cancer Cell 13, 188-192.
Zhang, J., Yang, P. L., and Gray, N. S. (2009). Targeting cancer with small molecule kinase inhibitors. Nat Rev Cancer 9, 28-39.
Zhu, B., Suzuki, K., Goldberg, H. A., Riffling, S. R., Denhardt, D. T., McCulloch, C. A., and Sodek, J. (2004). Osteopontin modulates CD44-dependent chemotaxis of peritoneal macrophages through G-protein-coupled receptors: evidence of a role for an intracellular form of osteopontin. J Cell Physiol 198, 155-167.
Zobel, K., Wang, L., Varfolomeev, E., Franklin, M. C., Elliott, L. O., Wallweber, H. J., Okawa, D. C., Flygare, J. A., Vucic, D., Fairbrother, W. J., et al. (2006). Design, synthesis, and biological activity of a potent Smac mimetic that sensitizes cancer cells to apoptosis by antagonizing IAPs. ACS Chem Biol 1, 525-533.
Zou, H. Y., Li, Q., Lee, J. H., Arango, M. E., McDonnell, S. R., Yamazaki, S., Koudriakova, T. B., Alton, G., Cui, J. J., Kung, P. P., et al. (2007). An orally available small-molecule inhibitor of c-Met, PF-2341066, exhibits cytoreductive antitumor efficacy through antiproliferative and antiangiogenic mechanisms. Cancer Res 67, 4408-4417.

1. A method for treating a cancer in a mammal, comprising administering to the mammal in need of such treatment an effective amount for treating the cancer a CD44 fusion protein comprising the constant region of human IgG1 fused to an extracellular domain of CD44, wherein the cancer is a member selected from the group consisting of glioma, colon cancer, breast cancer, prostate cancer, ovarian cancer, lung cancer, renal cell carcinoma, gastric cancer, esophageal cancer, head-neck cancer, pancreatic cancer, liver cancer, and melanoma.

2. (canceled)

3. (canceled)

4. The method according to claim 1, wherein the glioma is a glioblastoma multiforme.

5. The method according to claim 1, wherein the mammal is a human.

6. The method according to claim 1, wherein the extracellular domain of CD44 is a member selected from the group consisting of CD44s, CD44v3-v10, CD44v8-v10, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, CD44sR41A, CD44v3-v10R41A, CD44v8-v10R41A, CD44v4-v10R41A, CD44v5-v10R41A, CD44v6-v10R41A, CD44v7-v10R41A, CD44v9-v10R41A, CD44v10R41A, CD44v9R41A, CD44v8R41A, CD44v7R41A, CD44v6R41A, CD44v5R41A, CD44v4R41A, and CD44v3R41A.

7. The method according to claim 6, wherein the extracellular domain of CD44 is CD44v3-v10.

8. The method according to claim 6, wherein the extracellular domain of CD44 is CD44v8-v10.

9. (canceled)

10. A pharmaceutical composition comprising:
a) a CD44 fusion protein comprising the constant region of human IgG1 fused to an extracellular domain of CD44, wherein the extracellular domain of CD44 is a member selected from the group consisting of CD44v3-v10, CD44v8-v10, CD44s, CD44v4-v10, CD44v5-v10, CD44v6-v10, CD44v7-v10, CD44v9-v10, CD44v10, CD44v9, CD44v8, CD44v7, CD44v6, CD44v5, CD44v4, CD44v3, CD44sR41A, CD44v3-v10R41A, CD44v8-v10R41A, CD44v4-v10R41A, CD44v5-v10R41A, CD44v6-v10R41A, CD44v7-v10R41A, CD44v9-v10R41A, CD44v10R41A, CD44v9R41A, CD44v8R41A, CD44v7R41A, CD44v6R41A, CD44v5R41A, CD44v4R41A, and CD44v3R41A; and
b) a pharmaceutically acceptable carrier or diluent.

11. The pharmaceutical composition of claim 10, wherein the extracellular domain of CD44 is CD44v3-v10.

12. The pharmaceutical composition of claim 10, wherein the extracellular domain of CD44 is CD44v8-v10.

13. (canceled)

14. The pharmaceutical composition of claim 10, wherein the extracellular domain of CD44 is CD44v3-v10R41A.

15. The pharmaceutical composition of claim 10, wherein the extracellular domain of CD44 is CD44v8-v10R41A.

16. The pharmaceutical composition of claim 10, wherein the extracellular domain of CD44 is CD44sR41A.

17. (canceled)

18. (canceled)

19. (canceled)

20. (canceled)

21. (canceled)

22. The method according to claim 1, further comprising administering an additional anti-cancer therapy, wherein the additional anti-cancer therapy is selected from the group consisting of surgery, chemotherapy, radiation therapy, targeted therapy, and immunotherapy.

23-73. (canceled)

74. The pharmaceutical composition of claim 10 further comprising an additional anti-cancer therapy, wherein the additional anti-cancer therapy is selected from the group consisting of surgery, chemotherapy, radiation therapy, targeted therapy, and immunotherapy.

75. The pharmaceutical composition of claim 74, wherein the targeted therapy is against the target selected from the group consisting of EGFR, erbB-2, erbB-3, erbB-4, and c-Met RTK in cancer cells.

76. The method of claim 22, wherein the targeted therapy is against the target selected from the group consisting of EGFR, erbB-2, erbB-3, erbB-4, and c-Met RTK in cancer cells.

77. The method of claim 1, wherein the extracellular domain of CD44 is CD44v3-v10R41A.

78. The method of claim 1, wherein the extracellular domain of CD44 is CD44v8-v10R41A.

79. The method of claim 1, wherein the extracellular domain of CD44 is CD44sR41A.
